(19) United States
(12) Patent Application Publication
(10) Pub. No.: US 2025/0090554 A1
(43) Pub. Date: Mar. 20, 2025

(54) A METHOD OF TREATING DEPRESSION BY IMMUNE MODULATION

(71) Applicants: MYND LIFE SCIENCES INC., Kelowna (CA); The University of Bristish Columbia, Vancouver (CA)

(72) Inventor: Wilfred A. Jefferies, Surrey (CA)

(21) Appl. No.: 18/726,726

(22) PCT Filed: Nov. 4, 2022
(86) PCT No.: PCT/CA2022/051634

§ 371 Date: Jul. 3, 2024

Related U.S. Application Data
(60) Provisional application No. 63/284,787, filed on Dec. 1, 2021.
(60) Provisional application No. 63/275,997, filed on Nov. 5, 2021.

(51) Int. Cl. A61K 31/00; A61K 31/675

(52) U.S. Cl. CPC A61K 31/658; A61K 31/675

(57) ABSTRACT

The present invention relates to methods of immune modulation. In particular, the present invention relates to regulation of neuroinflammation by modulation of ABCF1. Modulation of ABCF1 may be useful in the MDD.

### FIELD OF THE INVENTION

The present invention relates to methods of treating depression by immune modulation. In particular, the present invention relates to treatment of depression by immune modulation through modulation of ABCF1.

### BACKGROUND

Depression affects approximately 10% of humans globally, and the World Health Organization predicts it will become the third most prevalent disease in the world. Evidence indicates that some forms of anxiety and Major Depressive Disorder (MDD) are associated with chronic inflammation and autoimmune diseases including RA, and chronic inflammatory bowel diseases, such as Crohn's Disease (CD). MDD appears to be caused by both genetic and environmental factors, and its diagnosis and management is clinically challenging both because of its unpredictable presentation and response to treatment. Furthermore, MDD is associated with premature mortality from suicide. A traditional hypothesis is that patients living with MDD have a deficiency in brain monoamine neurotransmitters. However, some forms of MDD may be viewed as a psycho-neuroimmunological disorder, which may help to explain why therapies to reduce chronic inflammation also reduce depressive symptoms. Over the last decade, it has become increasingly apparent that several antidepressants possess anti-inflammatory properties. Mechanistically, antidepressants reduce levels of circulating pro-inflammatory cytokines (e.g. IL-1β, TNFα, and IL-6), and reciprocally increase levels of anti-inflammatory cytokines, including IL-10. Consequently, this altered cytokine milieu can modulate serotonergic signaling in neurons and thereby influence emotional and cognitive processing. Overall, these observations suggest that targeting cytokine regulatory pathways can be an effective and novel approach to treat depression.

Escitalopram, an antidepressant of the SSRI (selective serotonin receptor inhibitor) class, has been reported to influence anti-inflammatory pathways in patient populations and it was concluded that ABCF1, an E2 ubiquitin conjugating enzyme, which functions as a strong negative regulator of pro-inflammatory responses is Escitalopram's putative therapeutic target.

### SUMMARY OF THE INVENTION

An object of the present invention is to provide a method of treating depression by immune modulation. In accordance with an aspect of the present invention, there is provided a method of upregulating ABCF1 expression in a patient in need thereof, comprising administering one or more agonists of ABCF1.

### BRIEF DESCRIPTION OF THE FIGURES

These and other features of the invention will become more apparent in the following detailed description in which reference is made to the appended drawings.

FIG. 1 illustrates that Escitalopram induces ABCF1 in a Macrophage cell line: RAW macrophages were plated at 1×105 cells/well and cultured for 2 days. The cells were incubated with 0.3 mM Escitalopram for 1 hour, and then harvested for total RNA, which was extracted for real time RT-PCR specific for ABCF1 and IL-4. CT values were normalized with CT value for the housekeeping gene from the DMSO control. The difference in the expression after drug treatment is consistent with polarization towards an M2-like phenotype (data were consistent in 3 separate experiments).

FIG. 2 illustrates the effect of psylocibin, psylocin and their analogs on ABCF1 transcription in a macrophage cell line. Briefly, the Macrophage cell line RAW264.7 (ATCC) were grown to 80% confluency in growth media (DMEM+10% FBS+glutamine). Dilutions of psylocibin, psylocin and their analogs were made at desired final concentrations for a Dose response experiment. The concentrations' used for this experiment are: 10 nM, 100 nM, 500 nM for Psilocin, Psylocibin, 4-Acetoxy-N, N-dimthyltryptamine, O-Acetyl Psilocin Fumerate, and 4-acetoxyindole. The cells were incubated for 2 hours and then harvested for total RNA, which was extracted for real time RT-PCR specific for ABCF1. Untreated cells were used as negative control and Escitalopram at 0.3 mM was used as a positive control to activate ABCF1 expression for all the experiments. ES=escitalopram; PSYB=Psylocibin; PSIC=Psilocin; DMT=4-Acetoxy-N, N-dimthyltryptamine; APF=O-Acetyl Psilocin Fumerate, and AOI=4-acetoxyindole.

FIG. 3 illustrates the effect of Escitalopram (positive control) and Psilocybin-analogs treatment on ABCF1-reporter macrophage cell line (RAW 264) (in vitro). Macrophage cell line RAW264.7 (ATCC) was grown to 80% confluency in growth media (DMEM+10% FBS+glutamine). Dilutions of the drugs were made at desired final concentrations for a Dose-response experiment. The concentrations used for this experiment were: 10 nM, 100 nM, 500 nM for psilocybin analogs. In accordance with preliminary data, the concentration of Escitalopram was set at 0.3 mM to induce ABCF1 expression. The cells were incubated for 2 hours and then harvested for total RNA, which was extracted for real time RT-PCR specific for ABCF1. Untreated cells were used as negative control and Escitalopram at 0.3 mM was used as a positive control to activate ABCF1 expression for all the experiments. DMT=4-Acetoxy-N, N-dimthyltryptamine; APF=O-Acetyl Psilocin Fumerate, and AOI=4-acetoxyindole; AIMT=4-Acetoxy-N-isopropyl-N-methyltryptamine; AEMT—4-Acetoxy-N-ethyl-N-methyltryptamine; AODET=-Acetoxy-N,N-diethyltryptamine, also known as 4-AcO-DET; AODET-F=4-AcO-DET Fumarate and AOMET-F=4-Acetoxy-N-ethyl-N-methyltryptamine Fumarate.

FIG. 4 illustrates the effect of Escitalopram (positive control) and Psilocybin-analogs treatment on ABCF1-reporter macrophage cell line (RAW 264) (in vitro). Macrophage cell line RAW264.7 (ATCC) was grown to 80% confluency in growth media (DMEM+10% FBS+glutamine). Dilutions of the drugs were made at desired final concentrations for a Dose-response experiment. The concentrations used for this experiment were: 10 nM, 100 nM, 500 nM for psilocybin analogs. In accordance with preliminary data, the concentration of Escitalopram was set at 0.3 mM to induce ABCF1 expression. The cells were incubated for 24 hours and then harvested for total RNA, which was extracted for real time RT-PCR specific for ABCF1. Untreated cells were used as negative control and Escitalopram at 0.3 mM was used as a positive control to activate ABCF1 expression for all the experiments. DMT=4-Acetoxy-N, N-dimthyltryptamine; APF=O-Acetyl Psilocin Fumerate, and AOI=4-acetoxyindole.

FIG. 5 illustrates ABCF1 expression in RAW cells treated with drugs. ABCF1. ABCF1 is induced at 2 hours with different psychedelic drugs and returns to base line at 24 hours in RAW cells. ABCF1 is reinduced post a second 2 hour treatment (26 hr time point) Indicating potential for Micro-dosing

FIG. 6 illustrates ABCF1 expression in RAW cells treated with cannabigerol. ABCF1 expression levels increased by 8 folds in murine macrophage cell line, RAW264.7 cells, post 2 hr treatment with 100 nM of Cannabigerol.

FIG. 7 illustrates ABCF1 expression in bone marrow cells post 30 minutes treatment in vivo. ABCF1 expression levels increased by 6 folds bone marrow after 30 mins treatment with Escitalopram and 2 folds after treatment with psilocybin.

FIG. 8 illustrates changes in ABCF1 gene expression in cell cultures and in mouse models. (A) Escitalopram (0.6 nM) and Psilocybin (500 nM) induces ABCF1 expression when added to RAW macrophages cell cultures for 2 hrs; (B) Escitalopram (50 mg/kg) and Psilocybin (25 mg/kg) induces ABCF1 expression in the bone marrow when given to mice for 30 minutes; (C) Escitalopram (50 mg/kg) and psilocybin (25 mg/kg) induce ABCF1 expression in the whole brain when given to mice for 30 minutes. P-values and fold changes were established in relationship to untreated control group.

FIG. 10 illustrates the effect of psilocybin and escitalopram on ABCF1 brain gene expression (bottom panel). Top panel is a legend of the cell types.

FIG. 11 illustrates the changes in ABCF1 expression in bone marrow, brain and PBMCs 30 minutes post treatment with Escitalopram or Psilocybin.

FIG. 12 illustrates the changes in ABCF1 expression in bone marrow and brain 30 minutes post Drug treatment after 30 minutes LPS stimulation (PBS control, Escitalopram 50 mg/kg, O-Acetyl Psilocin Fumarate (APF) 25 mg/kg, 4-AcetoxyIndole (AOI) 2.5 mg/kg, 4-AcO-DET (AODET) 25 mg/kg).

### DETAILED DESCRIPTION

The present invention is based on the discovery that some forms of anxiety and Major Depressive Disorder (MDD) are associated with chronic inflammation and certain antidepressants possess anti-inflammatory properties. Accordingly, in certain embodiments, the present invention provides a method of inhibiting inflammation, including but not limited to neuroinflammation to treat neuropsychiatric disorders, including but not limited to Major Depressive Disorder (MDD), postpartum depression, schizophrenia, anxiety, bipolar disorder, obsessive-compulsive disorder (OCD), posttraumatic stress disorder (PTSD), and autism spectrum disorder.

Accordingly, in certain embodiments, the present invention provides methods of treating Major Depressive Disorder (MDD) or alleviating one or more symptoms of MDD. Symptoms of MDD include but are not limited to trouble concentrating, remembering details, and making decisions; fatigue; feelings of guilt, worthlessness, and helplessness; pessimism and hopelessness; insomnia, early-morning wakefulness, or sleeping too much; irritability; restlessness; loss of interest in things once pleasurable, including sex; overeating, or appetite loss; aches, pains, headaches, or cramps that won't go away; digestive problems that don't get better, even with treatment; persistent sad, anxious, or “empty” feelings; and suicidal thoughts or attempts.

ABCF1, an E2 ubiquitin conjugating enzyme, is a strong negative regulator of pro-inflammatory responses. Accordingly, neuroinflammation may be inhibited by upregulating the expression and/or activity of ABCF1. In certain embodiments, the present invention provides methods of inhibiting neuroinflammation to treat neuropsychiatric disorders by upregulating the expression and/or activity of ABCF1. In specific embodiments, the present invention provides methods of treating Major Depressive Disorder (MDD) or alleviating one or more symptoms of MDD by upregulating the expression and/or activity of ABCF1.

It is known in the art that MDD is common in patients with autoimmune diseases, such as Rheumatoid Arthritis, inflammatory bowel disease, multiple sclerosis and pancreatitis.

It is also known that inhibition of inflammation and/or an immune response may also be useful in the prevention and/or treatment of such autoimmune diseases. Accordingly, methods which treat autoimmune diseases by inhibiting inflammation and/or an immune response may also be useful in the treatment of MDD in autoimmune patients having comorbid MDD.

Accordingly, in certain embodiments, the present invention provides methods of preventing and/or treating autoimmune and comorbid neuropsychiatric disorders. In certain embodiments, the present invention provides treatments for inflammatory autoimmune disease and neuropsychiatric disorders associated with neuroinflammation by immune modulation. In certain embodiments, the present invention provides combined treatments for inflammatory autoimmune disease and neuropsychiatric disorders by upregulating ABCF1 expression and/or activity. In specific embodiments, the present invention provides treatment for Rheumatoid Arthritis comorbid Major Depressive Disorder by upregulating ABCF1 expression and/or activity. Treatment may include one or more methods to enhance expression and/or activity of ABCF1 alone or in combination with other therapeutics.

Non-limiting examples of methods to enhance expression and/or activity of ABCF1, include administration of the ABCF1, or active fragments thereof, administration of a nucleic acid or vector which encodes the ABCF1 or administration of one or more molecules which enhance expression of ABCF1 (one or more agonists).

The ABCF1 protein and nucleic acid sequences (genomic and cDNA) are known in the art. See for example GenBank Accession numbers AQY76226.1, AQY76225.1, KY500135.1 and KY500134.1. In certain embodiments, the ABCF1 comprises the sequence set forth below:

MPKAPKQQPPEPEWIGDGESTSPSDKVVKKGKKDKKIKKTFFEELAVED
KAGEEEKVLKEKEQQQQQQQQQQKKKRDTRKGRRKKDVDDDGEEKELME
RLKKLSVPTSDEEDEVPAPKPRGGKKTKGGNVFAALIQDQSEEEEEEEK
HPPKPAKPEKNRINKAVSEEQQPALKGKKGKEEKSKGKAKPQNKFAALD
NEEEDKEEEIIKEKEPPKQGKEKAKKAEQGSEEEGEGEEEEEEGGESKA
DDPYAHLSKKEKKKLKKQMEYERQVASLKAANAAENDFSVSQAEMSSRQ
AMENASDIKLEKFSISAHGKELFVNADLYIVAGRRYGLVGPNGKGKTTL
LKHIANRALSIPPNIDVLLCEQEVVADETPAVQAVLRADTKRLKLLEEE
RRLQGQLEQGDDTAAERLEKVYEELRATGAAAAEAKARRILAGLGFDPE
MQNRPTQKFSGGWRMRVSLARALFMEPTLLMLDEPTNHLDLNAVIWLNN
YLQGWRKTLLIVSHDQGFLDDVCTDIIHLDAQRLHYYRGNYMTFKKMYQ
QKQKELLKQYEKQEKKLKELKAGGKSTKQAEKQTKEALTRKQQKCRRKN
QDEESQEAPELLKRPKEYTVRFTFPDPPPLSPPVLGLHGVTFGYQGQKP
LFKNLDFGIDMDSRICIVGPNGVGKSTLLLLLTGKLTPTHGEMRKNHRL
KIGFFNQQYAEQLRMEETPTEYLQRGFNLPYQDARKCLGRFGLESHAHT
IQICKLSGGQKARVVFAELACREPDVLILDEPTNNLDIESIDALGEAIN
EYKGAVIVVSHDARLITETNCQLWVVEEQSVSQIDGDFEDYKREVLEAL
GEVMVSRPRE.

Appropriate vectors are known in the art and include but are not limited to adenoviral vectors.

Molecules known to enhance the ABCF1 pathway include but are not limited to Escitalopram, an antidepressant of the SSRI. A number of psilocybins have immune modulatory activities and may enhance the ABCF1 pathway. Accordingly, in certain embodiments, the methods comprise administration of psilocybins and/or antidepressant drugs to modulate the ABCF1 pathway.

In certain embodiments, the molecule is escitalopram or molecules structurally similar to escitalopram.

In certain embodiments, the molecule is selected from any one of the following:

Escitalopram oxalate
Escitalopram hydrobromide
Escitalopram
Escitalopram(1+)
Citalopram
1-[4-Bromo-2-(hydroxymethyl)phenyl]-4-
(dimethylamino)-1-(4-fluorophenyl)butan-1-ol
(S)-Citalopram N-Oxide
Desmethyl Citalopram Hydrobromide
2,3-Bis[(4-methylbenzoyl)oxy]butanedioic acid;
4-[4-(dimethylamino)-1-(4-fluorophenyl)-1-
hydroxybutyl]-3-(hydroxymethyl)benzonitrile
4-[4-(Dimethylamino)-1-(4-fluorophenyl)-1-hydroxybutyl]-3-
(hydroxymethyl)benzonitrile; methane
3-[(1S)-1-(4-Fluorophenyl)-5-isocyano-
3H-2-benzofuran-1-yl]-N-methyl-N-
(trideuteriomethyl)propan-1-amine; oxalic acid
1-(4-Fluorophenyl)-1-(3-dimethylaminopropyl)-
5-chlorophthalane
4-[(Z)-4-(Dimethylamino)-1-(4-fluorophenyl)but-
1-enyl]-3-(hydroxymethyl)benzonitrile
1,3-Dihydro-1-oxoisobenzofuran-5-carboxamide
1-(3-Dimethylaminopropyl)-1-(4-fluorophenyl)-
1,3-dihydroisobenzofuran-5-carboxamide
Citalopram Oxalate
desmethylcitalopram (DCT)
didesmethylcitalopram (DDCT)
(RS)-1-[3-(Dimethylamino)propyl]-1-(4-fluorophenyl)-
1,3-dihydroisobenzofuran-5-carbonitrile
citalopram propionaldehyde
citalopram propionic acid
(RS)-1-[3-(methylethylamino)propyl]-1-(4-
fluorophenyl)-1,3-dihydroisobenzofuran-5-
carbonitrile
(RS)-1-[3-(Dieethylamino)propyl]-1-(4-fluorophenyl)-
1,3-dihydroisobenzofuran-5-carbonitrile

In certain embodiments, the molecule is a derivative of escitalopram. In certain embodiments, the middle carbon atom middle carbon atom of the propyl linkage from the N atom to the chiral centre comprises a methyl or ethyl substitution. In certain embodiments, the 4-fluorophenyl group in the 2 and/or 6 positions (meta to the F) is substituted. For example, the 2,4-difluorophenyl substructure is known in 1-(2,4-difluorophenyl) methanamine and the 2,4,6-trifluorophenyl substructure is known in 2,4,6-Trifluorophenyl) methanamine-both of which are available as reagents. In certain embodiments, the molecule is a deuterated escitalopram.

In certain embodiments, the molecule is a psilocybin or derivative thereof. In specific embodiments, the molecule is any one of the following:

Psilocybine
Psilocin
4-Hydroxytryptamine
O-Methylpsilocin
4-Hydroxy-N,N diethyltryptamine
3-(2-Pyrrolidin-1-yl-ethyl)-1H-indol-4-ol
Indol-6-OL, 3-(2-(dimethylamino)ethyl)-
4-Hydroxy-N-methyltryptamine 4-phosphate
4-Hydoxytryptamine 4-phosphate
3-(2-Dimethylamino-propyl)-1H-indol-4-ol
4-Hydroxy-N,N-diisopropyltryptamine
4-Hydroxy-N-methyl-N-ethyltryptamine
4-Hydroxy-N-methyl-N-isopropyltryptamine
[3-[2-(Dimethylamino)ethyl]-1-methylindol-
4-yl] dihydrogen phosphate
Ethocybin
3-(3-Dimethylamino-propyl)-1H-indol-4-ol
3-Dimethylaminomethyl-1H-indol-4-ol
[1-Butyl-3-[2-(dimethylamino)ethyl]indol-
4-yl] dihydrogen phosphate
2-(4-Hydroxy-1H-indol-3-yl)ethanaminium
3-[2-(Dimethylazaniumyl)ethyl]-1H-indol-4-yl phosphate
3-[2-(4-Methyl-piperazin-1-yl)-ethyl]-1H-indol-4-ol
Meprocin
3-[2-(Methylamino)ethyl]-1H-indol-4-ol
3-(2-Dimethylamino-ethyl)-6-fluoro-1H-indol-4-ol
3-(1-Methyl-pyrrolidin-3-yl)-1H-indol-4-ol
Norbaeocystin
Baeocystin
[3-[2-(Trimethylazaniumyl)ethyl]-1 H-indol-4-yl] phosphate
N,N-Diallyl-4-hydroxytryptamine
Aeruginascin
n,n-Diisopropyl-4-hydroxytryptamine hydrochloride
Dipropyl-4-hydroxytryptamine
3-[(2R)-2-Aminopropyl]-1H-indol-4-ol
3-[(2S)-2-Aminopropyl]-1H-indol-4-ol
(R)-3-((1-Methylpyrrolidin-2-YL)methyl)-1H-indol-4-OL
3-Methyl-1H-indol-4-ol
O-Benzyl Psilocybin
3-(2-Methylbut-3-en-2-yl)-1H-indol-4-ol
1H-Indol-4-olate
2-(4-Dibenzylphosphoryloxy-1H-indol-3-yl)-
N,N-dimethylethanamine
4-Dibenzylphosphoryloxy-3-(2-piperidin-
1-ylethyl)-1H-indole
1H-Indol-4-yl phosphate
Tris [[3-[2-(dimethylamino)ethyl]-1H-indol-
4-yl]oxy]-hydroxyphosphanium
3-(2-Azidoethyl)-1H-indol-4-ol
3-(4-Azidooxy-1H-indol-3-yl)-N,N-
dimethylpropan-1-amine
4-[Azido(methyl)phosphoryl]oxy-1H-indole
[3-[2-(Dimethylamino)ethyl]-1H-indol-4-yl] phosphate
[3-[2-[Benzyl(dimethyl)azaniumyl]ethyl]-
1H-indol-4-yl] phosphate
4-Azidooxy-3-methyl-1H-indole
1H-Indol-4-yl sulfate
2-[1H-Indol-3-yloxy(methoxy)phosphoryl]oxyethyl-
trimethylazanium
[3-[2-(Dimethylamino)ethyl]-1H-indol-4-yl]oxy-
methylphosphinic acid
2-(4-Dihydroxyphosphanyloxy-1H-indol-3-
yl)ethyl-trimethylazanium
[3-(3-Methylbutyl)-1H-indol-4-yl] phosphono
hydrogen phosphate
2-(4-Dimethylphosphoryloxy-1H-indol-3-yl)-
N,N-dimethylethanamine
2-[4-[Hydroxy(phenylmethoxy)phosphoryl]oxy-
1H-indol-3-yllethyl-dimethylazanium
3-[2-[Amino(methyl)amino]ethyl]-1H-indol-4-ol
Benzyl-[2-[4-[hydroxy(methyl)phosphoryl]oxy-
1H-indol-3-yl]ethyl]-dimethylazanium
3-[2-[lodo(methyl)amino]ethyl]-1H-indol-4-ol
Potassium; 3-(2-aminoethyl)-1H-indol-4-olate
N-(1H-Indol-4-yloxy)-2-methylpropan-1-amine;
methanol
O-[3-(2-Aminoethyl)-1H-indol-4-yl]hydroxylamine;
fluoromethane
N,N-Dimethyl-2-(4-sulfanyloxy-1H-indol-3-yl)ethanamine;
ethane
Ethane; methane; O-[3-[2-(methylamino)ethyl]-
1H-indol-4-yl]hydroxylamine
3-Ethyl-4-sulfanyloxy-1H-indole; methane
(3-Heptan-3-yl-1H-indol-4-yl) hypofluorite; methane
Ethane; N-methyl-2-(4-sulfanyloxy-1H-indol-3-yl)ethanamine
Fluoromethane; (3-heptan-3-yl-1H-indol-4-yl) hypofluorite
Fluoromethane; O-[3-[2-(methylamino)ethyl]-
1H-indol-4-yl]hydroxylamine
Methane; (3-pentan-3-yl-1H-indol-4-yl) hypofluorite; propane
Fluoromethane; octane; O-(3-propyl-1H-
indol-4-yl)hydroxylamine
[3-(3-Ethylheptyl)-1H-indol-4-yl] hypofluorite; methane
3-(2-Aminoethyl)-1H-indol-4-ol; ethane
3-[2-(Dimethylamino)ethyl]-1H-indol-4-ol; ethane
1H-Indol-4-yloxyboronic acid
4-Methylsulfanyloxy-1H-indole
2-(5-Phosphorosooxy-1H-indol-3-yl)ethanamine
[3-[2-(Dimethylamino)ethyl]-1H-indol-4-yl]
phosphono hydrogen phosphate
Benzyl-dimethyl-[2-(4-phosphonooxy-1H-
indol-3-yl)ethyl]azanium
[3-[2-(Dimethylazaniumyl)ethyl]-1H-indol-
4-yl] hydrogen phosphate
[3-[2-(Dimethylamino)ethyl]-1H-indol-4-yl]
dihydrogen phosphate; hydrate
Dibenzyl [3-[2-(dimethylamino)ethyl]-1 H-indol-4-yl] phosphate
3-[2-[Ethyl(propyl)amino]ethyl]-1H-indol-4-ol
[3-(1-Aminoethyl)-1H-indol-4-yl] dihydrogen phosphate
[3-[2-(Dimethylamino)ethyl]-1H-indol-4-yl] hydrogen phosphate
[1-(Aminomethyl)-3-[2-(methylamino)ethyl]indol-4-yl] hypoiodite
2-[4-(Methoxymethylphosphanyloxy)-1 H-indol-
3-yl]-N,N-dimethylethanamine
(2S)-2-[[3-[2-(Dimethylamino)ethyl]-1H-indol-4-yl]oxy-
(methoxymethyl)phosphoryl]amino]-2,3-
dimethylbutanoic acid
Dibromo 1H-indol-4-yl phosphate
3-[2-(Diethylamino)ethyl]-1H-indol-6-ol
3-[2-(Ethylamino)ethyl]-1H-indol-4-ol
1H-Indol-5-yl dihydrogen phosphate
3-[2-(Methylamino)ethyl]-1-propan-2-ylindol-4-ol
1H-Indol-4-yl hypofluorite
[4-(1-Ethyl-3,6-dihydro-2H-pyridin-5-yl)-
1H-indol-3-yl] dihydrogen phosphate
3-[2-(Dimethylamino)ethyl]-5-fluoro-1H-indol-4-ol
3-[2-(Dimethylamino)ethyl]-1H-indol-4-ol; [3-[2-
(dimethylamino)ethyl]-1H-indol-4-yl] dihydrogen
phosphate
4-Phosphorosooxy-1H-indole
1H-Indol-4-yl dihydrogen phosphate
O-[3-[2-(Methylamino)ethyl]-1H-indol-6-yl]hydroxylamine
3-(2-Methyl-1,2,3,6-tetrahydropyridin-4-yl)-1H-indol-4-ol
CID 67465946
1H-Indol-4-yloxy(oxo)borane
[3-[2-(Trimethylazaniumyl)ethyl]-1H-indol-4-
yl] hydrogen phosphate
[3-[2-(Dimethylamino)ethyl]-7-fluoro-1-methylindol-
4-yl] dihydrogen phosphate
[3-[2-(Dimethylamino)ethyl]-7-fluoro-1-
propylindol-4-yl] dihydrogen phosphate
1-Ethyl-3-[2-(methylamino)ethyl]indol-4-ol; tungsten
CID 58110672
2-(4-Phosphorosooxy-1H-indol-3-yl)ethanamine
N,N-Dimethyl-2-(4-phosphorosooxy-1H-indol-3-yl)ethanamine
4-Hydroperoxy-1H-indole
1H-Indol-4-yl hypochlorite
3-Butyl-1H-indol-4-ol
1H-Indol-4-yl nitrate
O-(1H-Indol-4-yl)hydroxylamine
1H-Indol-4-yl hypobromite
1H-Indol-4-yl hypoiodite
3-[(1-Methylpyrrolidin-2-yl)methyl]-1H-indol-4-ol
4-Methylperoxy-1H-indole
N,N-Diethyl-2-(4-methoxy-1H-indol-3-yl)ethanamine
3-[[2-(Dimethylamino)phenyl]methyl]-1H-indol-4-ol
3-(2-Piperidinoethyl)-1H-indole-4-ol
3-[2-(Dibutylamino)ethyl]-1H-indol-4-ol
4-HO-Dsbt
3-(Aminomethyl)-1H-indol-4-ol
1H-Indol-4-ol, 3-ethyl-
4-Hydroxy-3-(1-methyl-1,2,3,6-tetrahydropyridin-4-yl)-1H-indole
4-Hydroxy-3-(1-benzyl-1,2,3,6-tetrahydropyridin-4-yl)-1H-indole
4-Hydroxy-3-(1,2,3,6-tetrahydropyridin-4-yl)-1H-indole
1-(4-Hydroxy-1H-indol-3-yl)ethyl-trimethylazanium
2-(4-Hydroxy-1H-indol-3-yl)ethyl-trimethylazanium
2-(4-Hydroxy-1H-indol-3-yl)ethyl-trimethylazanium;
methyl sulfate
(4-Hydroxy-1H-indol-3-yl)methyl-trimethylazanium
(4-Hydroxy-1H-indol-3-yl)methyl-trimethylazanium;
methyl sulfate
Benzyl [3-[2-[benzyl(dimethyl)azaniumyl]ethyl]-
1H-indol-4-yl] phosphate
2-[Hydroxy(1H-indol-3-yloxy)phosphoryl]oxyethyl-
trimethylazanium
1H-Indol-3-yl (2-(trimethylammonio)ethyl) phosphate
3-[2-[Methyl((111C)methyl)amino]ethyl]-1H-indol-4-ol
3-[[(2S)-1-Methyl-2-pyrrolidinyl]methyl]-1H-indole-4-ol
Benzyl-[2-[4-[hydroxy(phenylmethoxy)phosphoryl]oxy-
1H-indol-3-yllethyl]-dimethylazanium
1H-Indol-4-ol, 3-(2-aminopropyl)-
3-[2-[Bis(2-methylpropyl)amino]ethyl]-1H-indol-4-ol
3-[2-[Butyl(methyl)amino]ethyl]-1H-indol-4-ol
3-[2-[Methyl(2-methylpropyl)amino]ethyl]-1H-indol-4-ol
3-[2-[Butan-2-yl(methyl)amino]ethyl]-1H-indol-4-ol
3-[2-[Tert-butyl(methyl)amino]ethyl]-1H-indol-4-ol
3-[2-[Cyclopentyl(methyl)amino]ethyl]-1H-indol-4-ol
3-[2-(2,6-Dimethylpiperidin-1-yl)ethyl]-1H-indol-4-ol
3-[2-(Dimethylamino)ethyl]-1H-indol-4-ol; phosphoric acid
2-(4-Hydroxy-1H-indol-3-yl)ethyl-dimethylazanium
4-Hydroxy-N-isopropyl-N-methyltryptamine-d4
1-Ethyl-3-[2-(methylamino)ethyl]indol-4-ol
3-[2-(N,2-Dimethylanilino)ethyl]-1H-indol-4-ol
Benzyl [3-[1,1,2,2-tetradeuterio-2-(dimethylamino)ethyl]-
1H-indol-4-yl] hydrogen phosphate
N,N-Dimethyl-2-(4-trimethylsilyloxy-1H-indol-3-yl)ethanamine
3-[1,1,2,2-Tetradeuterio-2-[ethyl(methyl)amino]ethyl]-
1H-indol-4-ol
3-[1,1,2,2-Tetradeuterio-2-(diethylamino)ethyl]-1H-indol-4-ol
3-[1,1,2,2-Tetradeuterio-2-(dimethylamino)ethyl]-1H-indol-4-ol
[3-[1,1,2,2-Tetradeuterio-2-(dimethylamino)ethyl]-
1H-indol-4-yl] dihydrogen phosphate
1H-Indol-4-ol, 3-(3-aminopropyl)-
3-[2-(Methylamino)propan-2-yl]-1H-indol-4-ol
3-Methyl-5-[2-(methylamino)ethyl]-1H-indol-4-ol
5-(3-Aminopropyl)-3-methyl-1H-indol-4-ol
3-(2-Aminoethyl)-1-methylindol-4-ol
1-Methyl-3-[2-(methylamino)ethyl]indol-4-ol
3-(3-Aminopropyl)-1-methylindol-4-ol
3-(1-Aminopropan-2-yl)-1-methylindol-4-ol
3-(2-Aminopropyl)-1-methylindol-4-ol
3-(Methylaminomethyl)-1H-indol-4-ol
3-[2-(Methylamino)propyl]-1H-indol-4-ol
3-(2-Aminoethyl)-1-propan-2-ylindol-4-ol
1-Methyl-3-[2-(methylamino)propyl]indol-4-ol
3-(3-Aminopropyl)-1-propan-2-ylindol-4-ol
3-(2-Aminopropyl)-1-propan-2-ylindol-4-ol
3-(4-Piperidinyl)-1H-Indol-4-ol
1-Methyl-3-(1,2,3,6-tetrahydropyridin-4-yl)indol-4-ol
1-(3-Aminopropyl)-3-methylindol-4-ol
3-(2-Aminoethyl)-5-ethyl-1H-indol-4-ol
3-(Fluoromethyl)-1H-indol-4-ol
3-(1-Fluoroethyl)-1H-indol-4-ol
3-(2-Fluoropropan-2-yl)-1H-indol-4-ol
3-(Pyrrolidin-1-ylmethyl)-1H-indol-4-ol
3-(Piperidin-1-ylmethyl)-1H-indol-4-ol
3-(2-Aminoethyl)-1-ethylindol-4-ol
3-(2-Aminoethyl)-1-propylindol-4-ol
3-(2-Aminoethyl)-1-prop-2-enylindol-4-ol
[3-[2-(Dimethylamino)ethyl]-1-trimethylsilylindol-
4-yl] bis(trimethylsilyl) phosphate
3-[2-[Hydroxy(methyl)amino]propan-2-yl]-1H-indol-4-ol
3-[(3R)-1-Methyl-3-pyrrolidinyl]methyl]-1H-indole-4-ol
3-[(3S)-1-Methyl-3-pyrrolidinyl]methyl]-1H-indole-4-ol
3-Propan-2-yl-1H-indol-4-ol
3-[2-[Bis(trideuteriomethyl)amino]-1,1,2,2-
tetradeuterioethyl]-1H-indol-4-ol
3-(1-Aminopropan-2-yl)-1-ethylindol-4-ol
3-(2-Aminopropyl)-1-ethylindol-4-ol
3-(3-Aminopropyl)-1-ethylindol-4-ol
3-[(Cyclobutylamino)methyl]-1H-indol-4-ol
3-[(Cyclopentylamino)methyl]-1H-indol-4-ol
3-(Azepan-1-ylmethyl)-1H-indol-4-ol
3-[(4-Methylpiperazin-1-yl)methyl]-1H-indol-4-ol
3-(Ethylaminomethyl)-1H-indol-4-ol
3-[(Propan-2-ylamino)methyl]-1H-indol-4-ol
3-(Ethylaminomethyl)-1-methylindol-4-ol
1-Methyl-3-[(propan-2-ylamino)methyl]indol-4-ol
1-Ethyl-3-(methylaminomethyl)indol-4-ol
1-Ethyl-3-(ethylaminomethyl)indol-4-ol
1-Ethyl-3-[(propan-2-ylamino)methyl]indol-4-ol
3-(Ethylaminomethyl)-1-propan-2-ylindol-4-ol
1-Propan-2-yl-3-[(propan-2-ylamino)methyl]indol-4-ol
3-(2-Hydroxypropan-2-yl)-1H-indol-4-ol
3-(2-Hydroxy-2-methylpropyl)-1H-indol-4-ol
3-(Aminooxymethyl)-1H-indol-4-ol
1H-Indol-4-ol, 3-(1-aminoethyl)-
3-[1-(Methylamino)ethyl]-1H-indol-4-ol
1-Methyl-3-[1-(methylamino)ethyl]indol-4-ol
3-[3-(Methylamino)propyl]-1H-indol-4-ol
1-Methyl-3-[3-(methylamino)propyl]indol-4-ol
1-Ethyl-3-[1-(methylamino)ethyl]indol-4-ol
3-[1-(Methylamino)ethyl]-1-propan-2-ylindol-4-ol
1-Ethyl-3-[2-(methylamino)propyl]indol-4-ol
3-[2-(Methylamino)propyl]-1-propan-2-ylindol-4-ol
1-Ethyl-3-[3-(methylamino)propyl]indol-4-ol
3-[3-(Methylamino)propyl]-1-propan-2-ylindol-4-ol
5-(4-Aminobutyl)-3-methyl-1H-indol-4-ol
1H-Indol-4-ol, 3-propyl-
3-[2-(Dimethylamino)-1-hydroxyethyl]-1H-indol-4-ol
3-[(3S)-1-Methylpyrrolidin-3-yl]-1H-indol-4-ol
3-[(3R)-1-Methylpyrrolidin-3-yl]-1H-indol-4-ol
3-[1-(Dimethylamino)propan-2-yl]-1H-indol-4-ol
3-(2-Aminobutyl)-1H-indol-4-ol
3-(1-Amino-3-methylbutyl)-1H-indol-4-ol
3-[(1S)-1-Amino-3-methylbutyl]-1H-indol-4-ol
3-[(1R)-1-Amino-3-methylbutyl]-1H-indol-4-ol
3-[1-[Hydroxy(methyl)amino]ethyl]-1H-indol-4-ol
3-(N-Hydroxy-C-methylcarbonimidoyl)-1H-indol-4-ol
[3-[2-(Dimethylazaniumyl)ethyl]-1 H-indol-4-
yl] hydrogen phosphate; methanol
[3-(3-Ethylheptyl)-1 H-indol-4-yl] hypofluorite
O-(3-Propyl-1H-indol-4-yl)hydroxylamine
(3-Pentan-3-yl-1H-indol-4-yl) hypofluorite
O-[3-[2-(Methylamino)ethyl]-1H-indol-4-yl]hydroxylamine
(3-Heptan-3-yl-1H-indol-4-yl) hypofluorite
N-Methyl-2-(4-sulfanyloxy-1H-indol-3-yl)ethanamine
3-Ethyl-4-sulfanyloxy-1H-indole
N,N-Dimethyl-2-(4-sulfanyloxy-1H-indol-3-yl)ethanamine
O-[3-(2-Aminoethyl)-1H-indol-4-yl]hydroxylamine
N-(1H-Indol-4-yloxy)-2-methylpropan-1-amine
1H-Indol-4-yl hydrogen sulfate

In certain embodiments, the molecule is a psilocybin and psilocybin-like compounds. Exemplary compounds include but are not limited to Psilocybin ([3-(2-Dimethylaminoethyl)-1H-indol-4-yl] dihydrogen phosphate), Psilocybin (zwitterion form), Psilocin (4-hydroxy-N,N-dimethyltryptamine), Serotonin (5-Hydroxytryptamine), DMT (N,N-Dimethyltryptamine), Lysergic acid diethylamide (LSD, (6aR,9R)—N,N-diethyl-7-methyl-4,6,6a,7,8,9-hexahydroindolo [4,3-fg]quinoline-9-carboxamide, psilocin iminoquinone, psilocin o-quinone, Trimethylglycine (TMG), Phenyl hydrogen sulfate and indoxyl sulfate.

In certain embodiments, the molecule is a phenylethylamine. Exemplary phenulethylamines include but are not limited to Mescaline (3,4,5-Trimethoxyphenethylamine), Noradrenaline (also called Norepinephrine), DOM, DOI, DOB, Cathinone (] benzoylethanamine, or β-keto-amphetamine) and Amphetamine (alpha-methylphenethylamine.

In certain embodiments, the molecule is any one of the following listed in the table below:

| Common Name | Formal Name | Natural |
|---|---|---|
| Psilocybin | [3-(2-Dimethylaminoethyl)-1H-indol- | YES |
| | 4-yl] dihydrogen phosphate, | |
| | or | |
| | O-phosphoryl-4-hydroxy-N,N- | |
| | dimethyl-tryptamine | |
| Major Metabolites | | |
| Psilocin | 4-hydroxy-N,N-dimethyltryptamine | YES |
| Psilocin-O-glucuronide | [3-(2-Dimethylaminoethyl)-1H- | YES |
| | indol-4-yl] glucuronide | |
| Psilocin iminoquinone | Psilocin iminoquinone | YES |
| Psilocin o-quinone | Psilocin o-quinone | YES |
| Indoylalkylamines | | |
| Serotonin | 5-Hydroxytryptamine | YES |
| Mescaline | 3,4,5-Trimethoxyphenethylamine | YES |
| DMT | N,N-Dimethyltryptamine | YES |
| 5-methoxy-DMT | 5-methoxy-N,N-Dimethyltryptamine | YES |
| Lysergic acid | (6aR,9R)-N,N-diethyl- | |
| diethylamide (LSD) | 7-methyl-4,6,6a,7,8,9- | |
| | hexahydroindolo[4,3- | |
| | fg]quinoline-9-carboxamide | |
| Phenylethylamines | | |
| Noradrenaline | 2,5-Dimethoxy-4-methylamphetamine | YES |
| DOM | 2,5-Dimethoxy-4-methylamphetamine | |
| DOI | 2,5-Dimethoxy-4-iodoamphetamine | |
| DOB | 2,5-Dimethoxy-4-bromoamphetamine | |
| DOC | 2,5-Dimethoxy-4-chloroamphetamine | |
| DOF | 2,5-Dimethoxy-4-fluoroamphetamine | |
| Cathinone | benzoylethanamine, or | YES |
| | β-keto-amphetamine | |
| Predicted Compounds | | |
| Other sugar analogs | Various, replacing the glucuronide | |
| of psilocin-O- | with an alternate sugar | |
| glucuronide | substructure. | |
| All analogs where the | —NH₂ is replaced by NR₂, where | |
| H′s of the terminal | R is a member of the set of | |
| primary amine —H₂ are | small alkyls, methyl, ethyl, | |
| substituted by two | n-propyl, and so forth. | |
| identical groups | | |
| All analogs where the | —N(CH₃)₂ is by NR₂, where R | |
| methyl groups of the | is H or a member of the set of | |
| terminal tertiary amine | small alkyls, ethyl, | |
| —N(CH₃)₂ are | n-propyl, and so forth. | |
| substituted by two | | |
| identical groups | | |
| All analogs where | —NR₂ is replaced by NRR′, where | |
| the substructures | R and R′ are dissimilar | |
| attached to the terminal | members of the set containing H | |
| amine nitrogen | and the small alkyls, ethyl, n- | |
| are dissimilar | propyl, and so forth. | |
| All analogs of psilocybin | —NR₂H+ is replaced by | |
| that are betaines | —NRR′R′′+, where R, R′ and R′′ are | |
| | members of the set of small alkyls, | |
| | ethyl, n-propyl, and so forth, | |
| | and where (for clarity) R, R′ and | |
| | R′′ may be the same or | |
| | dissimilar. | |
| All analogs where a | ROH is replaced by RSH, where | |
| phenolic (—OH) may | R is the indole substructure. | |
| be replaced by a | | |
| sulfhydryl (—SH) | | |
| All metal thiophenolates | —OH on the phenyl part of the | |
| | indole structure is replaced by | |
| | —SH, and the —SH is then reacted | |
| | with metal ion, displacing the | |
| | hydrogen. | |
| Sulfate analog to | [3-(2-Dimethylaminoethyl)- | |
| psilocybin (if feasible) | 1H-indol-4-yl] hydrogen sulfate | |
| Isotope Modified | | |
| All of the above | Deuterated versions | |
| | of the above molecules | |

In certain embodiments, the molecule is selected from Cannabigerol, Cannabichromene, Cannabidiol, Tetrahydrocannabinol, Cannabinol, Cannabieleoin, iso-Tetrahydrocannabinol, Cannabicyciol and derivatives thereof.

In certain embodiments, the molecule is cannabidiol or a derivative thereof. Exemplary compounds are set forth below:

Cannabidiol
5-(1,1-Dimethylheptyl)-2-(3-methyl-6-(1-
methylethenyl)-2-cyclohexen-1-yl)-1,3-
benzenediol
7-Hydroxycannabidiol
6alpha-Hydroxycannabidiol
2″-Hydroxycannabidiol
6beta-Hydroxycannabidiol
5″-Hydroxycannabidiol
3″-Hydroxycannabidiol
4″-Hydroxycannabidiol
1″-Hydroxycannabidiol
Ulipristal acetate
4-(3-Methyl-6-prop-1-en-2-ylcyclohex-
2-en-1-yl)-5-pentylbenzene-1,3-diol
5″-Hydroxycannabidiol glucoside
4-[(15,6S)-3-Methyl-6-prop-1-en-2-ylcyclohex-
2-en-1-yl]-5-pentylbenzene-1,3-diol
5-(2-Methyloctan-2-yl)-2-(3-methyl-6-prop-
1-en-2-ylcyclohex-2-en-1-yl)benzene-1,3-diol
Resorcinol, 4-p-mentha-1,8-dien-3-yl-5-pentyl-
4″-Hydroxycannabidiol glucoside
Cannabidiol-aldehyde diacetate
Cannabidivarin
1,3-Benzenediol, 2-[3-methyl-6-(1-methylethenyl)-
2-cyclohexen-1-yl]-5-pentyl-, (1R-
trans)-
Resorcinol, 2-p-mentha-1,8-dien-3-yl-5-pentyl-, (-)-(E)-
Trichloroisocyanuric acid

In certain embodiments, the molecule is set forth in the table below:

Cannabidiol
Olivetol
5-(Pentadeca-8,11,14-trien-1-yl)resorcinol
Cannabidivarin
5-Pentadecylresorcinol
3,5-Dihydroxy-4-isopropylstilbene
Cannabigerol
5-Heptadecylbenzene-1,3-diol
Grevillol
5-Nonadecylbenzene-1,3-diol
5-Henicosylbenzene-1,3-diol
5-Methyl-4-((1R,6R)-3-methyl-6-(1-methylethenyl)-
2-cyclohexen-1-yl)-1,3-benzenediol
1,3-Benzenediol, 2-[3-methyl-6-(1-methylethenyl)-
2-cyclohexen-1-yl]-5-pentyl-, (1R-trans)-
5-(Z-Heptadec-8-enyl) resorcinol
5-(8,11-Pentadecadienyl)-1,3-benzenediol
5-(8-Pentadecenyl)-1,3-benzenediol
Grifolin
Bilobol
5-Tricosyl-1,3-benzenediol
Abn-cbd
Cylindrocyclophane A
1,3-Benzenediol, 2-((2E)-3,7-dimethyl-2,6-octadienyl)-
5-((1E)-2-phenylethenyl)-
Demethylbatatasin IV
2-n-Hexyl-5-n-propylresorcinol
5-Pentacosylbenzene-1,3-diol
2,5-Dimethylresorcinol
Sesquicannabigerol
4-[2-[3,5-Dihydroxy-4-[(2E)-3,7-dimethyl-
2,6-octadienyl]phenyl]ethenyl]phenol
5-Undecylbenzene-1,3-diol
Neogrifolin
4-(3-Methyl-6-prop-1-en-2-ylcyclohex-2-en-1-
yl)-5-pentylbenzene-1,3-diol
2-Methyl-5-(8-pentadecenyl)-1,3-benzenediol
1,3-Benzenediol, 5-(8,11,14-heptadecatrienyl)-, (Z,Z,Z)-
4-Prenylresveratrol
4′-Prenyloxyresveratrol
Chlorophorin
5-Trideca-4,7-dienyl-benzene-1,3-diol
4-Prenyldihydropinosylvin
Resorstatin
4-Cyclohexylresorcinol
5-Heptylresorcinol
1,3-Benzenediol, 2-(3-methyl-6-(1-methylethenyl)-
2-cyclohexen-1-yl)-5-pentyl-, (1R-trans)-
(2R,8S,13R,19R)-8-Butyl-19-(4-hydroxybutyl)-2,13-
dimethyltricyclo[18.2.2.29,12]hexacosa-
1(22),9,11,20,23,25-hexaene-
10,21,24,26-tetrol
Merocyclophane D
5″-Hydroxycannabidiol
Ferruginene C
5-Desoxy-delta9-tetrahydrocannabinol
Schweinfurthin J
(Z)-5-(Heptadec-10-en-1-yl)resorcinol
2-((R)-6-Isopropenyl-3-methyl-cyclohex-2-enyl)-
5-pentyl-benzene-1,3-diol
2-((6S)-6-Isopropyl-3-methylcyclohex-2-enyl)-5-(2-
methyloctan-2-yl)benzene-1,3-diol
2-(3-Methylphenyl)-5-(2-phenylpropan-2-yl)benzene-1,3-diol
2-Cyclohexyl-5-(2-methyloctan-2-yl)benzene-1,3-diol
4-Methyl-5-pentylbenzene-1,3-diol
2,4,5-Trimethylbenzene-1,3-diol
5-Butylbenzene-1,3-diol
5-((8Z,11Z)-Heptadeca-8,11-dien-1-yl)resorcinol
Irisresorcinol
5-Ethylbenzene-1,3-diol
1,3-Benzenediol, 2-hexyl-5-methyl-
Arachidin-3
7-Hydroxycannabidiol
2-(3-Methylbut-2-enyl)-5-[(E)-2-phenylethenyl]benzene-1,3-diol
Cardoltriene
5-(Heptadec-12-enyl)resorcinol
5-Propylbenzene-1,3-diol
2-Hexyl-1,3-benzenediol
2,5-Dibutylbenzene-1,3-diol
4-Isoamylresorcinol
2-Methyl-5-pentadecylbenzene-1,3-diol
Stemphol
Cannabidiol-3-monomethyl ether
5-(1,1-Dimethylheptyl)resorcinol
4-Isopropylresorcinol
5-Heptacosylresorcinol
5-[(E)-2-(3,5-Difluorophenyl)ethenyl]-2-[(2E)-3,7-
dimethylocta-2,6-dienyl]benzene-1,3-diol
Merocyclophane A
Cannabigerovarin
4″-Hydroxycannabidiol
6beta-Hydroxycannabidiol
6alpha-Hydroxycannabidiol
5-[(E)-2-(4-Hydroxy-3-pentan-3-
ylphenyl)ethenyl]benzene-1,3-diol
5-[(1E,3E)-4-(4-Hydroxy-3-pentan-3-ylphenyl)buta-
1,3-dienyl]benzene-1,3-diol
5-[(1E,3E)-4-[4-Hydroxy-3,5-di(pentan-3-
yl)phenyl]buta-1,3-dienyl]benzene-1,3-diol
5-[(E)-2-[4-Hydroxy-3,5-di(pentan-3-
yl)phenyl]ethenyl]benzene-1,3-diol
5-(1-Hexyl-cyclopropyl)-2-((R)-6-isopropenyl-3-
methyl-cyclohex-2-enyl)-benzene-1,3-diol
1-[3,5-Dihydroxy-4-((R)-6-isopropenyl-3-methyl-
cyclohex-2-enyl)-phenyl]-cyclohexanecarbonitrile
1-[3,5-Dihydroxy-4-((R)-6-isopropenyl-3-methyl-cyclohex-
2-enyl)-phenyl]-cyclopentanecarbonitrile
5-(4-Methylphenyl)-2-[(1R)-3-methyl-6-prop-1-en-
2-ylcyclohex-2-en-1-yl]benzene-1,3-diol
5-(4-Methylphenyl)-2-[(1R,6R)-3-methyl-6-prop-1-en-
2-ylcyclohex-2-en-1-yl]benzene-1,3-diol
1-[3,5-Dihydroxy-4-[(1R,6R)-3-methyl-6-prop-1-en-2-
ylcyclohex-2-en-1-yl]phenyl]cyclopentane-1-carbonitrile
1-[3,5-Dihydroxy-4-[(1R,6R)-3-methyl-6-prop-1-en-2-
ylcyclohex-2-en-1-yl]phenyl]cyclohexane-1-carbonitrile
2-(3-Methyl-6-isopropenyl-2-cyclohexenyl)-
5-(1-hexylcyclopropyl)resorcinol
5-(2-Methyloctan-2-yl)-2-[(1R,2R)-4,6,6-trimethyl-2-
bicyclo[3.1.1]hept-3-enyl]benzene-1,3-diol
2-[(E)-3-Methylbut-1-enyl]-5-[(E)-2-phenylethenyl]benzene-1,3-diol
2-((2S)-2-Isopropyl-5-methylcyclohexyl)-5-pentylbenzene-1,3-diol
5-Icosylbenzene-1,3-diol
5-Tert-Butylresorcinol
5-Nonylbenzene-1,3-diol
2-(3,7-Dimethyl-2,6-octadienyl)-5-phenethylresorcinol
2-Ethyl-5-methylbenzene-1,3-diol
5-(2-Methyloctan-2-yl)-2-[(2S)-5-methyl-2-
propan-2-ylcyclohexyl]benzene-1,3-diol
Cylindrocyclophane F
4-(6-Isopropenyl-3-methylcyclohex-2-enyl)-5-methylbenzene-1,3-diol
2-(5-Methyl-2-prop-1-en-2-ylphenyl)-5-pentylbenzene-1,3-diol
2-Butyl-5-propyl-1,3-benzenediol
Climacostol
2-(3,5-Dimethylphenyl)-5-(2-methyloctan-2-yl)benzene-1,3-diol
Stilbostemin B
Piperogalin
Nostocyclyne A
5-(2-Methyloctan-2-yl)-2-[(6R)-3-methyl-6-prop-1-
en-2-ylcyclohex-2-en-1-yl]benzene-1,3-diol
1,3-Benzenediol, 2-methyl-5-pentyl-
1,3-Benzenediol, 2-ethyl-5-pentyl-
1,3-Benzenediol, 2-methyl-5-(8-tridecenyl)-, (Z)-
(E)-2-Ethyl-5-styrylbenzene-1,3-diol
Resorcinol, 4-p-mentha-1,8-dien-3-yl-5-pentyl-
2-Isopropylresorcinol
Resorcinol, 2-p-mentha-1,8-dien-3-yl-5-pentyl-, (-)-(E)-
Glepidotin C
Resorcinin
5-Pentadeca-8,11,14-trienylbenzene-1,3-diol
5-(2-Phenylethenyl)-2-i-propyl-1,3-benzenediol
2-Ethyl-5-(2-phenylethenyl)benzene-1,3-diol
Adipostatin B
5-(1,1-Dimethylheptyl)-2-(3-methyl-6-(1-methylethenyl)-
2-cyclohexen-1-yl)-1,3-benzenediol
1,3-Benzenediol, 4-methyl-5-pentadecyl-
2-[3-Methyl-6-(prop-1-en-2-yl)cyclohex-3-
en-1-yl]-5-pentylbenzene-1,3-diol
(2R,8R,13R,19R)-8-Butyl-19-(4-hydroxybutyl)-2,13-
dimethyltricyclo[18.2.2.29,12]hexacosa-1(22),9,11,20,23,25-hexaene-
10,21,24,26-tetrol
5-[(E)-2-(4-Hydroxyphenyl)ethenyl]-2-[(1E)-3-
methylbuta-1,3-dienyl]benzene-1,3-diol
2-[(E)-2-[3,5-Dihydroxy-4-(3-methylbut-2-
enyl)phenyl]ethenyl]benzene-1,3,5-triol
5-(12,15-Heneicosadienyl)-1,3-benzenediol
5-(14-Heneicosenyl)-1,3-benzenediol
5-(12-Heneicosenyl)-1,3-benzenediol
5-(10,13-Nonadecadienyl)-1,3-benzenediol
5-(10-Nonadecenyl)-1,3-benzenediol
2-Methyl-5-(8,11-pentadecadienyl)-1,3-benzenediol
(Z,Z)-2-Methyl-5-(8,11,14-pentadecatrienyl)-1,3-benzenediol
5-(2-Heptadecenyl)-1,3-benzenediol
5-(12-Heptadecenyl)-1,3-benzenediol
5-[(4-Hydroxypheny)ethenyl]-2-(3-methyl-
1-butenyl)-1,3-benzenediol
5-(2-Ethoxyethyl)-2-[(1R,6R)-3-methyl-6-prop-1-en-
2-ylcyclohex-2-en-1-yl]benzene-1,3-diol
(Z)-5-(Heptadec-2-en-1-yl)resorcinol
3″-Hydroxycannabidiol
2″-Hydroxycannabidiol
1″-Hydroxycannabidiol
5-(12-Nonadecenyl)resorcinol
5-(14-Nonadecenyl)resorcinol
5-(16-Heneicosenyl)-1,3-benzenediol
(E)-Arachidin II
5-((Z)-Pentadec-10-en-1-yl)resorcinol
5-(1,1,2,2-Tetradeuteriononadecyl)benzene-1,3-diol
2-[2-Hydroxy-3-methylidene-5-[(1S,6S)-2,2,6-
trimethylcyclohexyl]pentyl]-5-methylbenzene-1,3-diol
4-[(1S,6S)-3-Methyl-6-prop-1-en-2-ylcyclohex-
2-en-1-yl]-5-pentylbenzene-1,3-diol
4-[(2E)-3,7-Dimethyl-2,6-octadienyl]-5-pentyl-1,3-benzenediol
2-Methyl-5-[(E)-2-phenylethenyl]benzene-1,3-diol
2-[(2E)-3,7-Dimethylocta-2,6-dienyl]-5-[(E)-2-[4-
(trifluoromethyl)phenyl]ethenyl]benzene-1,3-diol
2-[(2E)-3,7-Dimethylocta-2,6-dienyl]-5-[(E)-2-(4-
fluorophenyl)ethenyl]benzene-1,3-diol
2-((6S)-6-Isopropyl-3-methylcyclohex-2-
enyl)-5-pentylbenzene-1,3-diol
2-[(1R,2R,5S)-5-Methyl-2-prop-1-en-2-
ylcyclohexyl]-5-pentylbenzene-1,3-diol
Polygonocinol
2-Octadecylbenzene-1,3-diol
4-(1-Phenylpropan-2-yl)benzene-1,3-diol
5-Dodecylbenzene-1,3-diol
5-Methyl-2-[(1R,6R)-3-methyl-6-prop-1-en-2-
ylcyclohex-2-en-1-yl]benzene-1,3-diol
2-Nonylbenzene-1,3-diol
2-Pentylbenzene-1,3-diol
2-Methyl-5-[(8Z,11Z)-pentadeca-8,11,14-trienyl]benzene-1,3-diol
5-Ethyl-2-methylbenzene-1,3-diol
3,5-Dihydroxy-1-isopropylbenzene
2-(1-Phenylethyl)benzene-1,3-diol
5-((Z)-Undec-3-en-1-yl)resorcinol
5-[(3Z,5Z)-Nona-3,5-dienyl]benzene-1,3-diol
5-[(E)-2-(2,4-Dihydroxyphenyl)ethenyl]-2-[(1E)-
3-methylbut-1-en-1-yl]benzene-1,3-diol
5-((Z)-Tridec-8-en-1-yl)resorcinol
2-Methyl-5-[(8Z,11Z)-pentadeca-8,11-dienyl]benzene-1,3-diol
5-[(4E,7E)-Trideca-4,7-dienyl]resorcinol
Dihydrostilbene base + 3O, 2Prenyl
1,3-Benzenediol, 2-[5-methyl-2-(1-methylethyl)cyclohexyl]-5-pentyl-
5-(3-Methyloctan-2-yl)benzene-1,3-diol
1,3-Benzenediol, 5-hexyl-
[1,1′-Biphenyl]-2,3′, 6-triol
4,6-Bis(1-methylpentyl)resorcinol
2-Methyl-5-propan-2-ylphenol; hydrate
5-[(E)-2-(4-Hydroxyphenyl)ethenyl]-2-[(2,6,6-
trimethylcyclohex-2-en-1-yl)methyl]benzene-1,3-diol
4-(2,4-Dihydroxy-3,6-dimethylphenyl)-
2,5-dimethylbenzene-1,3-diol
Tridecenylresorcinol
Pentacosylresorcinol
Heneicosylresorcinol
2-Methyl-5-pentadec-1-enylbenzene-1,3-diol
Heptadecylresorcinol
Trimethylresveratrol
Isopentenylresveratrol
5-(2-Hydroxyphenyl)benzene-1,3-diol
2-(2,4,4-Trimethylpentan-2-yl)benzene-1,3-diol
2-[(1R,6R)-3-Methyl-6-prop-1-en-2-ylcyclohex-2-en-1-
yl]-5-(oxetan-3-ylmethyl)benzene-1,3-diol
5-Pentyl-4-propylbenzene-1,3-diol
CID 127026868
5-((8Z,11Z)-Henicosa-8,11-dien-1-yl)resorcinol
5-((Z)-Henicos-14-en-1-yl)resorcinol
5-((Z)-Henicos-8-en-1-yl)resorcinol
5-((8Z,11Z)-Nonadeca-8,11-dien-1-yl)resorcinol
5-((Z)-Nonadec-8-en-1-yl)resorcinol
5-(10-Pentadecenyl)resorcinol
Tricosylresorcinol
5-(Hydroxymethyl)-2-(3-methylbut-2-enyl)benzene-1,3-diol
5-(Hydroxymethyl)-2-(3-hydroxy-3-methylbutyl)benzene-1,3-diol
5-[(E)-Tridec-1-enyl]benzene-1,3-diol
Decylresorcinol
CID 117679445
5-Pentadeca-1,3,5-trienylbenzene-1,3-diol
Nonadecylresorcin
2-Pentadeca-1,3,5-trienylbenzene-1,3-diol
5-Methyl-4-[(1R)-3-methyl-6-prop-1-en-2-
ylcyclohex-2-en-1-yl]benzene-1,3-diol
2-Pentadeca-1,3,5-trien-5-ylbenzene-1,3-diol
5-Octadecylresorcinol
5-Nonacosylresorcinol
2,4-Bis(3-methylbut-2-enyl)-5-(2-phenylethenyl)benzene-1,3-diol
5-[(E)-2-(4-Tert-butylphenyl)ethenyl]benzene-1,3-diol
2,4,5-Tripropylbenzene-1,3-diol
5-(Trideca-4,7-dien-1-YL)benzene-1,3-diol
5-(Heptadec-10-en-1-yl)benzene-1,3-diol
1,3-Benzenediol, 4-[(2E)-3,7-dimethyl-
2,6-octadienyl]-5-(2-phenylethyl)-
1,3-Benzenediol, 5-(1,1-dimethylheptyl)-
2-(3-methyl-2-cyclohexen-1-yl)-
1,3-Benzenediol, 5-(tridecenyl)-
2-Pentan-2-ylbenzene-1,3-diol
5-Methyl-2-propan-2-ylphenol; hydrate
5-{2-[4-(Propan-2-yl)phenyl]ethenyl}benzene-1,3-diol
5-[2-(4-Ethylphenyl)ethenyl]benzene-1,3-diol
5-Methyl-2-[(1R)-3-methyl-6-prop-1-en-2-
ylcyclohex-2-en-1-yl]benzene-1,3-diol
5-Methyl-2-(3-methylbut-2-en-1-yl)benzene-1,3-diol
2,2′-Methylenebis(5-butylphenol)
3′,5′-Dimethyl-4-(2-methyloctan-2-yl)[1,1′-biphenyl]-2,4′, 6-triol
5-(2-Methylhexan-2-YL)benzene-1,3-diol
4-[1-(2,4-Dihydroxyphenyl)hexyl]benzene-1,3-diol
2-(3-Methylbut-2-enyl)-5-pentylbenzene-1,3-diol
Tridecylresorcinol
5-Decylresorcinol
2,4-Bis(3-methylbut-2-enyl)-5-(2-phenylethyl)benzene-1,3-diol
Cylindrocyclophane C
2-[(1S,6S)-3-Methyl-6-prop-1-en-2-ylcyclohex-
2-en-1-yl]-5-propylbenzene-1,3-diol
2,4-Bis(3-methylbut-2-enyl)-5-pentylbenzene-1,3-diol
5-(10-Phenyldecyl)benzene-1,3-diol
5-(2-Methyloctan-2-yl)-2-[(2R)-5-methyl-2-prop-
1-en-2-ylcyclohexyl]benzene-1,3-diol
4-[3-(2,4-Dihydroxyphenyl)butan-2-yl]benzene-1,3-diol
5-[4-(3,5-Dihydroxyphenyl)hexan-3-yl]benzene-1,3-diol
2-Ethyl-5-[4-(4-ethyl-3-hydroxyphenyl)hexan-3-yl]phenol
5-[4-(3-Hydroxy-4-methylphenyl)hexan-3-yl]-2-methylphenol
1,3-Benzenediol, 4-[(6R)-3-methyl-6-(1-
methylethenyl)-2-cyclohexen-1-yl]-5-pentyl-
5-[(2Z,8Z,11Z)-Pentadeca-2,8,11-trienyl]benzene-1,3-diol
5-((5Z,8Z,11Z)-Heptadeca-5,8,11-trien-1-yl)resorcinol
2-Isopentyl-resorcinol
4-(3,5-Dimethylcyclohexyl)benzene-1,3-diol
Pentadecylresorcinol
4′-Methyl-1,1′-biphenyl-3,5-diol
Dodecylorcinol
5-(3-Methylundecan-2-YL)benzene-1,3-diol
5-Tetradecylbenzene-1,3-diol
5-(2-Methyloctan-2-yl)-2-(3-methyl-6-prop-1-
en-2-ylcyclohex-2-en-1-yl)benzene-1,3-diol
5-Methyl-2-(3-methyl-6-prop-1-en-2-
ylcyclohex-2-en-1-yl)benzene-1,3-diol
Triathylresorcin
5-(5-Phenylpentan-2-yl)benzene-1,3-diol
5-(Octan-2-YL)benzene-1,3-diol
5-[5-(4-Fluorophenyl)pentan-2-yl]benzene-1,3-diol
2-Propan-2-ylbenzene-1,3,5-triol
2-Dodecylbenzene-1,3-diol
5-((3Z,6Z,9Z,12Z,15Z,18Z)-Henicosa-
3,6,9,12,15,18-hexaen-1-yl)resorcinol
5-((4Z,7Z,10Z,13Z,16Z)-Nonadeca-
4,7,10,13,16-pentaen-1-yl)resorcinol
5-((7Z,10Z,13Z,16Z)-Nonadeca-7,10,13,16-tetraen-1-yl)resorcinol
5-((Z)-Pentadec-6-en-1-yl)resorcinol
5-((Z)-Pentadec-4-en-1-yl)resorcinol
5-((5Z,8Z,11Z,14Z)-Heptadeca-5,8,11,14-tetraen-1-yl)resorcinol
2-Methyl-5-nonylbenzene-1,3-diol
4-[(2,4-Dihydroxy-6-propylphenyl)methyl]-5-propylbenzene-1,3-diol
2-[(1S,2S,5S)-4-(Hydroxymethyl)-6,6-dimethyl-2-
bicyclo[3.1.1]hept-3-enyl]-5-(2-methyloctan-2-yl)benzene-1,3-diol
2-Octylresorcinol
2-Heptylbenzene-1,3-diol
4-(3-Methyl-2-butenyl)-5-phenethylbenzene-1,3-diol
5-[3,5-Dihydroxy-4-[(1R,6R)-3-methyl-6-prop-1-
en-2-ylcyclohex-2-en-1-yl]phenyl]pentanoic acid
2-Cyclohexyl-benzene-1,3-diol
2-Butyl-5-methylbenzene-1,3-diol
Resorcinol, 4,4′-(1,2-diethylethylene)di-
2-Tert-butyl-5-methylbenzene-1,3-diol
2-(4-Methylcyclohexyl)benzene-1,3-diol
5-[3-(3,5-Dihydroxyphenyl)-2,3-dimethylbutan-2-yl]benzene-1,3-diol
5-(2-Cyclohexylpropan-2-yl)benzene-1,3-diol
2-[(1R,6R)-3-Methyl-6-(1-methylethenyl)-2-
cyclohexen-1-yl]-5-pentyl-1,3-benzenediol
1-Isopentyl-2,4,6-trihydroxybenzene
2-(3-Methyl-2-butenyl)-5-[2-(4-hydroxyphenyl)ethyl]-1,3-benzenediol
5-(1-Hexyl-cyclopentyl)resorcinol
2-(2-Methylpropyl)benzene-1,3-diol
1,3-Benzenediol, 5-hexadecyl-
Machaeridiol A
2-(3,3-Dimethylcyclohexyl)-5-(2-methyloctan-2-yl)benzene-1,3-diol
Machaeridiol B
4,6-Diisopropylresorcinol
5-Methyl-2-[(6R)-3-methyl-6-prop-1-en-2-
ylcyclohex-2-en-1-yl]benzene-1,3-diol
2-[(6S)-3-Methyl-6-prop-1-en-2-ylcyclohex-
2-en-1-yl]-5-pentylbenzene-1,3-diol
1,3-Benzenediol, 5-(2-heptadecenyl)-, (Z)-
2-[(E)-Pentadec-5-en-8-yl]benzene-1,3-diol
1,3-Benzenediol, 5-(8Z,11Z)-8,11-pentadecadienyl-
1,3-Benzenediol, 5-(nonadecenyl)-
5-Pentadec-8-enylbenzene-1,3-diol
Stilbostemin C
4,4′-(1,2-Diethylethylene)diresorcinol
1,3-Benzenediol, 5-methyl-2-(3,7,11-trimethyl-
2,6,10-dodecatrienyl)-
5-Heptadec-12-enylbenzene-1,3-diol
2-(3,7-Dimethyloctyl)-5-methylbenzene-1,3-diol
2-[3-Methyl-6-(1-methylethylidene)-2-
cyclohexen-1-YL]-1,3-benzenediol
4-[1-(2,4-Dihydroxyphenyl)butyl]benzene-1,3-diol
5-[2-(2,4-Dihydroxyphenyl)ethenyl]-2-(3,7-
dimethylocta-2,6-dienyl)benzene-1,3-diol
1,3-Benzenediol, 2-(3,7-dimethyl-2,6-octadienyl)-5-pentyl-, (E)-
2-(3-Methyl-6-prop-1-en-2-ylcyclohex-2-
en-1-yl)-5-propylbenzene-1,3-diol
4-[(4-Hydroxy-2,5-dimethylphenyl)methyl]-
2-methylbenzene-1,3-diol
(3-Ethyl-5-hydroxyphenyl)oxidanium
5-(4-Phenylbutyl)benzene-1,3-diol
2-[6-(2-Hydroxypropan-2-yl)-3,4-dimethylcyclohex-
3-en-1-yl]-5-pentylbenzene-1,3-diol
2-[5-(2-Hydroxypropan-2-yl)cyclohexen-1-yl]-
5-(3-methyloctan-2-yl)benzene-1,3-diol
5-Butyl-2-cyclohex-3-en-1-ylbenzene-1,3-diol
4-[1-(2,4-Dihydroxyphenyl)decyl]benzene-1,3-diol
4-[(4-Pentan-2-ylphenyl)methyl]benzene-1,3-diol
5-(2-Phenylethenyl)-4-propan-2-ylbenzene-1,3-diol
2-[[4-(2-Methylpentan-2-yl)phenyl]methyl]benzene-1,3-diol
5-(3-Methyloctan-2-yl)-2-(4,6,6-trimethyl-2-
bicyclo[3.1.1]hept-3-enyl)benzene-1,3-diol
2-(2-Cyclohexylethyl)benzene-1,3-diol
5-(3,3-Dimethyloctan-2-yl)benzene-1,3-diol
4-[(4-Propan-2-ylphenyl)methyl]benzene-1,3-diol
2-Cyclohex-3-en-1-yl-5-pentylbenzene-1,3-diol
2-(Cyclohexen-1-yl)-5-pentylbenzene-1,3-diol
5-(2-Methyloctyl)benzene-1,3-diol
2,4-Di(hexan-2-yl)benzene-1,3-diol
4-(4-Hydroxy-2,3,5-trimethylphenyl)-2-methylbenzene-1,3-diol
5-Oct-6-en-2-ylbenzene-1,3-diol
2-[1-(2-Hydroxy-4-methylphenyl)-2-methylpropyl]-5-methylphenol
2-[6-(2-Hydroxypropan-2-yl)-3-methylcyclohex-
3-en-1-yl]-5-pentylbenzene-1,3-diol
5-(2,3-Dimethylnonan-2-yl)benzene-1,3-diol
5-(3,4-Dimethyloctan-2-yl)benzene-1,3-diol
2-[4-(2,6-Dihydroxy-4-methylphenyl)phenyl]-
5-methylbenzene-1,3-diol
5-(4-Methylnon-3-en-3-yl)benzene-1,3-diol
2,5-Didecylbenzene-1,3-diol
2-(2-Ethylhexyl)benzene-1,3-diol
5-Hexan-3-ylbenzene-1,3-diol
5-Octan-4-ylbenzene-1,3-diol
2-Octan-2-ylbenzene-1,3-diol
4-(5-Methyl-2-propan-2-ylcyclohexyl)-5-(2-
phenylethenyl)benzene-1,3-diol
5-[1-(3,5-Dihydroxy-4-methylphenyl)dodecyl]-
2-methylbenzene-1,3-diol
2-(4-Methyl-2-propan-2-ylphenyl)-5-pentylbenzene-1,3-diol
2-Dec-2-enylbenzene-1,3-diol
2-Cyclohept-2-en-1-ylbenzene-1,3-diol
2-Tetradecan-2-ylbenzene-1,3-diol
2-Ethyl-4,5-dimethylbenzene-1,3-diol
2-(3-Methylpentan-2-yl)benzene-1,3-diol
2-(3-Methylundecan-2-yl)benzene-1,3-diol
5-(113C)Pentyl(1,2,3,5,6-13C5)cyclohexa-1,3,5-triene-1,3-diol
5-Pentyl(313C)cyclohexa-1,3,5-triene-1,3-diol
2-[(1R)-3-Methylcyclohexyl]-5-(2-methyloctan-
2-yl)benzene-1,3-diol
5-Pentyl(1,2,3,6-13C4)cyclohexa-1,3,5-triene-1,3-diol
2-[(2S,5R)-5-Methyl-2-propan-2-ylcyclohexyl]-
5-pentylbenzene-1,3-diol
2-[6-[(Z)-But-2-en-2-yl]-3-methylcyclohex-2-en-1-yl]-
5-pentylbenzene-1,3-diol; ethane
2-(3,7-Dimethyloctyl)-5-propan-2-ylbenzene-1,3-diol
2-(3,7-Dimethyloctyl)-5-fluorobenzene-1,3-diol
5-Cyclopentyl-2-(3,7-dimethyloctyl)benzene-1,3-diol
2-(3,7-Dimethyloctyl)-5-(trifluoromethyl)benzene-1,3-diol
4,6-Dideuterio-2,5-bis(1,1,2,2,3,3,4,4,5,5,5-
undecadeuteriopentyl)benzene-1,3-diol
2-[(1R)-3-Methyl-6-prop-1-en-2-ylcyclohex-
2-en-1-yl]-5-pentylphenol
4-(3-Methylcyclohex-2-en-1-yl)-5-
pentylbenzene-1,3-diol; prop-1-ene
4-[(1S)-3-Methylcyclohex-2-en-1-yl]-5-
pentylbenzene-1,3-diol; prop-1-ene
Ethane; 2-[(1R)-3-methyl-6-prop-1-en-2-ylcyclohex-
2-en-1-yl]-5-pentylbenzene-1,3-diol
2-[(6S)-3-Ethyl-6-prop-1-en-2-ylcyclohex-3-
en-1-yl]-5-pentylbenzene-1,3-diol
Ethane; 5-ethyl-2-(3-methylcyclohex-2-
en-1-yl)benzene-1,3-diol; prop-1-ene
2-[(6S)-3-Methyl-6-prop-1-en-2-ylcyclohex-3-en-
1-yl]-5-propylbenzene-1,3-diol
2-[(1R,6R)-3-Ethyl-6-prop-1-en-2-ylcyclohex-3-
en-1-yl]-5-pentylbenzene-1,3-diol
2-[(6S)-3-Methyl-6-prop-1-en-2-ylcyclohex-3-en-
1-yl]-5-pentylbenzene-1,3-diol
2-[(1R)-3-(Hydroxymethyl)-6-prop-1-en-2-ylcyclohex-
2-en-1-yl]-5-pentylbenzene-1,3-diol
2-[(6S)-3-(Hydroxymethyl)-6-prop-1-en-2-ylcyclohex-
2-en-1-yl]-5-propylbenzene-1,3-diol
2-[(1S,6S)-6-Hydroxy-3-methyl-6-prop-1-en-2-
ylcyclohex-2-en-1-yl]-5-pentylbenzene-1,3-diol
2-[(1R,2R)-5-Methyl-2-prop-1-en-2-ylcyclohex-
3-en-1-yl]-5-pentylbenzene-1,3-diol
2-[(6S)-3-Methyl-6-prop-1-en-2-ylcyclohexen-1-
yl]methyl]-5-pentylbenzene-1,3-diol
2-[(1S,6S)-6-Hydroxy-3-(hydroxymethyl)-6-prop-1-
en-2-ylcyclohex-2-en-1-yl]-5-propylbenzene-1,3-diol
2-[(1R,6S)-3-Methyl-6-propan-2-ylcyclohex-3-
en-1-yl]-5-pentylbenzene-1,3-diol
2-[(6R)-6-Tert-butyl-3-methylcyclohex-2-
en-1-yl]-5-pentylbenzene-1,3-diol
5-[(E)-2-(2,4-Dihydroxyphenyl)ethenyl]-2-[(1E)-3-
methylbuta-1,3-dienyl]benzene-1,3-diol
2-[(6S)-3-Methyl-6-[(Z,2R)-pent-3-en-2-yl]cyclohex-
2-en-1-yl]-5-propylbenzene-1,3-diol
4-[(3S)-3,4-Dimethylpenta-1,4-dien-2-yl]-6-
propan-2-ylbenzene-1,3-diol
5-Methyl-2-[(1S,6S)-3-methyl-6-(3-methylbut-3-
enyl)cyclohex-2-en-1-yl]benzene-1,3-diol
5-[2-(4-Hydroxyphenyl)ethyl]-2-methylbenzene-1,3-diol
CID 152843908
2-[(1R,6R)-6-Buta-1,3-dien-2-yl-3-ethenylcyclohex-
2-en-1-yl]-5-hex-5-enylbenzene-1,3-diol
4-[3-Methyl-6-(3-methylbuta-1,3-dienyl)cyclohex-
2-en-1-yl]benzene-1,3-diol
3-Hydroxy-2-[(1R,6R)-3-methyl-6-prop-1-en-2-
ylcyclohex-3-en-1-yl]-5-pentylphenolate
5-Octadecan-8-ylbenzene-1,3-diol
2,5-Diphenylbenzene-1,3-diol
2-Cyclohexa-2,4-dien-1-ylbenzene-1,3,5-triol
2-[(2E)-3,7-Dimethylocta-2,6-dienyl]-3-hydroxy-5-propylphenolate
5-Pentyl-2-(1,1,2,2,3,3,4,4,5,5,5-
undecadeuteriopentyl)benzene-1,3-diol
2,5-Dipentylbenzene-1,3-diol
4-Ethyl-6-[(4-hydroxy-2,3,5-
trimethylphenyl)methyl]benzene-1,3-diol
2-[3-(2-Hydroxy-4-methylphenyl)butan-2-yl]-5-methylphenol
5-Nonyl-2-propan-2-ylphenol
2-Nonyl-5-propan-2-ylphenol
3-Azidooxy-5-methyl-2-propan-2-ylphenol
2,4,5-Trimethyl-6-(2,3,5-trimethylphenyl)benzene-1,3-diol
5-Methyl-4-(2-propylphenyl)benzene-1,3-diol
3-Hydroxy-2-[(1R,6R)-3-methyl-6-prop-1-en-
2-ylcyclohex-2-en-1-yl]-5-propylphenolate
2-Tert-butyl-5-cyclohex-2-en-1-ylbenzene-1,3-diol
2-(3,7-Dimethylocta-2,6-dienyl)-3-hydroxy-5-pentylphenolate
2-[(2E)-3,7-Dimethylocta-2,6-dienyl]-3-hydroxy-5-pentylphenolate
4-Benzyl-2-ethenyl-5-methylbenzene-1,3-diol
2,5-Bis(6-methylheptyl)benzene-1,3-diol
3-Hydroxy-2-[(1R,6R)-3-methyl-6-prop-1-en-
2-ylcyclohex-2-en-1-yl]-5-pentylphenolate
3-Hydroxy-2-(3-methyl-6-prop-1-en-2-ylcyclohex-
2-en-1-yl)-5-pentylphenolate
2-[3,7-Bis(sulfanyl)octa-2,6-dienyl]-5-phenylbenzene-1,3-diol
5-Methyl-4-(2-methyl-5-propan-2-ylphenyl)-
2-propan-2-ylbenzene-1,3-diol
5-Phenyl-4-propan-2-yl-2-propylbenzene-1,3-diol
4-[1-(4-Phenylphenyl)propan-2-yl]benzene-1,3-diol
3-Hydroxy-2-[(1R,6R)-3-methyl-6-prop-1-en-2-
ylcyclohex-3-en-1-yl]-5-propylphenolate
2-Tert-butyl-5-cyclopent-2-en-1-ylbenzene-1,3-diol
2,5-Dicyclohexylbenzene-1,3-diol
5-(1-Phenylbutyl)benzene-1,3-diol
2-(2-Methylpentan-3-yl)benzene-1,3-diol
5-(2-Diphenylphosphanylethyl)-2-propan-2-ylbenzene-1,3-diol
2,4,5-Tris-decylbenzene-1,3-diol
3-[3-(3,5-Dihydroxy-4-propan-2-ylphenyl)phenyl]prop-2-enenitrile
2-(2-Ethylpentyl)benzene-1,3,5-triol
4-(5-Hydroxy-2-propan-2-ylphenyl)benzene-1,3-diol
5-[(E)-2-(4-Hydroxyphenyl)ethenyl]-4-[(1R,6S)-3-methyl-
6-prop-1-en-2-ylcyclohex-2-en-1-yl]benzene-1,3-diol
5-(4-Methylpentyl)-2-undecan-2-ylbenzene-1,3-diol
2,5-Bis(2-methylbutan-2-yl)benzene-1,3-diol
5-[2-[4-(5,5-Dimethylheptyl)phenyl]ethyl]benzene-1,3-diol
4-[2-(6-Hydroxy-6-methylcyclohexa-2,4-dien-
1-yl)propan-2-yl]benzene-1,3-diol
4-Phenyl-2,5-di(propan-2-yl)benzene-1,3-diol
5-Tritriacontylbenzene-1,3-diol
5-Phenyl-2,4,6-tri(propan-2-yl)benzene-1,3-diol
4-(4-Propylcyclohexyl)benzene-1,3-diol
2-Cyclohexyl-5-(2-methylpropyl)phenol
3-Tert-butylperoxy-2-propan-2-ylphenol
5-[14-(3,5-Dihydroxyphenyl)tetradecyl]-2-methylbenzene-1,3-diol
2-(1,3-Diphenylbutyl)benzene-1,3-diol
2-(2-Methylidenecyclohexyl)benzene-1,3-diol
5-(3-Cyclobutylpropyl)benzene-1,3-diol
4-[2-(4-Hydroxycyclohexa-2,4-dien-1-yl)ethyl]benzene-1,3-diol
5-[2-(3,5-Dihydroxyphenyl)propyl]benzene-1,3-diol
6-Pentyl-5,6,7,8-tetrahydronaphthalene-1,3-diol
2-[4-Cyclohexyl-3-(hydroxymethyl)cyclohex-2-en-1-
yl]-5-(5-iodo-2-methylpentan-2-yl)benzene-1,3-diol
5-Methyl-2-[(1S,6S)-3-methyl-6-(2-
methylcyclopropyl)cyclohex-2-en-1-yl]benzene-1,3-diol
5-[(3R)-3-Ethyl-2-methyloctan-2-yl]benzene-1,3-diol
5-(4-Hydroxycyclohexa-1,3-dien-1-yl)benzene-1,3-diol
4-(4-Hydroxy-2,5-dimethylphenyl)-6-methylbenzene-1,3-diol
4-[Dimethyl(propan-2-yl)-lambda4-tellanyl]-
6-[(Z)-pent-3-en-2-yl]benzene-1,3-diol
2-[(1R,6S)-3,6-Dimethylcyclohex-2-en-
1-yl]-5-pentylbenzene-1,3-diol
5-[(E)-2-[4-(Cyclopropylmethyl)phenyl]ethenyl]benzene-1,3-diol
4-Butan-2-yl-6-tert-butyltellanylbenzene-1,3-diol
5-[2-(4-Propan-2-ylphenyl)ethyl]benzene-1,3-diol
4-Ethyl-6-propan-2-ylbenzene-1,3-diol
[3-[(E)-2-(4-Tert-butylphenyl)ethenyl]-5-hydroxyphenyl]oxidanium
2-[(6S)-3-(Hydroxymethyl)-6-prop-1-en-2-
ylcyclohex-3-en-1-yl]-5-propylbenzene-1,3-diol
4-[(6S)-1,3-Dimethyl-6-prop-1-en-2-
ylcyclohex-2-en-1-yl]benzene-1,3-diol
4-[(5-Cyclohexyl-4-hydroxy-2-
methylphenyl)methyl]benzene-1,3-diol
2-[(1R,6R)-6-[(2E,5Z)-5-[2-(2,6-Dihydroxy-
4-pentylphenyl)ethylidene]-
9-methyldeca-2,8-dien-2-yl]-3-methylcyclohex-2-en-1-yl]-5-
propylbenzene-1,3-diol
5-[(E)-2-(4-Ethylcyclohexa-2,4-dien-1-yl)ethenyl]benzene-1,3-diol
5-[(E)-2-(2-Hydroxy-4-methylphenyl)ethenyl]benzene-1,3-diol
5-[4-[(1S)-2-Methylcyclopropyl]butyl]-2-[(1R,6R)-3-
methyl-6-prop-1-en-2-ylcyclohex-2-en-1-yl]benzene-1,3-diol
5-[(E)-2-(4-Hydroxyphenyl)ethenyl]-2-[(1S)-3-
methylcyclohex-2-en-1-yl]benzene-1,3-diol
2-[(2E)-3,7-Dimethylocta-2,6-dienyl]-
5-(2-iodoethyl)benzene-1,3-diol
5-Methyl-4-pentan-3-ylbenzene-1,3-diol
5-(7-Phenylheptyl)benzene-1,3-diol
2-[(1R,2R)-5-Methyl-2-prop-1-en-2-ylcyclohexyl]-
5-propylbenzene-1,3-diol
2-[(2S,5R,7S)-4-Methyl-2-tricyclo[3.2.0.02,7]hept-
3-enyl]-5-pentylbenzene-1,3-diol
5-Methyl-2-[(2R)-4,7,7-trimethyl-2-
bicyclo[4.1.0]hept-3-enyl]benzene-1,3-diol
(3-Hydroxy-4-propan-2-ylphenyl)oxidanium
2-[(1R,6R)-3,6-Dimethyl-6-prop-1-en-2-
ylcyclohex-2-en-1-yl]-5-propylbenzene-1,3-diol
2-(6-Tert-butyl-2,3-dimethylphenyl)-5-hexylbenzene-1,3-diol
5-[(E)-2-[4-(Cyclobuten-1-yl)phenyl]ethenyl]benzene-1,3-diol
2-[(1S,6S)-3-Methyl-6-(3-methylbut-3-
enyl)cyclohex-2-en-1-yl]-5-propylbenzene-1,3-diol
5-Methanidyl-2-methylbenzene-1,3-diol; uranium
3-Methoxy-2-[(1S)-3-methylcyclohex-2-en-1-yl]-5-pentylphenol
2,5-Dimethyl-4-propylbenzene-1,3-diol
4-(2-Methylbutan-2-yltellanyl)-6-propan-2-ylbenzene-1,3-diol
3-Methoxy-2-[(1R)-3-methyl-6-prop-1-en-2-
ylcyclohex-2-en-1-yl]-5-pentylphenol
5-Methyl-2-(3-methyl-6-propan-2-ylcyclohexa-
2,4-dien-1-yl)benzene-1,3-diol
2-[(2S,5S,7S)-1,4-Dimethyl-2-tricyclo[3.2.0.02,7]hept-
3-enyl]-5-pentylbenzene-1,3-diol
2-[(1S,6S)-3-(Hydroxymethyl)-6-propan-2-
ylcyclohex-2-en-1-yl]-5-pentylbenzene-1,3-diol
5-Pentyl-2-(5-prop-1-en-2-yloctan-4-yl)benzene-1,3-diol
2-[(1S)-6-Ethenyl-3-methylcyclohex-2-en-
1-yl]-5-pentylbenzene-1,3-diol
5-[(5-Hydroxy-3,4-dimethylcyclohexa-
1,5-dien-1-yl)methyl]-2-methylphenol
(3-Hydroxy-2,5-dimethylphenyl)oxidanium
5-[(11E,16E)-16,18-Dihydroxy-14-methyl-15-
methylidenenonadeca-11,16,18-trienyl]benzene-1,3-diol
2-[(1S,6S)-1-Hydroxy-3-(hydroxymethyl)-6-prop-1-en-
2-ylcyclohex-2-en-1-yl]-5-[(E)-pent-1-enyl]benzene-1,3-diol
4-[2-[2-Hydroxy-3-[(2-hydroxy-4-methylphenyl)methyl]-
5-methylcyclohexa-1,3-dien-1-yl]ethyl]benzene-1,3-diol
5-[(E)-2-(4-Hydroxycyclohexa-1,3-dien-1-yl)ethenyl]-
2-[1-(4-hydroxyphenyl)ethyl]benzene-1,3-diol
5-Methyl-2-[(1S)-5-methyl-2-prop-1-en-2-
ylcyclohexyl]benzene-1,3-diol
2-[(2Z)-2-Fluoro-3,7-dimethylocta-2,6-
dienyl]-5-propylbenzene-1,3-diol
2-[(1S,6S)-3-Methyl-6-prop-1-en-2-ylcyclohept-
2-en-1-yl]-5-pentylbenzene-1,3-diol
2-[(1R,6R)-3-(Hydroxymethyl)-6-methylcyclohex-
3-en-1-yl]-5-pentylbenzene-1,3-diol
4-(3,4-Dimethylcyclohexyl)benzene-1,3-diol
2-[(1R,6R)-6-(2-Hydroxypropan-2-yl)-3-methylcyclohex-
2-en-1-yl]-5-methylbenzene-1,3-diol
5-[(E)-2-(4-Hydroxyphenyl)ethenyl]-4-(3-methyl-6-
propan-2-ylcyclohex-2-en-1-yl)benzene-1,3-diol
2-[(1S)-5-Methyl-2-propan-2-yliodanuidylcyclohexyl]-
5-pentylbenzene-1,3-diol
2-[(1R,6R)-3-Methyl-4-[(1S)-2-methylcyclopropyl]-6-
prop-1-en-2-ylcyclohex-2-en-1-yl]-5-pentylbenzene-1,3-diol
5-(5-Hydroxy-4-methylcyclohexa-2,4-dien-1-yl)-2-methylphenol
5-[(E)-2-(4-Hydroxy-6-methylcyclohexa-
1,3-dien-1-yl)ethenyl]benzene-1,3-diol
5-(3-Hydroxy-4-methylcyclohexa-2,4-dien-1-yl)-2-methylphenol
5-[(2S)-1-(4-Hydroxyphenyl)propan-2-yl]benzene-1,3-diol
2-[(6R)-3-Methyl-6-prop-1-en-2-ylcyclohexa-
1,3-dien-1-yl]-5-propylbenzene-1,3-diol
2-[(1R,6R)-3,6-Dimethyl-6-prop-1-en-2-ylcyclohex-
2-en-1-yl]-5-methylbenzene-1,3-diol
4-Butan-2-yl-6-(2-methylbutan-2-yltellanyl)benzene-1,3-diol
2-[(1S,6S)-3,6-Dimethyl-6-prop-1-en-2-ylcyclohex-
3-en-1-yl]-5-propylbenzene-1,3-diol
2-[(1R,2R)-4-Methyl-1-prop-1-en-2-yl-2-
bicyclo[4.1.0]hept-3-enyl]-5-pentylbenzene-1,3-diol
5-(3-Hydroxy-4-methylphenyl)-2,6-
dimethylcyclohexa-1,3-diene-1,3-diol
4-(3-Hydroxy-3-methylbutyl)-5-methylbenzene-1,3-diol
2-(2,2,5,5-Tetramethylhexan-3-yl)benzene-1,3,5-triol
4-[(1R,6R)-3-Methyl-6-prop-1-en-2-ylcyclohex-2-en-1-yl]-2-
[(1R,6S)-3-methyl-6-prop-1-en-2-ylcyclohex-
2-en-1-yl]benzene-1,3,5-triol
5-Fluoro-4-[(1S,6R)-3-methyl-6-prop-1-en-2-
ylcyclohex-2-en-1-yl]benzene-1,3-diol
5-Fluoro-2-[(1S,6R)-3-methyl-6-prop-1-en-2-
ylcyclohex-2-en-1-yl]benzene-1,3-diol
5-Ethynyl-4-[(1S,6R)-3-methyl-6-prop-1-en-2-
ylcyclohex-2-en-1-yl]benzene-1,3-diol
2,4-Bis[(2E)-3,7-dimethylocta-2,6-dienyl]-5-pentylbenzene-1,3-diol
5-[(1Z)-4,8-Dimethylnona-1,7-dienyl]benzene-1,3-diol
5-[(1E)-3,7-Dimethylocta-1,6-dienyl]benzene-1,3-diol
5-[(1E)-4,8-Dimethylnona-1,7-dienyl]benzene-1,3-diol
4-[2-(4-Methylphenyl)ethyl]benzene-1,3-diol
4-[1-(4-Methylphenyl)hexyl]benzene-1,3-diol
4-[1-(4-Methylphenyl)ethyl]benzene-1,3-diol
4-[1-(4-Methylphenyl)propyl]benzene-1,3-diol
4-[1-(4-Methylphenyl)butyl]benzene-1,3-diol
2-(3-Methyl-5-prop-1-en-2-ylcyclopent-2-en-
1-yl)-5-pentylbenzene-1,3-diol
2-[(1S,5S)-3-Methyl-5-prop-1-en-2-ylcyclopent-
2-en-1-yl]-5-pentylbenzene-1,3-diol
2-[(4S,5Z)-2,6-Dimethylnona-1,5-dien-4-yl]-
5-pentylbenzene-1,3-diol
5-[2-[(1R,5R)-3-Methyl-5-prop-1-en-2-ylcyclopent-
2-en-1-yl]pentyl]benzene-1,3-diol
5-(2-lodo-2,3-dimethylpentan-3-yl)-2-[(1R,6R)-3-
methyl-6-prop-1-en-2-ylcyclohex-2-en-1-yl]benzene-1,3-diol
2-[(6S)-3-(Aminomethyl)-6-prop-1-en-2-ylcyclohex-
2-en-1-yl]-5-pentylbenzene-1,3-diol
Potassium; 3-hydroxy-2-[(1R,6R)-3-methyl-6-prop-
1-en-2-ylcyclohex-2-en-1-yl]-5-pentylphenolate
Sodium; 3-hydroxy-2-[(1R,6R)-3-methyl-6-prop-
1-en-2-ylcyclohex-2-en-1-yl]-5-pentylphenolate
2-[(1R)-5-Methyl-2-prop-1-en-2-ylcyclohex-
2-en-1-yl]-5-pentylbenzene-1,3-diol
5-Pentyl-2-[(1R)-2-prop-1-en-2-ylcyclohex-
2-en-1-yl]benzene-1,3-diol
2-[(1R)-3-(Hydroxymethyl)-6-prop-1-en-2-
ylcyclohex-2-en-1-yl]-5-propylbenzene-1,3-diol
2-(lodomethyl)-5-methylbenzene-1,3-diol
2-[[(1R,6R)-3-Methyl-6-prop-1-en-2-ylcyclohex-
2-en-1-yl]methyl]-5-pentylbenzene-1,3-diol
4-[2-(4-Hydroxyphenyl)butyl]benzene-1,3-diol
5-[(2E)-4-Hydroxy-3-propan-2-ylpenta-
2,4-dien-2-yl]-2-propan-2-ylphenol
5-(5,5-Dimethylhexan-3-yl)benzene-1,3-diol
Acetylene; 2-[(6R)-3-methyl-6-prop-1-en-2-
ylcyclohex-2-en-1-yl]-5-pentylbenzene-1,3-diol
Ethane; 5-methyl-2-(3-methyl-6-prop-1-en-2-
ylcyclohex-2-en-1-yl)benzene-1,3-diol
Ethane; 5-methyl-2-[(Z)-5-methyloct-4-
en-3-yl]benzene-1,3-diol; prop-2-enoic acid
4-But-1-en-2-yl-5-pentylbenzene-1,3-diol
Ethane; 5-ethylbenzene-1,3-diol
2-[[(1S,6S)-3-Methyl-6-propan-2-ylcyclohex-2-en-
1-yl]methyl]-5-pentylbenzene-1,3-diol
5-Pentyl-2-phenylbenzene-1,3-diol
2-(3-Methyl-6-prop-1-en-2-ylcyclohex-2-en-1-yl)-5-propylphenol
5-(4-Butyl-3,5-dihydroxyphenyl)pentanethial
5-[4-(4-Hexylcyclohexyl)phenyl]benzene-1,3-diol
(4Z)-2,5-Dimethylocta-1,4-diene; 5-methylbenzene-1,3-diol
(5Z)-2,5-Dimethylocta-1,5-diene; 5-methylbenzene-1,3-diol
Ethene; 5-methyl-2-(3-methylcyclohex-2-en-1-yl)benzene-1,3-diol
5-Methyl-2-(3-methylcyclohex-3-en-1-yl)benzene-1,3-diol
5-Methyl-2-(3-methylcyclohex-3-en-1-
yl)benzene-1,3-diol; prop-1-ene
2-(3-Fluorocyclohex-2-en-1-yl)-5-pentylbenzene-1,3-diol; propane
2-(4-Fluoro-3-methylcyclohex-2-en-1-yl)-
5-pentylbenzene-1,3-diol; propane
2-(4-Hydroxy-3-methylcyclohex-2-en-1-yl)-
5-pentylbenzene-1,3-diol; propane
2-(4-Fluoro-3-methylcyclohex-2-en-1-yl)-
5-(2-methyloctan-2-yl)benzene-1,3-diol; propane
2-(3-Methylcyclohex-2-en-1-yl)-5-(2-methyloctan-
2-yl)benzene-1,3-diol; propane
2-(4-Hydroxy-3-methylcyclohex-2-en-1-yl)-5-
(2-methyloctan-2-yl)benzene-1,3-diol; propane
2-[3-(Hydroxymethyl)cyclohex-2-en-1-yl]-
5-pentylbenzene-1,3-diol; propane
2-[3-(Fluoromethyl)cyclohex-2-en-1-yl]-5-
(2-methyloctan-2-yl)benzene-1,3-diol; propane
4-Fluoro-5-methyl-2-(3-methylcyclohex-2-en-1-
yl)benzene-1,3-diol; 2-methylprop-1-ene
Fluoroethene; 5-methyl-2-(3-
methylcyclohex-2-en-1-yl)benzene-1,3-diol
2-[3-(Hydroxymethyl)cyclohex-2-en-1-yl]-5-
(2-methyloctan-2-yl)benzene-1,3-diol; propane
Ethane; 5-propan-2-ylbenzene-1,3-diol
2-[(1R)-3-Methyl-6-prop-1-en-2-ylcyclohex-2-
en-1-yl]-5-pentylbenzene-1,3-diol; propane
2-[(1S)-3-Methylcyclohex-2-en-1-yl]-4-(3-
methylcyclohex-2-en-1-yl)benzene-1,3,5-triol
5-[(E)-2-(4-Hydroxyphenyl)ethenyl]-4-(3-
methylcyclohex-2-en-1-yl)benzene-1,3-diol
Ethane; 5-methyl-4-(3-methylcyclohex-2-en-
1-yl)benzene-1,3-diol; prop-1-yne
Ethane; 5-methyl-2-(3-methylcyclohex-2-en-
1-yl)benzene-1,3-diol; prop-1-yne
(3R,4R)-1,3-Dimethyl-4-prop-1-en-2-ylcyclohexene;
5-propylbenzene-1,3-diol
2-(6-Ethenyl-3,6-dimethylcyclohex-2-en-1-
yl)-5-pentylbenzene-1,3-diol
Ethane; 5-[1-(3-hydroxyphenyl)propyl]-2-methylphenol
5-Hexyl-2-(3-methyl-6-prop-1-en-2-ylcyclohex-
2-en-1-yl)benzene-1,3-diol
4-[(E)-5-(4-Hydroxyphenyl)hex-2-en-3-yl]-
5-methylbenzene-1,3-diol
5-[1-(3-Hydroxy-4-methylphenyl)propyl]-2-methylphenol
Ethane; 2-(3-methylcyclohex-2-en-1-yl)-5-
pentylbenzene-1,3-diol; 2-methylprop-1-ene
2-[(2E)-3,7-Dimethylocta-2,6-dienyl]-5-
pentylbenzene-1,3-diol; ethane
5-Ethyl-2-[(6R)-3-methyl-6-prop-1-en-2-
ylcyclohex-2-en-1-yl]benzene-1,3-diol
2-[(1R)-3-Methyl-6-prop-1-en-2-ylcyclohex-
2-en-1-yl]-5-propan-2-ylbenzene-1,3-diol
2-[(1R)-3-Methyl-6-prop-1-en-2-ylcyclohex-
2-en-1-yl]benzene-1,3-diol
5-(4-Hydroxy-2,4-dimethylpentan-2-yl)-2-[(1R)-3-methyl-
6-prop-1-en-2-ylcyclohex-2-en-1-yl]benzene-1,3-diol
2-[(1S)-3-Methylcyclohex-2-en-1-yl]-5-
pentylbenzene-1,3-diol; prop-1-ene
2-(3-Methylcyclohex-2-en-1-yl)-5-pentylphenol; prop-1-ene
Ethane; 5-ethylbenzene-1,3-diol
4-Fluoro-5-methyl-2-(3-methylcyclohex-2-
en-1-yl)benzene-1,3-diol; prop-1-ene
2-Fluoroprop-1-ene; 5-methyl-2-(3-methylcyclohex-
2-en-1-yl)benzene-1,3-diol
Ethane; 2-ethyl-5-[(Z)-2-methyloct-3-en-2-yl]benzene-1,3-diol
5-[1-[(Z)-Hex-1-enyl]cyclopentyl]-2-(3-methylcyclohex-
2-en-1-yl)benzene-1,3-diol; propane
2-[(1S,2S)-5-Methylidene-2-prop-1-en-2-ylcyclohexyl]-
5-(2-methyloctan-2-yl)benzene-1,3-diol
2-(3-Methylcyclohex-2-en-1-yl)-5-pentylbenzene-1,3-diol; propane
5-(2-Methylpropyl)-4-propan-2-ylbenzene-1,3-diol
Ethane; 4-propan-2-ylbenzene-1,3-diol; prop-1-en-2-ol
Ethane; 2-(4-methylcyclohex-3-en-1-yl)propan-2-ol;
5-methyl-2-propan-2-ylphenol
Ethane; propane; 2,4,5-trimethylbenzene-1,3-diol
5-(2,6-Dihydroxyphenyl)-2-methylbenzene-1,3-diol; ethane
4-[(3E)-4-(4-Hydroxyphenyl)buta-1,3-dien-2-yl]-5-
methyl-2-(3-methylbut-2-enyl)benzene-1,3-diol
Ethane; 2-[(6R)-3-methyl-6-prop-1-en-2-ylcyclohex-
2-en-1-yl]-5-pentylbenzene-1,3-diol
5-Methylbenzene-1,3-diol; 2-methylpropane
5-Heptyl-2-(3-methylcyclohex-2-en-1-yl)phenol; prop-1-ene
5-[(Z)-Hept-1-enyl]-2-(3-methylcyclohex-2-en-
1-yl)benzene-1,3-diol; prop-1-ene
5-Propyl-4-[(4R,5Z)-2,3,6-trimethylocta-
1,5-dien-4-yl]benzene-1,3-diol
2-[(1S)-3-Methylcyclohex-2-en-1-yl]benzene-1,3,5-triol; prop-1-ene
5-(4-Bromobutyl)-2-[(1S,6S)-3-methyl-6-propan-2-
ylcyclohex-2-en-1-yl]benzene-1,3-diol
5-Heptyl-2-(3-methylcyclohex-2-en-1-yl)-4-[(1R)-3-methyl-
6-prop-1-en-2-ylcyclohex-2-en-1-yl]benzene-1,3-diol; prop-1-ene
5-Fluoro-2-(3-methylcyclohex-2-en-1-
yl)benzene-1,3-diol; prop-1-ene
4-[(1R)-3-Methyl-6-prop-1-en-2-ylcyclohex-
2-en-1-yl]-5-propylbenzene-1,3-diol
5-(Bromomethyl)-2-[(1R,6R)-6-tert-butyl-3-
methylcyclohex-2-en-1-yl]benzene-1,3-diol
5-(Bromomethyl)-2-[(1S)-3-methylcyclohex-
2-en-1-yl]benzene-1,3-diol
4-[(2R)-3,4-Dimethylpent-4-en-2-yl]-5-pentylbenzene-1,3-diol
5-[(Z)-Hept-1-enyl]-4-[(1R)-3-methyl-6-prop-1-
en-2-ylcyclohex-2-en-1-yl]benzene-1,3-diol
5-Heptyl-2-(3-methylcyclohex-2-en-1-yl)benzene-1,3-diol; prop-1-ene
Ethane; 5-methyl-2-(3-methylcyclohex-2-en-1-
yl)benzene-1,3-diol; prop-1-ene
5-(Bromomethyl)-2-(3-methylcyclohex-2-en-1-
yl)benzene-1,3-diol; prop-1-ene
5-Methyl-2-(3-methylcyclohex-2-en-1-yl)benzene-1,3-diol; prop-1-ene
2-(3-Methylcyclohex-2-en-1-yl)-5-propylbenzene-1,3-diol; prop-1-ene
5-Propyl-4-[(4R)-2,3,6-trimethylhepta-1,5-dien-4-yl]benzene-1,3-diol
2-[3,5-Dihydroxy-4-(3-methylcyclohex-2-en-
1-yl)phenyl]acetonitrile; prop-1-ene
But-2-yne; 4-(3-methylcyclohex-2-en-1-yl)-5-pentylbenzene-1,3-diol
5-Fluoro-4-[(1R)-3-methyl-6-prop-1-en-2-
ylcyclohex-2-en-1-yl]benzene-1,3-diol
5-Heptyl-4-[(1R)-3-methyl-6-prop-1-en-2-
ylcyclohex-2-en-1-yl]benzene-1,3-diol
4-[(4S,5Z)-2-Hydroxy-6-methylocta-1,5-
dien-4-yl]-5-methylbenzene-1,3-diol
5-Ethylbenzene-1,3-diol; hexane
5-[(2Z,4E,5Z)-4-Ethylidene-3-(2-methyl-1-
phenylbutylidene)boranylhepta-2,5-dien-2-yl]benzene-1,3-diol
Ethane; 5-[(E)-2-(4-methylphenyl)ethenyl]benzene-1,3-diol
Ethane; 5-(2-methyloctan-2-yl)benzene-1,3-diol
Ethane; 5-[4-(2-hydroxyethyl)phenyl]benzene-1,3-diol
Methane; 2-[(1S)-3-methylcyclohex-2-en-1-yl]-
5-pentylbenzene-1,3-diol; prop-1-ene
Ethane; 2-(3-methyl-6-prop-1-en-2-ylcyclohex-
2-en-1-yl)-5-pentylbenzene-1,3-diol
2-Methylbut-2-ene; 4-methylphenol; propane;
4-propan-2-ylbenzene-1,3-diol
4-Propan-2-yl-6-prop-1-en-2-ylbenzene-1,3-diol
4-[(E)-But-2-en-2-yl]-6-propan-2-ylbenzene-1,3-diol
2-(2-Ethyl-5-methylcyclohexyl)-5-pentylbenzene-1,3-diol
2-(12-Hydroxydodecyl)-5-[(E)-2-phenylethenyl]benzene-1,3-diol
4-Butyl-2,5-dimethylbenzene-1,3-diol
5-Methyl-2-[(1S)-3-methylcyclohex-2-en-1-yl]benzene-1,3-diol
5-Methyl-2-[(1S)-3-methylcyclohex-2-en-1-
yl]benzene-1,3-diol; prop-1-ene
2-(3-Methylcyclohex-2-en-1-yl)-5-pentylbenzene-
1,3-diol; prop-1-ene
Ethane; 2-[(1S,6R)-6-ethyl-3-methylcyclohex-2-
en-1-yl]-3-methoxy-5-pentylphenol
2-[(1S,6R)-3,6-Dimethylcyclohex-2-en-1-yl]-5-
pentylbenzene-1,3-diol; prop-1-ene
2-Methyl-5-[(E)-prop-1-enyl]benzene-1,3-diol
4-Heptan-4-ylbenzene-1,3-diol
2-Methyl-5-(3,7,7-trimethyloctan-3-yl)benzene-1,3-diol
2-Ethyl-5-(2,6,6-trimethylheptan-2-yl)benzene-1,3-diol
5-(6,6-Dimethylhept-4-yn-2-yl)-2-methylbenzene-1,3-diol
2-Butylbenzene-1,3-diol; ethane; 2-methylhexane
2-[(3,5-Dihydroxyphenyl)methyl]-3,5,6-trimethylbenzene-1,4-diol
2-[6-[(E)-But-2-en-2-yl]-3-methylcyclohex-
2-en-1-yl]-5-pentylphenol; ethane
5-Butyl-2-(6-ethenyl-3,6-dimethylcyclohex-2-en-1-yl)benzene-1,3-diol
(3-Hydroxy-5-propan-2-ylphenyl)oxidanium
Ethane; 2-[(1R,6R)-3-methyl-6-prop-1-en-2-
ylcyclohex-2-en-1-yl]-5-pentylbenzene-1,3-diol
4-(1-Phenylhexyl)benzene-1,3-diol
4-(2-Phenylbutyl)benzene-1,3-diol
4-(3-Phenylbutyl)benzene-1,3-diol
4-(2-Phenylhexyl)benzene-1,3-diol
4-(1-Phenylbutyl)benzene-1,3-diol
2-[(4-Ethylcyclohexyl)-(2-hydroxyphenyl)methyl]-5-methylphenol
2-(3,6-Dimethylcyclohex-2-en-1-yl)-5-
pentylbenzene-1,3-diol; prop-1-ene
5-(2-Methyloctan-2-yl)-2-[(1S,2S)-4,6,6-trimethyl-
2-bicyclo[3.1.1]heptanyl]benzene-1,3-diol
2-[(2R)-Butan-2-yl]-5-(3-methylpentan-3-yl)benzene-1,3-diol
Ethane; 5-(2-methylhex-5-en-2-yl)benzene-1,3-diol
Ethane; 5-(2-methylhexan-2-yl)benzene-1,3-diol
2-(2-But-1-en-2-yl-5-methylphenyl)-5-methylbenzene-1,3-diol
(Z)-But-2-ene; ethane; 2-(3-methylphenyl)-5-pentylbenzene-1,3-diol
2-(3-Methylphenyl)-5-pentylbenzene-1,3-diol; prop-1-ene
5-(6,6-Dimethylheptan-2-yl)-2-methylbenzene-1,3-diol; ethane
Methylcyclopentane; 5-propylbenzene-1,3-diol
Ethane; 2-methyl-5-(trifluoromethyl)benzene-1,3-diol
2-(2-Hydroxy-4-methylphenyl)-5-methylbenzene-1,3-diol
Ethane; 4,5,6-trimethyl-2-[(6R)-3-methyl-6-prop-1-
en-2-ylcyclohex-2-en-1-yl]benzene-1,3-diol
5-[(E)-6-Bromo-2-methylhept-5-en-2-yl]-2-
methylbenzene-1,3-diol; ethane
5-(2,6-Dimethylheptan-2-yl)benzene-1,3-diol; ethane
[2-[(E)-2-[4-[(2E)-3,7-Dimethylocta-2,6-dienyl]-3,5-
dihydroxyphenyl]ethenyl]-5-hydroxyphenyl]oxidanium
5-[(1E,3Z,5E)-6-Hydroxy-3-(hydroxymethyl)octa-
1,3,5-trienyl]-2-(3-methylbut-2-enyl)benzene-1,3-diol
5-Methyl-2-[(6R)-3-methyl-6-prop-1-en-2-ylcyclohex-
3-en-1-yl]benzene-1,3-diol
2,5-Dimethylbenzene-1,3-diol; ethane
2,4,5-Tripentylbenzene-1,3-diol
2-[1-(2,4,6-Trihydroxyphenyl)butyl]benzene-1,3,5-triol
2-[1-(2,4,6-Trihydroxyphenyl)pentyl]benzene-1,3,5-triol
4-(1-Phenylethyl)benzene-1,3-diol; hydrate
5-Ethenyl-2-propan-2-ylbenzene-1,3-diol
5-(Bromomethyl)-2-propan-2-ylbenzene-1,3-diol
2-Butyl-5-(2-phenylethyl)benzene-1,3-diol
4-(4-Hydroxy-2-methylphenyl)-2,5,6-trimethylbenzene-1,3-diol
4-Hexan-3-ylbenzene-1,3-diol
2-(2-Methylpropyl)-5-(4-phenylbuta-1,3-dienyl)benzene-1,3-diol
4-Cyclododecylbenzene-1,3-diol
2-Propan-2-yl-5-prop-2-enylbenzene-1,3-diol
4-Cyclohexa-2,4-dien-1-ylbenzene-1,3-diol
5-Methyl-2-pentylbenzene-1,3-diol
2-(1,6-Dimethylcyclohexa-2,4-dien-1-yl)benzene-1,3-diol
4-[(2E)-3,7-Dimethylocta-2,6-dienyl]-2,5-dimethylbenzene-1,3-diol
2-[(E)-5-(4-Hydroxyphenyl)pent-4-en-2-yl]benzene-1,3-diol
2-[(E)-12-Ethyl-14-phenyltetradec-13-enyl]benzene-1,3-diol
2-(5-Methyl-2-propan-2-ylcyclohexyl)benzene-1,3-diol
5-(3-Phenylprop-1-en-2-yl)-2-propan-2-ylbenzene-1,3-diol
2-(3-Propan-2-ylphenyl)benzene-1,3-diol
5-(2-Propan-2-ylphenyl)benzene-1,3-diol
5-[1-(3,5-Dihydroxy-4-methylphenyl)tetradecyl]-
2-methylbenzene-1,3-diol
5-Dec-1-enylbenzene-1,3-diol
5-Hexyl-2-[(1R,6R)-3-methyl-6-prop-1-en-2-
ylcyclohex-2-en-1-yl]benzene-1,3-diol
4,6-Diiodo-2-[(1R,6R)-3-methyl-6-prop-1-en-2-ylcyclohex-
2-en-1-yl]-5-pentylbenzene-1,3-diol
5-Butyl-2-cyclohexylphenol
2-Propan-2-ylbenzene-1,3-diol; hydrochloride
5-[(E)-2-(4-Hydroxyphenyl)ethenyl]-2,4,6-trimethylbenzene-1,3-diol
2-[(2E)-3,7-Dimethylocta-2,6-dienyl]-5-[2-
(4-hydroxyphenyl)ethenyl]benzene-1,3-diol
2-(Cyclohexylmethyl)-5-methylbenzene-1,3-diol
5-Methyl-2-undecylbenzene-1,3-diol
2-Hexadecyl-5-methylbenzene-1,3-diol
5-Methyl-2-pentadecylbenzene-1,3-diol
4-Ethyl-2-hexyl-5-methylbenzene-1,3-diol
5-[(E)-2-Phenylethenyl]-4-propan-2-ylbenzene-1,3-diol
2,4-Dimethyl-5-pentylbenzene-1,3-diol
2-[(Z)-1-[(E)-But-1-enyl]peroxybut-1-enyl]-5-propan-2-ylphenol
2-(7-Methyloctyl)-3-methylperoxyphenol
2-[(2E)-3,7-Dimethylocta-2,6-dienyl]-4,5-dipropylbenzene-1,3-diol
5-Benzyl-2-cyclohexylbenzene-1,3-diol
2-Cyclohexyl-5-methyl-4-phenylbenzene-1,3-diol
2-Cyclohexyl-4,5-dimethylbenzene-1,3-diol
5-Benzyl-2-phenylbenzene-1,3-diol
2,5-Dibenzylbenzene-1,3-diol
2-Benzyl-4-cyclohexyl-5-methylbenzene-1,3-diol
2-(1-Adamantyl)-5-methylbenzene-1,3-diol
2-(1-Adamantyl)-5-tert-butylbenzene-1,3-diol
2-(1-Adamantyl)-5-(2-methylbutan-2-yl)benzene-1,3-diol
2-Pentan-3-ylbenzene-1,3-diol
2-Cyclohex-2-en-1-yl-5-(3-ethylnonan-3-yl)benzene-1,3-diol
5-[4-[4-(4-Pentylcyclohexyl)
cyclohexyl]phenyl]methyl]benzene-1,3-diol
4-[1-(2,4-Dihydroxyphenyl)-2-methylpropyl]benzene-1,3-diol
5-[Bis(2-hydroxy-3,4,6-trimethylphenyl)methyl]benzene-1,3-diol
5-Prop-1-en-2-ylbenzene-1,3-diol
2,4,5-Tributylbenzene-1,3-diol
2,4-Dimethyl-5-propan-2-ylbenzene-1,3-diol
2-[1-(2-Hydroxy-4-propylphenyl)butyl]-5-propylphenol
2-[2-(2-Hydroxy-4-methylphenyl)-4-
methylpentan-2-yl]-5-methylphenol
5-Methyl-2-(2,6,10-trimethyldodecan-4-yl)benzene-1,3-diol
5-Methyl-2-[(2E,6E)-3,7,11-trimethyl-4-(3-methylbut-
2-enyl)dodeca-2,6,10-trienyl]benzene-1,3-diol
5-Methyl-2-(2,6,10-trimethyldodecan-2-yl)benzene-1,3-diol
2-[(2E,4E)-5,9-Dimethyl-2-(6-methylhept-5-en-2-
ylidene)deca-4,8-dienyl]-5-methylbenzene-1,3-diol
5-Methyl-4-(2,6,10-trimethyldodecyl)benzene-1,3-diol
2,4-Ditert-butyl-5-propan-2-ylbenzene-1,3-diol
2-[(2,6-Dihydroxy-4-propylphenyl)methyl]-5-propylbenzene-1,3-diol
2-[(2,4-Dihydroxy-6-propylphenyl)methyl]-5-propylbenzene-1,3-diol
2-(2-Phenylpropyl)benzene-1,3-diol
2-(2-Hydroxyphenyl)-5-phenylbenzene-1,3-diol
2-[(1R,6R)-3-(Hydroxymethyl)-6-prop-1-en-2-
ylcyclohex-3-en-1-yl]-5-propylbenzene-1,3-diol
2-[(1S,6S)-3-(Hydroxymethyl)-6-prop-1-en-2-
ylcyclohex-3-en-1-yl]-5-propylbenzene-1,3-diol
2-[(1R,6R)-3-Methyl-6-prop-1-en-2-ylcyclohex-
3-en-1-yl]-5-propylbenzene-1,3-diol
2-[(1S,6S)-3-(Hydroxymethyl)-6-prop-1-en-2-
ylcyclohex-2-en-1-yl]-5-propylbenzene-1,3-diol
2-[(1S,6S)-3-Methyl-6-prop-1-en-2-ylcyclohex-
3-en-1-yl]-5-propylbenzene-1,3-diol
2-[(1R,6S)-3-Methyl-6-prop-1-en-2-ylcyclohexa-
2,4-dien-1-yl]-5-propylbenzene-1,3-diol
2-[(2R,3S)-5-Methyl-2-prop-1-en-2-yl-2,3,6,7-
tetrahydrothiepin-3-yl]-5-pentylbenzene-1,3-diol
2-[(1R,5R)-3-Methyl-5-prop-1-en-2-ylcyclopent-
2-en-1-yl]-5-pentylbenzene-1,3-diol
5-(1-lodopropan-2-yl)benzene-1,3-diol
5-[(E)-3-Methyloct-2-en-2-yl]-2-(3-methyl-6-prop-
1-en-2-ylcyclohex-2-en-1-yl)phenol
2-(3-Methyl-6-prop-1-en-2-ylcyclohex-2-
en-1-yl)-5-octan-2-ylphenol
5-(2-Methylheptyl)-2-(3-methyl-6-prop-1-en-2-
ylcyclohex-2-en-1-yl)phenol
Ethanol; 2-[(1R,6R)-3-methyl-6-prop-1-en-2-ylcyclohex-
2-en-1-yl]-5-pentylbenzene-1,3-diol
[5-[3,5-Dihydroxy-4-[(1R)-3-methyl-6-prop-1-en-2-
ylcyclohex-2-en-1-yl]phenyl]-5-methylhexyl] nitrate
5-Methyl-2-[(E)-3-methylpent-2-enyl]benzene-1,3-diol
2-[(1S,6S)-6-Ethenyl-3-methylcyclohex-2-en-1-yl]-
3-methoxy-5-pentylphenol
5-[3-(2-Hydroxyphenyl)propyl]benzene-1,3-diol
2-(10-Hydroxydecan-5-yl)-5-undecylbenzene-1,3-diol
5-[2-(3-Hydroxy-4-propan-2-ylphenyl) ethyl]-2-propan-2-ylphenol
4-(3,5-Ditert-butyl-4-hydroxyphenyl)-2,5-dimethylbenzene-1,3-diol
2-Tert-butyl-5-pentan-2-ylbenzene-1,3-diol
2-(5-Methyl-2-prop-1-en-2-ylcyclohex-2-en-1-
yl)-5-pentylbenzene-1,3-diol
2-(3-Methyl-6-prop-1-en-2-ylcyclohexen-
1-yl)-5-pentylbenzene-1,3-diol
5-[2-(2-Hydroxycyclobut-2-en-1-yl)ethyl]benzene-1,3-diol
2-[(1S,6S)-3-Methyl-6-(2-methylprop-2-enyl)cyclohex-
2-en-1-yl]-5-pentylbenzene-1,3-diol
2-[(6S)-4-Fluoro-3-methyl-6-propan-2-ylcyclohex-2-
en-1-yl]-5-pentylbenzene-1,3-diol
2-[3-(Fluoromethyl)cyclohex-2-en-1-yl]-5-(2-
methyloctan-2-yl)benzene-1,3-diol
2-[(6S)-3-(Hydroxymethyl)-6-propan-2-ylcyclohex-
2-en-1-yl]-5-pentylbenzene-1,3-diol
2-[(6S)-4-Hydroxy-3-methyl-6-propan-2-ylcyclohex-
2-en-1-yl]-5-pentylbenzene-1,3-diol
2-[(6S)-4-Fluoro-3-methyl-6-propan-2-ylcyclohex-
2-en-1-yl]-5-methylbenzene-1,3-diol
2-[(2E)-3,7-Dimethylocta-2,6-dienyl]-5-[(Z)-
2-phenylethenyl]benzene-1,3-diol
2-(5-Methylidene-2-prop-1-en-2-ylcyclohexyl)-
5-pentylbenzene-1,3-diol
[5-[3,5-Dihydroxy-4-[(1S)-3-methylcyclohex-2-en-1-
yl]phenyl]-5-methylhexyl] nitrate
[5-[3,5-Dihydroxy-4-[(1R,6R)-3-methyl-6-prop-1-en-
2-ylcyclohex-3-en-1-yl]phenyl]-5-methylhexyl] nitrate
2-Methyl-5-pentadeca-1,11-dienylbenzene-1,3-diol
2-Methyl-5-[(E)-pentadec-1-enyl]benzene-1,3-diol
2-Methyl-5-pentadeca-1,3-dienylbenzene-1,3-diol
2-Methyl-5-pentadeca-1,3,5-trienylbenzene-1,3-diol
4-(3-Methylbutan-2-yl)benzene-1,3-diol
2-[(1S,6S)-6-Ethenyl-3-methylcyclohex-2-en-1-
yl]-5-pentylbenzene-1,3-diol
3-(4-Decyl-2,6-dihydroxyphenyl)-1-phenylprop-2-ene-1-thione
2-[(1R,6R)-3-Methyl-6-prop-1-en-2-ylcyclohex-2-en-1-
yl]-5-pentylbenzene-1,3-diol; 5-pentylbenzene-1,3-diol
Benzene-1,4-diol; 2-[(1R,6R)-3-methyl-6-prop-1-en-2-
ylcyclohex-2-en-1-yl]-5-pentylbenzene-1,3-diol
2,5-Dioctylbenzene-1,3-diol
5-(6-Methyloctyl)-2-[(1R,6R)-3-methyl-6-prop-1-
en-2-ylcyclohex-2-en-1-yl]benzene-1,3-diol
5-Hex-5-enyl-2-[(1R,6R)-3-methyl-6-prop-1-en-
2-ylcyclohex-2-en-1-yl]benzene-1,3-diol
5-(4-Pentylcyclohexyl)benzene-1,3-diol
5-[4-(4-Pentylcyclohexyl)phenyl]benzene-1,3-diol
2-Cyclopentyl-5-ethylbenzene-1,3-diol
5-Pentyl-2-propan-2-ylbenzene-1,3-diol
2-Cyclopentyl-5-pentylbenzene-1,3-diol
5-[(2S)-2-Methylundecyl]benzene-1,3-diol
2-[(6Z)-1-Hydroxy-3,7,11-trimethyldodeca-2,6,10-
trienyl]-5-methylbenzene-1,3-diol
5-(1-Hexylcyclopentyl)-2-[(6R)-3-methyl-6-prop-1-
en-2-ylcyclohex-2-en-1-yl]benzene-1,3-diol
2-[(1R)-3-Methyl-6-(2-trimethylsilyloxypropan-2-
yl)cyclohex-3-en-1-yl]-5-pentyl-3-tri(propan-2-yl)silyloxyphenol
2-[(1R,6R)-3-Methyl-6-(2-trimethylsilyloxypropan-2-
yl)cyclohex-3-en-1-yl]-5-pentyl-3-tri(propan-2-yl)silyloxyphenol
4-[(1R,2S,5R)-5-Hydroxy-5-methyl-2-(6-methylhepta-
1,5-dien-2-yl)cyclohexyl]-5-methylbenzene-1,3-diol
2-[(1R,6R)-6-Tert-butyl-3-methylcyclohex-2-en-1-
yl]-5-pentylbenzene-1,3-diol
5-Ethenyl-2-[(E)-3-methylbut-1-enyl]benzene-1,3-diol
5-Pentyl-2-(2-prop-1-en-2-ylcyclohexen-1-yl)benzene-1,3-diol
2-[(1R,2R)-5-Methylidene-2-prop-1-en-2-ylcyclohexyl]-
5-pentylbenzene-1,3-diol
4-(Hydroxymethyl)-2-(3-methyl-6-prop-1-en-2-ylcyclohex-
2-en-1-yl)-5-pentylbenzene-1,3-diol
2-(4-Methylpentan-2-yl)-5-pentylbenzene-1,3-diol
4-(2,5-Dimethylhexan-3-yl)benzene-1,3-diol
4-[(1R,6R)-3-Methyl-6-prop-1-en-2-ylcyclohex-2-en-1-yl]-2-
[(1S,6S)-3-methyl-6-prop-1-en-2-ylcyclohex-2-en-1-yl]benzene-1,3,5-
triol
5-[(E)-2-(4-Hydroxyphenyl)ethenyl]-4-[(1S,6S)-3-methyl-
6-prop-1-en-2-ylcyclohex-2-en-1-yl]benzene-1,3-diol
2,4-Bis[(2E)-3,7-dimethylocta-2,6-dienyl]-5-methylbenzene-1,3-diol
4-[(2E)-3,7-Dimethylocta-2,6-dienyl]-5-methylbenzene-1,3-diol
5-Fluoro-2-[(6R)-3-methyl-6-prop-1-en-2-ylcyclohex-
2-en-1-yl]benzene-1,3-diol
5-[(E)-2-(4-Hydroxyphenyl)ethenyl]-2-[(1R,6R)-3-methyl-
6-prop-1-en-2-ylcyclohex-2-en-1-yl]benzene-1,3-diol
2-[(1R,6R)-3-Methyl-6-prop-1-en-2-ylcyclohex-2-en-1-yl]-4-
[(6S)-3-methyl-6-prop-1-en-2-ylcyclohex-
2-en-1-yl]benzene-1,3,5-triol
5-[(E)-2-(4-Methylphenyl)ethenyl]-2-(3-methyl-6-prop-
1-en-2-ylcyclohex-2-en-1-yl)benzene-1,3-diol
5-[(E)-2-(4-Methylphenyl)ethenyl]-4-(3-methyl-6-prop-
1-en-2-ylcyclohex-2-en-1-yl)benzene-1,3-diol
4-[1-(2,4-Dihydroxyphenyl)-3-methylbutyl]benzene-1,3-diol
4-[(1R,6S)-3-Methyl-6-propan-2-ylcyclohex-2-en-1-
yl]-5-[(E)-2-phenylethenyl]benzene-1,3-diol
2-Methyl-5-pentacosylbenzene-1,3-diol
5-Heptadecyl-2-methylbenzene-1,3-diol
5-Heptacosyl-2-methylbenzene-1,3-diol
5-[(E)-Dec-1-enyl]benzene-1,3-diol
2-(2-Hydroxy-6-methylhept-5-en-2-yl)-5-methylphenol
2-(5-Methyl-2-prop-1-en-2-
ylcyclohexen-1-yl)-5-pentylbenzene-1,3-diol
2-(5-Methyl-2-prop-1-en-2-ylcyclohex-3-
en-1-yl)-5-pentylbenzene-1,3-diol
2-[6-(1-Fluoroethenyl)-3-methylcyclohex-
2-en-1-yl]-5-methylbenzene-1,3-diol
4-Fluoro-5-methyl-2-(3-methyl-6-prop-1-en-
2-ylcyclohex-2-en-1-yl)benzene-1,3-diol
5-(1-Phenylethyl)benzene-1,3-diol
5-(2-Ethylhexyl)benzene-1,3-diol
2-(1-Phenylpropyl)benzene-1,3,5-triol
2-[(2E)-3,7-Dimethylocta-2,6-dienyl]benzene-1,3-diol
5-Butyl-2-[(2E)-3,7-dimethylocta-2,6-dienyl]benzene-1,3-diol
2-[(2E)-3,7-Dimethylocta-2,6-dienyl]-4-
methyl-5-propylbenzene-1,3-diol
4-[1-(4-Tert-butylphenyl)ethyl]benzene-1,3-diol
2-(Cyclohexylmethyl)benzene-1,3-diol
2-[(6S)-3-Methyl-6-propan-2-ylcyclohex-3-
en-1-yl]-5-propylbenzene-1,3-diol
5-[(9Z,11E)-9-[(E)-1-Hydroxyprop-1-enyl]-7,10-
dimethylpentadeca-9,11,14-trien-6-yl]-2-methylphenol
2-[(E)-1-(2-Hydroxyphenyl)but-1-enyl]-5-propan-2-ylphenol
5-[(E)-3-Phenylbut-2-en-2-yl]benzene-1,3-diol
3-Methoxy-2-[(6R)-3-methyl-6-prop-1-en-2-
ylcyclohex-2-en-1-yl]-5-pentylphenol
5-(8-Bromo-2-methyloctan-2-yl)-2-[(1R,6S)-3-(hydroxymethyl)-
6-propan-2-ylcyclohex-3-en-1-yl]benzene-1,3-diol
5-(5-Bromopentyl)-2-[(1S,6S)-3-methyl-6-propan-
2-ylcyclohex-2-en-1-yl]benzene-1,3-diol
2-But-1-en-2-yl-5-pentylbenzene-1,3-diol
2-[(2E)-3-(Hydroxymethyl)-7-methylocta-
2,7-dienyl]-5-pentylbenzene-1,3-diol
5-Pentyl-2-(2,3,6-trimethylhepta-1,5-dien-4-yl)benzene-1,3-diol
5-Benzyl-2-methylbenzene-1,3-diol
2-Cyclohex-2-en-1-ylbenzene-1,3-diol
5-Heptadeca-7,12-dienylbenzene-1,3-diol
5-Methyl-4-(3-methylcyclohexen-1-yl)benzene-1,3-diol
5-Heptadec-2-enylbenzene-1,3-diol
4-Deuterio-2-(1-phenylethyl)benzene-1,3-diol
2,4-Dideuterio-5-(1,1,2,2,3,3,4-
heptadeuteriopentyl)benzene-1,3-diol
5-Decyl-2-nonylbenzene-1,3-diol
5-Methyl-4-pent-3-en-2-ylbenzene-1,3-diol
5-Methyl-2-pent-3-en-2-ylbenzene-1,3-diol
2-[2-(4-Methylphenyl)propyl]-5-pentylbenzene-1,3-diol
5-(2-Methyloctan-2-yl)-2-(2,6,6-trimethyl-1-
bicyclo[3.1.1]heptanyl)benzene-1,3-diol
5-[2-(4-Hydroxyphenyl)ethenyl]-4-(3-
methylbut-1-enyl)benzene-1,3-diol
5-[(E)-2-(2,4-Dihydroxyphenyl)ethenyl]-
2-(3-methylbut-3-enyl)benzene-1,3-diol
2-Dodecan-2-yl-5-methylbenzene-1,3-diol
5-[2-(4-Hydroxyphenyl)ethenyl]-2-(3-
methylbut-3-enyl)benzene-1,3-diol
5-Methyl-2-(2,6,10-trimethyldodecyl)benzene-1,3-diol
2-(3-Methylbut-2-enyl)-5-(3-phenylprop-2-enyl)benzene-1,3-diol
5-Heptyl-2-hexylbenzene-1,3-diol
Undecenylresorcin
5-Pentyl-2-[(2E)-2,3,7-trimethylocta-2,6-dienyl]benzene-1,3-diol
5-Propyl-2-[(2E)-2,3,7-trimethylocta-2,6-dienyl]benzene-1,3-diol
5-Ethyl-2-[(1R,6R)-3-methyl-6-prop-1-en-
2-ylcyclohex-2-en-1-yl]benzene-1,3-diol
5-Butyl-2-(3-methyl-6-prop-1-en-2-ylcyclohex-2-en-1-yl)phenol
2-Octan-3-ylbenzene-1,3,5-triol
4-[Bis(6-ethyl-4-hydroxy-2,3-dimethylcyclohexa-
2,4-dien-1-yl)methyl]benzene-1,3-diol
4-[(3E,5E,7Z)-6-Hydroxy-3,5,7-trimethylnona-
3,5,7-trien-2-yl]benzene-1,3-diol
4-(1-Cyclohexa-1,5-dien-1-ylethyl)benzene-1,3-diol
CID 127021089
2-[(5S)-10-Hydroxydecan-5-yl]-5-(2-methylundecyl)benzene-1,3-diol
5-[1-(4-Hydroxyphenyl)propan-2-yl]benzene-1,3-diol
1-[(1R,2R)-2-(2,6-Dihydroxy-4-pentylphenyl)-
4-methylcyclohex-3-en-1-yllethenyl hypoiodite
5-[(4Z,5Z)-4-Ethylidene-7-hydroxyocta-5,7-dienyl]benzene-1,3-diol
5-Methyl-2-[(1S,6S)-3-methyl-6-prop-1-en-2-
ylcyclohex-2-en-1-yl]benzene-1,3-diol
2-(3-Ethyl-6-prop-1-en-2-ylcyclohex-2-en-1-yl)-
5-pentylbenzene-1,3-diol
2-[(3R,4Z)-2,5-Dimethylocta-1,4-dien-3-yl]-5-pentylbenzene-1,3-diol
2-[(1S)-3-(Hydroxymethyl)cyclohex-2-en-1-yl]-
5-pentylbenzene-1,3-diol
2-(4-Fluoro-3-methylcyclohex-2-en-1-yl)-5-
(2-methyloctan-2-yl)benzene-1,3-diol
4-Fluoro-5-methyl-2-(3-methylcyclohex-2-en-1-yl)benzene-1,3-diol
2-(4-Fluoro-3-methylcyclohex-2-en-1-yl)-5-pentylbenzene-1,3-diol
2-(4-Hydroxy-3-methylcyclohex-2-en-1-yl)-5-pentylbenzene-1,3-diol
2-[(6S)-3-(Fluoromethyl)-6-propan-2-ylcyclohex-
2-en-1-yl]-5-pentylbenzene-1,3-diol
2-[3-(Hydroxymethyl)cyclohex-2-en-1-yl]-5-pentylbenzene-1,3-diol
2-[3-(Fluoromethyl)cyclohex-2-en-1-yl]-5-pentylbenzene-1,3-diol
2-[(1R)-3-(Fluoromethyl)-6-propan-2-ylcyclohex-
2-en-1-yl]-5-(2-methyloctan-2-yl)benzene-1,3-diol
2-(4-Hydroxy-3-methyl-6-propan-2-ylcyclohex-2-en-
1-yl)-5-(2-methyloctan-2-yl)benzene-1,3-diol
2-(4-Fluoro-3-methyl-6-propan-2-ylcyclohex-2-en-
1-yl)-5-(2-methyloctan-2-yl)benzene-1,3-diol
2-[(6S)-4-Fluoro-3-methyl-6-propan-2-ylcyclohex-2-
en-1-yl]-5-(2-methyloctan-2-yl)benzene-1,3-diol
2-[(6S)-4-Hydroxy-3-methyl-6-propan-2-ylcyclohex-
2-en-1-yl]-5-(2-methyloctan-2-yl)benzene-1,3-diol
2-[(6S)-3-(Hydroxymethyl)-6-propan-2-ylcyclohex-
2-en-1-yl]-5-(2-methyloctan-2-yl)benzene-1,3-diol
2-[3-(Fluoromethyl)-6-propan-2-ylcyclohex-2-
en-1-yl]-5-(2-methyloctan-2-yl)benzene-1,3-diol
2-[(6S)-3-(Fluoromethyl)-6-propan-2-ylcyclohex-2-
en-1-yl]-5-(2-methyloctan-2-yl)benzene-1,3-diol
2-[(1R)-3-Methyl-6-prop-1-en-2-ylcyclohex-2-
en-1-yl]-5-propylbenzene-1,3-diol
5-Hexan-2-yl-4,6-dimethyl-2-(3-methyl-6-prop-1-en-
2-ylcyclohex-2-en-1-yl)benzene-1,3-diol
4,6-Dimethyl-5-(3-methylhexan-2-yl)-2-(3-methyl-6-
prop-1-en-2-ylcyclohex-2-en-1-yl)benzene-1,3-diol
2-[(1S,6S)-3-Methyl-6-prop-1-en-2-ylcyclohex-
3-en-1-yl]-5-pentylbenzene-1,3-diol
4,6-Dimethyl-2-(3-methyl-6-prop-1-en-2-ylcyclohex-
2-en-1-yl)-5-pentylbenzene-1,3-diol
5-[2-(4-Methylphenyl)ethyl]benzene-1,3-diol
2-[(1R,2S,5S)-5-Methyl-2-prop-1-en-2-ylcyclohexyl]-
5-propylbenzene-1,3-diol
5-(1-Hex-1-enylcyclopentyl)-2-[(1R,2R,5S)-4,6,6-trimethyl-
2-bicyclo[3.1.1]hept-3-enyl]benzene-1,3-diol
2-(1-Hydroxypentyl)-5-methylbenzene-1,3-diol
2-(6-Tert-butyl-3-methylcyclohex-2-en-1-yl)-5-
fluorobenzene-1,3-diol
2-(1,4-Dimethyl-2-tricyclo[3.2.0.02,7]hept-3-enyl)-
5-pentylbenzene-1,3-diol
4-[4-(4-Hydroxyphenyl)buta-1,3-dien-2-yl]-5-methyl-
2-(3-methylbut-2-enyl)benzene-1,3-diol
5-[2-(4-Hydroxyphenyl)ethenyl]-2-[1-(4-
hydroxyphenyl)ethyl]benzene-1,3-diol
5-(Bromomethyl)-2-(3-methylcyclohex-2-en-1-yl)benzene-1,3-diol
5-(1-Hex-1-enylcyclopentyl)-2-[3-(hydroxymethyl)-6-
prop-1-en-2-ylcyclohex-2-en-1-yl]benzene-1,3-diol
5-(2,4-Dimethylpentan-2-yl)benzene-1,3-diol
5-(2,2-Dimethylpropyl)-4-propan-2-ylbenzene-1,3-diol
2-[3-Methyl-6-(2-methylprop-2-enyl)cyclohex-2-
en-1-yl]-5-pentylbenzene-1,3-diol
2-[3-Methyl-4-(2-methylpropyl)-6-prop-1-en-2-
ylcyclohex-2-en-1-yl]-5-pentylbenzene-1,3-diol
4-Ethyl-5-methyl-2-(3-methyl-6-prop-1-en-2-
ylcyclohex-2-en-1-yl)benzene-1,3-diol
CID 123730698
5-(1-Hex-1-enylcyclopentyl)-2-[(1S,6S)-3-(hydroxymethyl)-
6-prop-1-en-2-ylcyclohex-2-en-1-yl]benzene-1,3-diol
4-But-2-en-2-yl-6-propan-2-ylbenzene-1,3-diol
5-(1-Hex-1-enylcyclopentyl)-2-[(2R,5S)-4,6,6-trimethyl-
2-bicyclo[3.1.1]hept-3-enyl]benzene-1,3-diol
5-(6-Bromo-2-methylhexan-2-yl)-2-(3-ethyl-6-prop-
1-en-2-ylcyclohex-2-en-1-yl)benzene-1,3-diol
5-(6-Bromo-2-methylhexan-2-yl)-2-(6-prop-1-en-2-
yl-3-propylcyclohex-2-en-1-yl)benzene-1,3-diol
5-(1-Hex-1-enylcyclopentyl)-2-[(1S,2S,5R)-4,6,6-
trimethyl-2-bicyclo[3.1.1]hept-3-enyl]benzene-1,3-diol
3-(3-Methyl-6-prop-1-en-2-ylcyclohex-2-en-1-yl)-
6-[(E)-pent-1-enyl]benzene-1,2,4-triol
5-(Bromomethyl)-2-(6-tert-butyl-3-methylcyclohex-
2-en-1-yl)benzene-1,3-diol
5-Hept-1-enyl-2-(3-methylcyclohex-2-en-1-yl)benzene-1,3-diol
5-Hept-1-enyl-3-methoxy-2-(3-methylcyclohex-2-en-1-yl)phenol
5-[1-(5-Bromopentyl)cyclobutyl]-2-(6-prop-1-en-2-
yl-3-propylcyclohex-2-en-1-yl)benzene-1,3-diol
2-(5-Methylidene-2-prop-1-en-2-ylcyclohexyl)-
5-(2-methyloctan-2-yl)benzene-1,3-diol
3-[(1S,6S)-3-Methyl-6-propan-2-ylcyclohex-2-en-
1-yl]-6-[(E)-pent-1-enyl]benzene-1,2,4-triol
5-Heptyl-2-(3-methylcyclohex-2-en-1-yl)benzene-1,3-diol
5-Hept-1-enyl-2-[(1R,6R)-3-methyl-6-prop-1-
en-2-ylcyclohex-2-en-1-yl]benzene-1,3-diol
5-Hept-1-enyl-4-[(1R,6R)-3-methyl-6-prop-1-en-
2-ylcyclohex-2-en-1-yl]benzene-1,3-diol
2-(6-Ethenyl-3-methylcyclohex-2-en-1-yl)-5-ethylbenzene-1,3-diol
4-(2,5-Dimethylheptan-4-yl)benzene-1,3-diol
6-[(E)-But-1-enyl]-3-(3-methyl-6-prop-1-en-2-
ylcyclohex-2-en-1-yl)benzene-1,2,4-triol
5-[2-(2,6-Dihydroxyphenyl)ethyl]-2-methylbenzene-1,3-diol
2-[4-(6-But-1-en-2-yl-3-methylcyclohex-2-
en-1-yl)-3,5-dihydroxyphenyl]acetonitrile
5-Methyl-2,4-bis(3-methylbut-2-enyl)benzene-1,3-diol
4-(3-Methylpent-2-en-2-yl)-6-propan-2-ylbenzene-1,3-diol
3-[(1R)-3-Methyl-6-prop-1-en-2-ylcyclohex-2-en-
1-yl]-6-[(E)-pent-1-enyl]benzene-1,2,4-triol
5-(2-Methyldecan-2-yl)benzene-1,3-diol
5-Ethyl-2-(3-methyl-6-prop-1-en-2-ylcyclohex-
2-en-1-yl)benzene-1,3-diol
5-Ethyl-2-[3-methyl-6-[1-(1-methyl-4-prop-1-en-2-ylcyclohex-
2-en-1-yl)oxyethenyl]cyclohex-2-en-1-yl]benzene-1,3-diol
4-(Hydroxymethyl)-2-[(1R,2S)-5-methyl-2-propan-
2-ylcyclohexyl]-5-pentylbenzene-1,3-diol
2-[(6S)-3-Methyl-6-prop-1-en-2-ylcyclohexen-
1-yl]-5-pentylbenzene-1,3-diol
5-Butyl-4-methylbenzene-1,3-diol
2-Ethyl-5-(2-phenylethynyl)benzene-1,3-diol
5-Fluoro-4-[(6R)-3-methyl-6-prop-1-en-2-ylcyclohex-
2-en-1-yl]benzene-1,3-diol
5-Ethynyl-4-[(6R)-3-methyl-6-prop-1-en-2-ylcyclohex-
2-en-1-yl]benzene-1,3-diol
5-[(E)-2-(4-Hydroxyphenyl)ethenyl]-4-[(6S)-3-methyl-6-
prop-1-en-2-ylcyclohex-2-en-1-yl]benzene-1,3-diol
(4Br,8aR)-6,9-dimethyl-4b,7,8,8a-tetrahydrophenanthrene-2,4-diol
5-[(E)-2-(4-Hydroxyphenyl)ethenyl]-2-(3-methyl-6-prop-
1-en-2-ylcyclohex-2-en-1-yl)benzene-1,3-diol
5-Ethynyl-4-(3-methyl-6-prop-1-en-2-
ylcyclohex-2-en-1-yl)benzene-1,3-diol
5-[(E)-2-(4-Hydroxyphenyl)ethenyl]-4-(3-methyl-
6-prop-1-en-2-ylcyclohex-2-en-1-yl)benzene-1,3-diol
2,4-Bis(3-methyl-6-prop-1-en-2-ylcyclohex-
2-en-1-yl)benzene-1,3,5-triol
2,5-Bis(prop-2-enyl)benzene-1,3-diol
2-Pentacos-5-enylbenzene-1,3-diol
2-Nonadec-5-enylbenzene-1,3-diol
2-Henicos-5-enylbenzene-1,3-diol
2-Tricos-5-enylbenzene-1,3-diol
2-Methyl-5-[(9E,11E)-pentadeca-9,11-dienyl]benzene-1,3-diol
5-[(9E,11E)-Pentadeca-9,11-dienyl]benzene-1,3-diol
5-(9,10,11,12-Tetraethylpentadecyl)benzene-1,3-diol
2-Methyl-5-(9,10,11,12-tetraethylpentadecyl)benzene-1,3-diol
5-[(7Z)-Pentadec-7-en-1-yl]benzene-1,3-diol
2-[4-(4-Hydroxyphenyl)cyclohexyl]-5-
(2-methyloctan-2-yl)benzene-1,3-diol
2-(5-Pentyltetradecyl)benzene-1,3-diol
5-(2-Methylheptan-2-yl)-2-[(1R,6R)-3-methyl-6-prop-
1-en-2-ylcyclohex-3-en-1-yl]benzene-1,3-diol
5-(2-Methylheptan-2-yl)-2-[(1R,6R)-3-methyl-6-prop-
1-en-2-ylcyclohex-2-en-1-yl]benzene-1,3-diol
[5-[3,5-Dihydroxy-4-[(1S,6S)-3-methyl-6-prop-1-en-2-
ylcyclohex-2-en-1-yl]phenyl]-5-methylhexyl] nitrate
[7-[3,5-Dihydroxy-4-[(1S,6S)-3-methyl-6-prop-1-en-2-
ylcyclohex-2-en-1-yl]phenyl]-7-methyloctyl] nitrate
[3-[3,5-Dihydroxy-4-[(1R,6R)-3-methyl-6-prop-1-en-
2-ylcyclohex-2-en-1-yl]phenyl]-3-methylbutyl] nitrate
4-[3,5-Dihydroxy-4-[(1R,6R)-3-methyl-6-prop-1-en-
2-ylcyclohex-2-en-1-yl]phenyl]butyl nitrate
3-[3,5-Dihydroxy-4-[(1S,6S)-3-methyl-6-prop-1-en-
2-ylcyclohex-2-en-1-yl]phenyl]propyl nitrate
[4-[3,5-Dihydroxy-4-[(1S,6S)-3-methyl-6-prop-1-en-2-
ylcyclohex-2-en-1-yl]phenyl]-4-methylpentyl] nitrate
7-[3,5-Dihydroxy-4-[(1R,6R)-3-methyl-6-prop-1-en-
2-ylcyclohex-2-en-1-yl]phenyl]heptyl nitrate
[6-[3,5-Dihydroxy-4-[(1S,6S)-3-methyl-6-prop-1-en-2-
ylcyclohex-2-en-1-yl]phenyl]-6-methylheptyl] nitrate
5-[3,5-Dihydroxy-4-[(1R,6R)-3-(hydroxymethyl)-6-prop-
1-en-2-ylcyclohex-2-en-1-yl]phenyl]pentyl nitrate
6-[3,5-Dihydroxy-4-[(1S,6S)-3-(hydroxymethyl)-6-prop-
1-en-2-ylcyclohex-2-en-1-yl]phenyl]hexyl nitrate
3-[3,5-Dihydroxy-4-[(1S,6S)-3-(hydroxymethyl)-6-prop-
1-en-2-ylcyclohex-2-en-1-yl]phenyl]propyl nitrate
4-[3,5-Dihydroxy-4-[(1S,6S)-3-(hydroxymethyl)-6-prop-
1-en-2-ylcyclohex-2-en-1-yl]phenyl]butyl nitrate
2-[(1R,6R)-3-(Hydroxymethyl)-6-prop-1-en-2-
ylcyclohex-2-en-1-yl]-5-propylbenzene-1,3-diol
2-[(1R,6S)-3-Methyl-6-prop-1-en-2-ylcyclohex-
2-en-1-yl]-5-propylbenzene-1,3-diol
4-Fluoro-2-[(6R)-3-methyl-6-prop-1-en-2-ylcyclohex-
2-en-1-yl]-5-pentylbenzene-1,3-diol
2-[(6R)-6-(1-Fluoroethenyl)-3-methylcyclohex-
2-en-1-yl]-5-methylbenzene-1,3-diol
4-Fluoro-5-methyl-2-[(6R)-3-methyl-6-prop-1-en-
2-ylcyclohex-2-en-1-yl]benzene-1,3-diol
2-(3-Methylbut-2-enyl)-5-[2-(4-methylphenyl)ethyl]benzene-1,3-diol
Cyclohexa-2,5-diene-1,4-dione; 2-[(1R,6R)-3-methyl-6-prop-
1-en-2-ylcyclohex-2-en-1-yl]-5-pentylbenzene-1,3-diol
5-[1-[(Z)-Hex-1-enyl]cyclopentyl]-2-[(2R,5S)-4,6,6-
trimethyl-2-bicyclo[3.1.1]hept-3-enyl]benzene-1,3-diol
2-[(1S,2S)-5-Hydroxy-2-prop-1-en-2-ylcyclohexyl]-
5-(2-methylheptan-2-yl)benzene-1,3-diol
5-(1-Hexylcyclopropyl)-2-[3-(hydroxymethyl)-6-prop-
1-en-2-ylcyclohex-2-en-1-yl]benzene-1,3-diol
5-(1-Hexylcyclopentyl)-2-[3-(hydroxymethyl)-6-prop-
1-en-2-ylcyclohex-2-en-1-yl]benzene-1,3-diol
2-(5-Hydroxy-2-prop-1-en-2-ylcyclohexyl)-
5-(2-methyloctan-2-yl)benzene-1,3-diol
2-[(5E)-5-Ethylidene-2-prop-1-en-2-ylcyclohexyl]-
5-(2-methyloctan-2-yl)benzene-1,3-diol

In certain embodiments, the molecule is any one of the molecules set forth in the table below:

Dronabinol
Cannabinol
Dexanabinol
HU-210
delta8-Tetrahydrocannabinol
Cannabichromene
(6Ar,10aR)-6,6,9-trimethyl-3-(2-phenylpropan-2-yl)-6a,7,10,10a-tetrahydrobenzo[c]chromen-1-ol
11-Hydroxytetrahydrocannabinol
Tetrahydrocannabivarin
delta8-THC
Unii-ggt8LM8A2O
Unii-8hhx5B1WP5
(6Ar,10aR)-3-(1-adamantyl)-6,6,9-trimethyl-6a,7,10,10a-tetrahydrobenzo[c]chromen-1-ol
CID 5311216
Synhexyl
delta9-Tetrahydrocannabinol
(6Ar,10aR)-9-(hydroxymethyl)-6,6-dimethyl-3-(2-methyloctan-2-yl)-6a,7,8,9,10,10a-hexahydrobenzo[c]chromen-1-ol
(6Ar,10aR)-6,6,9-trimethyl-3-[(2S,3R)-3-methyloctan-2-yl]-6a,7,10,10a-tetrahydrobenzo[c]chromen-1-ol
3-(5-Fluoropentyl)-6,6,9-trimethyl-6a,7,10,10a-tetrahydrobenzo[c]chromen-1-ol
(6Ar,10aR)-3-(1-hexylcyclopentyl)-6,6,9-trimethyl-6a,7,10,10a-tetrahydrobenzo[c]chromen-1-ol
(6Ar,10aR)-3-(6-bromo-2-methylhexan-2-yl)-6,6,9-trimethyl-6a,7,10,10a-tetrahydrobenzo[c]chromen-1-ol
Cannabinoids
(6Ar,10aR)-1-methoxy-6,6,9-trimethyl-3-(2-methyloctan-2-yl)-6a,7,10,10a-tetrahydrobenzo[c]chromene
11-Hydroxy-3-(1′,1′-dimethylheptyl)hexahydrocannabinol
11-Hydroxycannabinol
Cannabivarin
(6As,10aS)-6,6,9-trimethyl-3-(2-methyloctan-2-yl)-6a,7,10,10a-tetrahydrobenzo[c]chromen-1-ol
8-Hydroxy-delta(9)-tetrahydrocannabinol
Exo-Tetrahydrocannabinol
2′-Hydroxy-2,4,4,7,4′-pentamethylflavan
delta3-Tetrahydrocannabinol, (R)-
CID 53318931
(6Ar,10aR)-6,6,9-trimethyl-3-(2-methyloctan-2-yl)-6a,7,8,10a-tetrahydrobenzo[c]chromen-1-ol
(6Ar,10aR)-3-[2-(3-fluorophenyl)propan-2-yl]-6,6,9-trimethyl-6a,7,10,10a-tetrahydrobenzo[c]chromen-1-ol
3-(6-Bromohex-2-ynyl)-6,6,9-trimethyl-6a,7,10,10a-tetrahydrobenzo[c]chromen-1-ol
3-Hept-1-ynyl-6,6,9-trimethyl-6a,7,10,10a-tetrahydrobenzo[c]chromen-1-ol
(6Ar,10aR)-3-(2-cyclohexylpropan-2-yl)-6,6,9-trimethyl-6a,7,10,10a-tetrahydrobenzo[c]chromen-1-ol
6,6,9-Trimethyl-3-pentylbenzo[c]chromene-1,8-diol
Nabiximols
(6Ar,10aR)-6,6,9-trimethyl-3-[2-(4-methylphenyl)propan-2-yl]-6a,7,10,10a-tetrahydrobenzo[c]chromen-1-ol
(6Ar,10aR)-6,6,9-trimethyl-3-(2-methyloctan-2-yl)-6a,7,10,10a-tetrahydrobenzo[c]chromen-1-ol
4′-Methoxy-7-isoflavanol
3-(1,1-Dimethyl-heptyl)-9-hydroxymethyl-6,6-dimethyl-6a,7,10,10a-tetrahydro-6H-benzo[c]chromen-1-ol
THC, 11-OH-delta8
7-[(6Ar,10aR)-1-hydroxy-6,6,9-trimethyl-6a,7,10,10a-tetrahydrobenzo[c]chromen-3-yl]-7-methyloctanenitrile
Unii-52KD6XK4KF
3-(1,1-Dimethyl-heptyl)-6,6,9-trimethyl-6a,7,10,10a-tetrahydro-6H-benzo[c]chromen-1-ol
(6Ar,10aR)-3-(2-hexyl-1,3-dioxolan-2-yl)-6,6,9-trimethyl-6a,7,10,10a-tetrahydrobenzo[c]chromen-1-ol
(6Ar,10aR)-3-(2-cycloheptylpropan-2-yl)-6,6,9-trimethyl-6a,7,10,10a-tetrahydrobenzo[c]chromen-1-ol
Confluentin
(6Ar,10aR)-3-(1-hexylcyclopropyl)-6,6,9-trimethyl-6a,7,10,10a-tetrahydrobenzo[c]chromen-1-ol
Unii-EW1UQ3B1Y8
(6Ar,10aR)-3-(2-cyclopentylpropan-2-yl)-6,6,9-trimethyl-6a,7,10,10a-tetrahydrobenzo[c]chromen-1-ol
9-(Hydroxymethyl)-6,6-dimethyl-3-(2-methyloctan-2-yl)-6a,7,10,10a-tetrahydrobenzo[c]chromen-1-ol
6H-Dibenzo(b,d)pyran-1-ol,6a,7,8,10a-tetrahydro-6,6,9-trimethyl-3-pentyl-, (6aS-trans)-
11-Hydroxy-THC
Dimethylheptylpyran
(6Ar,10aR)-9-hydroperoxy-6,6-dimethyl-3-(2-methyloctan-2-yl)-6a,7,10,10a-tetrahydrobenzo[c]chromen-1-ol
(6Ar,10aR)-3-(1-hydroxy-2-methylpropan-2-yl)-6,6,9-trimethyl-6a,7,10,10a-tetrahydrobenzo[c]chromen-1-ol
2-Methyl-2-(4-methylpentyl)-3,4-dihydrochromen-5-ol
(6Ar,9R,10aR)-9-(hydroxymethyl)-6,6-dimethyl-3-(2-methyloctan-2-yl)-8,9-ditritio-7,8,10,10a-tetrahydro-6aH-
benzo[c]chromen-1-ol
9-Hexyl-9H-xanthene-3,6-diol
delta3-Tetrahydrocannabinol, (S)-
(6Ar,10aR)-3-[2-(4-ethylphenyl)propan-2-yl]-6,6,9-trimethyl-6a,7,10,10a-tetrahydrobenzo[c]chromen-1-ol
(6Ar,10aR)-6,6,9-trimethyl-3-(2-methylheptan-2-yl)-6a,7,10,10a-tetrahydrobenzo[c]chromen-1-ol
(6Ar,10aR)-6,6,9-trimethyl-3-(2-methylhexan-2-yl)-6a,7,10,10a-tetrahydrobenzo[c]chromen-1-ol
(6Ar,10aR)-6,6,9-trimethyl-3-(2-methylpentan-2-yl)-6a,7,10,10a-tetrahydrobenzo[c]chromen-1-ol
(6Ar,10aR)-3-tert-butyl-6,6,9-trimethyl-6a,7,10,10a-tetrahydrobenzo[c]chromen-1-ol
(6Ar,10aR)-3-[(E)-hept-1-enyl]-6,6,9-trimethyl-6a,7,10,10a-tetrahydrobenzo[c]chromen-1-ol
(6Ar,10aR)-3-[1-[(Z)-hex-1-enyl]cyclopentyl]-6,6,9-trimethyl-6a,7,10,10a-tetrahydrobenzo[c]chromen-1-ol
(6Ar,10aR)-3-[1-[(Z)-hex-1-enyl]cyclobutyl]-6,6,9-trimethyl-6a,7,10,10a-tetrahydrobenzo[c]chromen-1-ol
(6Ar,10aR)-3-(1-heptylcyclopropyl)-6,6,9-trimethyl-6a,7,10,10a-tetrahydrobenzo[c]chromen-1-ol
6,6,9-Trimethyl-6a,7,10,10a-tetrahydro-6H-benzo[c]chromen-1-ol
(E)-7-(1-Hydroxy-6,6,9-trimethyl-6a,7,10,10a-tetrahydrobenzo[c]chromen-3-yl)hept-5-enenitrile
3-[(E)-Hept-1-enyl]-6,6,9-trimethyl-6a,7,10,10a-tetrahydrobenzo[c]chromen-1-ol
6-[(6Ar,10aR)-1-hydroxy-6,6,9-trimethyl-6a,7,10,10a-tetrahydrobenzo[c]chromen-3-yl]-6-methylheptanenitrile
(6Ar,10aR)-6,6,9-trimethyl-3-nonyl-6a,7,10,10a-tetrahydrobenzo[c]chromen-1-ol
(6Ar,10aR)-3-(2,2-dichloro-1-hexylcyclopropyl)-6,6,9-trimethyl-6a,7,10,10a-tetrahydrobenzo[c]chromen-1-ol
(6Ar,10aR)-3-(2,2-dibromo-1-hexylcyclopropyl)-6,6,9-trimethyl-6a,7,10,10a-tetrahydrobenzo[c]chromen-1-ol
(6Ar)-6,6,9-trimethyl-3-(2-methyloctan-2-yl)-6a,7,8,10a-tetrahydrobenzo[c]chromen-1-ol
(6As,10aR)-3-(3-cyclopentylbutan-2-yl)-6,6,9-trimethyl-6a,7,10,10a-tetrahydrobenzo[c]chromen-1-ol
(6Ar,10aR)-3-(3-cyclopentylbutan-2-yl)-6,6,9-trimethyl-6a,7,10,10a-tetrahydrobenzo[c]chromen-1-ol
(6Ar,10aS)-3-(3-cyclopentylbutan-2-yl)-6,6,9-trimethyl-6a,7,10,10a-tetrahydrobenzo[c]chromen-1-ol
(6As,10aS)-3-(3-cyclopentylbutan-2-yl)-6,6,9-trimethyl-6a,7,10,10a-tetrahydrobenzo[c]chromen-1-ol
3-Heptyl-6,6,9-trimethyl-6a,7,10,10a-tetrahydro-6H-benzo[c]chromen-1-ol
(6Ar,10aR)-6,6-dimethyl-9-methylidene-3-(2-methyloctan-2-yl)-7,8,10,10a-tetrahydro-6aH-benzo[c]chromen-1-ol
(6Ar,10aR)-6,6,9-trimethyl-3-[2-(3-methylphenyl)propan-2-yl]-6a,7,10,10a-tetrahydrobenzo[c]chromen-1-ol
6,6-Dimethyl-3-(2-methyloctan-2-yl)benzo[c]chromene-1,9-diol
9-Methoxy-6,6-dimethyl-3-(2-methyloctan-2-yl)benzo[c]chromen-1-ol
(6Ar,8S,10aR)-6,6,9-trimethyl-3-pentyl-6a,7,8,10a-tetrahydrobenzo[c]chromene-1,8-diol
(6Ar,9S)-9-(hydroxymethyl)-6,6-dimethyl-3-(2-methyloctan-2-yl)-6a,7,8,9,10,10a-hexahydrobenzo[c]chromen-1-ol
(6Ar,9R)-9-(hydroxymethyl)-6,6-dimethyl-3-(2-methyloctan-2-yl)-6a,7,8,9,10,10a-hexahydrobenzo[c]chromen-1-ol
6H-Dibenzo(b,d)pyran, 6a,7,10,10a-tetrahydro-1-methoxy-6,6,9-trimethyl-3-pentyl-, (6aR-trans)-
[(6Ar,10aR)-6,6,9-trimethyl-3-pentyl-6a,7,10,10a-tetrahydrobenzo[c]chromen-1-yl] acetate
(6Ar,9R,10aR)-3-heptyl-9-(hydroxymethyl)-6,6-dimethyl-6a,7,8,9,10,10a-hexahydrobenzo[c]chromen-1-ol
(6Ar,10aR)-3-Hept-1-ynyl-6,6,9-trimethyl-6a,7,10,10a-tetrahydro-6H-benzo[c]chromen-1-ol
3-(1,1-Dimethyl-heptyl)-6,6,9-trimethyl-6H-benzo[c]chromen-1-ol
(6Ar,10aR)-9-(hydroxymethyl)-6,6-dimethyl-3-nonyl-6a,7,10,10a-tetrahydrobenzo[c]chromen-1-ol
(4Ar,12bR)-8-hexyl-2,5,5-trimethyl-1,4,4a,8,9,10,11,12b-octahydronaphtho[3,2-c]isochromen-12-ol
ClusiparalicolineC
(6Ar,10aR)-9-(hydroxymethyl)-6,6-dimethyl-3-[(2S,3R)-3-methyloctan-2-yl]-6a,7,10,10a-tetrahydrobenzo[c]chromen-1-ol
Unii-TJ6JD73J07
(10As)-9-(hydroxymethyl)-6,6-dimethyl-3-(2-methyloctan-2-yl)-6a,7,10,10a-tetrahydrobenzo[c]chromen-1-ol
(6As,10aS)-6,6,9-trimethyl-3-pentyl-6a,7,10,10a-tetrahydrobenzo[c]chromen-1-ol
(6Ar,10aR)-1-hydroxy-6,6-dimethyl-3-pentyl-7,8,10,10a-tetrahydro-6aH-benzo[c]chromen-9-one
4′-O-Methylequol
9-Hydroxy-9-norhexahydrocannabinol
(+)-6,6,9-Trimethyl-3-pentyl-6a,7,8,10a-tetrahydro-6H-benzo[c]chromen-1-ol
O-Methyl-delta-9 tetrahydrocannabinol
7,8,9,10-Tetrahydro-3-(1,2-dimethylheptyl)-6,6,10-trimethyl-6H-dibenzo(b,d)pyran-1-ol
Cyclopenta(c)(1)benzopyran-9-ol, 1,2,3,4-tetrahydro-7-(1,2-dimethylheptyl)-2,2,4,4-tetramethyl-
1,2,3,4-Tetrahydro-7-(1,2-dimethylheptyl)-2,4,4-trimethylcyclopenta(c)(1)benzopyran-9-ol
1,2,3,4-Tetrahydro-7-(1,2-dimethylheptyl)-1,4,4-trimethylcyclopenta(c)(1)benzopyran-9-ol
Dimethyl-heptyl tetrahydrocannabinol
8-Hydroxy-delta-9-THC
Hexahydrocannabinol
1,2,3,4-Tetrahydro-4,4-dimethyl-7-(1,2-dimethylheptyl)cyclopenta(c)(1)benzopyran-9-ol
Cannabidiol-3-monomethyl ether
1alpha,2alpha-Epoxyhexahydrocannabinol
Tinabinol
3-[5-(4-Fluorophenyl)-3-methylpentan-2-yl]-6,6,9-trimethyl-7,8,9,10-tetrahydrobenzo[c]chromen-1-ol
7,8,9,10-Tetrahydro-3-(1-methyl-4-phenylbutyl)-6,6,9-trimethyl-6H-dibenzo(b,d)pyran-1-ol
Tetrahydrocannabivarol
(8Ar,10aR)-8,8,10a-trimethyl-3-pentyl-6,7,8a,9-tetrahydro-5H-xanthen-1-ol
2-Methyl-2-[4-methyl-4-(4-methylphenyl)pentyl]-7-pentyl-3,4-dihydrochromen-5-ol
(6Ar,9S,10aR)-6,6,9-trimethyl-3-pentyl-10,10a-dihydro-6aH-benzo[c]chromene-1,9-diol
Artocarbene
(1R,10R,13R,15S)-9,9,13-Trimethyl-5-pentyl-8,14-dioxatetracyclo[8.5.0.02,7.013,15]pentadeca-2,4,6-trien-3-ol
(6Ar,10R,10aR)-6,6-dimethyl-9-methylidene-3-pentyl-7,8,10,10a-tetrahydro-6aH-benzo[c]chromene-1,10-diol
(6R)-6,9,9-Trimethyl-3-pentyl-5,6,7,8-tetrahydroxanthen-1-ol
(6S)-6,9,9-Trimethyl-3-pentyl-5,6,7,8-tetrahydroxanthen-1-ol
(6R)-2-Ethyl-6,9,9-trimethyl-5,6,7,8-tetrahydroxanthen-3-ol
(6Ar,10S,10aR)-6,6,9-trimethyl-3-pentyl-6a,7,10,10a-tetrahydrobenzo[c]chromene-1,10-diol
(6Ar,10R,10aR)-6,6,9-trimethyl-3-pentyl-6a,7,10,10a-tetrahydrobenzo[c]chromene-1,10-diol
(8Ar,10aS)-8,8,10a-trimethyl-3-pentyl-6,7,8a,9-tetrahydro-5H-xanthen-1-ol
(6Ar,10aR)-3-(1-adamantyl)-9-(hydroxymethyl)-6,6-dimethyl-6a,7,10,10a-tetrahydrobenzo[c]chromen-1-ol
8 alpha,9 alpha-Epoxyhexahydrocannabinol
(6Ar,10aR)-6,6-dimethyl-3-(2-methylpentan-2-yl)-6a,7,10,10a-tetrahydrobenzo[c]chromen-9-ol
(6Ar,10aR)-6,6,9-trimethyl-3-octan-2-yl-6a,7,10,10a-tetrahydrobenzo[c]chromen-1-ol
(6Ar,10aR)-3-(4-bromobutyl)-6,6,9-trimethyl-6a,7,8,10a-tetrahydrobenzo[c]chromen-1-ol
(6Ar,10aR)-3-[2-(4-fluorophenyl)propan-2-yl]-6,6,9-trimethyl-6a,7,10,10a-tetrahydrobenzo[c]chromen-1-ol
(6Ar,10aR)-6,6,9-trimethyl-3-[2-(4-propylphenyl)propan-2-yl]-6a,7,10,10a-tetrahydrobenzo[c]chromen-1-ol
(6Ar,10aR)-3-[2-(3,5-dimethylphenyl)propan-2-yl]-6,6,9-trimethyl-6a,7,10,10a-tetrahydrobenzo[c]chromen-1-ol
(6Ar,10aR)-6,6,9-trimethyl-3-[2-(2-methylphenyl)propan-2-yl]-6a,7,10,10a-tetrahydrobenzo[c]chromen-1-ol
(6Ar,10aR)-6,6,9-trimethyl-3-[(1S,2S,4S)-1,7,7-trimethyl-2-bicyclo[2.2.1]heptanyl]-6a,7,10,10a-tetrahydrobenzo[c]chromen-1-ol
(6Ar,10aR)-6,6,9-trimethyl-3-[(1S,2R,4S)-1,7,7-trimethyl-2-bicyclo[2.2.1]heptanyl]-6a,7,10,10a-tetrahydrobenzo[c]chromen-1-ol
(6Ar,10aR)-6,6,9-trimethyl-3-phenoxy-6a,7,10,10a-tetrahydrobenzo[c]chromen-1-ol
(6Ar,10aR)-6,6,9-trimethyl-3-(4-methylpentyl)-6a,7,10,10a-tetrahydrobenzo[c]chromen-1-ol
(6Ar,10aR)-3-[1-[(Z)-hex-1-enyl]cyclohexyl]-6,6,9-trimethyl-6a,7,10,10a-tetrahydrobenzo[c]chromen-1-ol
(6Ar,10aR)-3-[1-[(Z)-hex-1-enyl]cyclopropyl]-6,6,9-trimethyl-6a,7,10,10a-tetrahydrobenzo[c]chromen-1-ol
(6Ar,10aR)-3-(1-heptylcyclohexyl)-6,6,9-trimethyl-6a,7,10,10a-tetrahydrobenzo[c]chromen-1-ol
(6Ar,10aR)-3-(1-heptylcyclopentyl)-6,6,9-trimethyl-6a,7,10,10a-tetrahydrobenzo[c]chromen-1-ol
(6Ar,10aR)-3-(1-heptylcyclobutyl)-6,6,9-trimethyl-6a,7,10,10a-tetrahydrobenzo[c]chromen-1-ol
(6Ar,10aR)-3-(1-hexylcyclohexyl)-6,6,9-trimethyl-6a,7,10,10a-tetrahydrobenzo[c]chromen-1-ol
(6Ar,10aR)-3-(1-hexylcyclobutyl)-6,6,9-trimethyl-6a,7,10,10a-tetrahydrobenzo[c]chromen-1-ol
(6Ar,10aR)-6,6,9-trimethyl-3-(4-propylphenyl)-6a,7,10,10a-tetrahydrobenzo[c]chromen-1-ol
1-[(6Ar,10aR)-1-hydroxy-6,6,9-trimethyl-6a,7,10,10a-tetrahydrobenzo[c]chromen-3-yl]cyclopentane-1-carbonitrile
(6Ar,10aR)-6,6,9-trimethyl-3-(4-methylphenyl)-6a,7,10,10a-tetrahydrobenzo[c]chromen-1-ol
(6Ar,10aR)-3-(4-ethylphenyl)-6,6,9-trimethyl-6a,7,10,10a-tetrahydrobenzo[c]chromen-1-ol
6-Allyl-4,6,7,10-tetramethyl-6H-benzo[c]chromene-3,8-diol
6,6,9-Trimethyl-3-[(E)-oct-4-enyl]-6a,7,10,10a-tetrahydrobenzo[c]chromen-1-ol
3-[(E)-6-Bromohex-2-enyl]-6,6,9-trimethyl-6a,7,10,10a-tetrahydrobenzo[c]chromen-1-ol
3-Hepta-1,6-diynyl-6,6,9-trimethyl-6a,7,10,10a-tetrahydrobenzo[c]chromen-1-ol
6,6,9-Trimethyl-3-oct-4-ynyl-6a,7,10,10a-tetrahydrobenzo[c]chromen-1-ol
6-(1-Hydroxy-6,6,9-trimethyl-6a,7,10,10a-tetrahydrobenzo[c]chromen-3-yl)hex-5-ynenitrile
Methyl4-(1-hydroxy-6,6,9-trimethyl-6a,7,10,10a-tetrahydrobenzo[c]chromen-3-yl)but-2-ynoate
6,6,9-Trimethyl-3-non-2-ynyl-6a,7,10,10a-tetrahydrobenzo[c]chromen-1-ol
3-But-3-ynyl-6,6,9-trimethyl-6a,7,10,10a-tetrahydrobenzo[c]chromen-1-ol
1-Methoxy-6,6,9-trimethyl-3-oct-2-ynyl-6a,7,10,10a-tetrahydrobenzo[c]chromene
3-[(E)-6-Fluorohex-2-enyl]-6,6,9-trimethyl-6a,7,10,10a-tetrahydrobenzo[c]chromen-1-ol
6,6,9-Trimethyl-3-oct-3-ynyl-6a,7,10,10a-tetrahydrobenzo[c]chromen-1-ol
3-(5-Hydroxypent-2-ynyl)-6,6,9-trimethyl-6a,7,10,10a-tetrahydrobenzo[c]chromen-1-ol
6,6,9-Trimethyl-3-[(E)-oct-3-enyl]-6a,7,10,10a-tetrahydrobenzo[c]chromen-1-ol
3-(4-Bromopent-2-ynyl)-6,6,9-trimethyl-6a,7,10,10a-tetrahydrobenzo[c]chromen-1-ol
6,6,9-Trimethyl-3-octa-2,7-diynyl-6a,7,10,10a-tetrahydrobenzo[c]chromen-1-ol
6-(1-Hydroxy-6,6,9-trimethyl-6a,7,10,10a-tetrahydrobenzo[c]chromen-3-yl)hex-4-ynenitrile
3-(4-Bromobut-2-ynyl)-6,6,9-trimethyl-6a,7,10,10a-tetrahydrobenzo[c]chromen-1-ol
3-[(E)-6-Hydroxyhex-2-enyl]-6,6,9-trimethyl-6a,7,10,10a-tetrahydrobenzo[c]chromen-1-ol
3-[(E)-6-Methoxyhex-2-enyl]-6,6,9-trimethyl-6a,7,10,10a-tetrahydrobenzo[c]chromen-1-ol
6,6,9-Trimethyl-3-[(E)-oct-2-enyl]-6a,7,10,10a-tetrahydrobenzo[c]chromen-1-ol
(6Ar)-6,6,9-trimethyl-3-pentyl-6a,7,8,9,10,10a-hexahydrobenzo[c]chromen-1-ol
(6As)-3-(4-azidobutyl)-6,6,9-trimethyl-6a,7,8,10a-tetrahydrobenzo[c]chromen-1-ol
(6Ar,10aR)-3-benzyl-6,6,9-trimethyl-6a,7,10,10a-tetrahydrobenzo[c]chromen-1-ol
(6Ar,10aR)-3-(5-azidopentyl)-6,6,9-trimethyl-6a,7,10,10a-tetrahydrobenzo[c]chromen-1-ol
6,6,9-Trimethyl-3-(5,5,5-trifluoropentyl)-6a,7,10,10a-tetrahydrobenzo[c]chromen-1-ol
7-[(E)-2-(4-Hydroxyphenyl)ethenyl]-2,2-dimethylchromen-5-ol
Perrottetinene
(6Ar,10aR)-6,6,9-trimethyl-3-(5,5,5-trifluoropentyl)-6a,7,10,10a-tetrahydrobenzo[c]chromen-1-ol
(6Ar,10aR)-9-(fluoromethyl)-6,6-dimethyl-3-pentyl-6a,7,10,10a-tetrahydro-6H-benzo[c]chromen-1-ol
delta1-Tetrahydrocannabiorcol
9-Hydroxymethyl-6,6-dimethyl-3-pentyl-6a,7,10,10a-tetrahydro-6H-benzo[c]chromen-1-ol
delta-9-cis-Tetrahydrocannabinol, (−)-
(6S,6Ar,9R,10aR)-6-ethyl-9-(hydroxymethyl)-6-methyl-3-(2-methyloctan-2-yl)-6a,7,8,9,10,10a-hexahydrobenzo[c]chromen-1-ol
(6Ar,10aR)-3-(1-hydroxyheptyl)-6,6,9-trimethyl-6a,7,10,10a-tetrahydrobenzo[c]chromen-1-ol
1-[(6Ar,10aR)-1-hydroxy-6,6,9-trimethyl-6a,7,10,10a-tetrahydrobenzo[c]chromen-3-yl]cyclohexane-1-carbonitrile
3-[1,3]Dithiolan-2-yl-6,6,9-trimethyl-6a,7,10,10a-tetrahydro-6H-benzo[c]chromen-1-ol
Cannabitriol
(6Ar,10aR)-3-(2-adamantyl)-6,6,9-trimethyl-6a,7,10,10a-tetrahydrobenzo[c]chromen-1-ol
(6Ar,10aR)-3-(1-adamantylmethyl)-6,6,9-trimethyl-6a,7,10,10a-tetrahydrobenzo[c]chromen-1-ol
(6Ar,10aR)-3-(2-adamantylmethyl)-6,6,9-trimethyl-6a,7,10,10a-tetrahydrobenzo[c]chromen-1-ol
(6Ar,10aR)-3-(2-adamantylidenemethyl)-6,6,9-trimethyl-6a,7,10,10a-tetrahydrobenzo[c]chromen-1-ol
(6S,6Ar,9R,10aR)-9-(hydroxymethyl)-6-(2-iodoethyl)-6-methyl-3-(2-methyloctan-2-yl)-6a,7,8,9,10,10a-
hexahydrobenzo[c]chromen-1-ol
(6Ar,9R,10aR)-3-hept-1-ynyl-9-(hydroxymethyl)-6,6-dimethyl-6a,7,8,9,10,10a-hexahydrobenzo[c]chromen-1-ol
(6S,6Ar,9R,10aR)-9-(hydroxymethyl)-6-methyl-3-(2-methyloctan-2-yl)-6-propyl-6a,7,8,9,10,10a-hexahydrobenzo[c]chromen-1-ol
3-(1,1-Dimethyl-butyl)-6,6,9-trimethyl-6H-benzo[c]chromen-1-ol
(6S,6Ar,9R,10aR)-6-(2-hydroxyethyl)-9-(hydroxymethyl)-6-methyl-3-(2-methyloctan-2-yl)-6a,7,8,9,10,10a-
hexahydrobenzo[c]chromen-1-ol
(6S,6Ar,9R,10aR)-9-(hydroxymethyl)-6-(3-iodopropyl)-6-methyl-3-(2-methyloctan-2-yl)-6a,7,8,9,10,10a-
hexahydrobenzo[c]chromen-1-ol
(6Ar,10aR)-6,6,9-trimethyl-3-oct-1-en-2-yl-6a,7,10,10a-tetrahydrobenzo[c]chromen-1-ol
(6R,6Ar,9R,10aR)-6,9-bis(hydroxymethyl)-6-methyl-3-(2-methyloctan-2-yl)-6a,7,8,9,10,10a-hexahydrobenzo[c]chromen-1-ol
6,6,9-Trimethyl-3-oct-2-ynyl-6a,7,10,10a-tetrahydrobenzo[c]chromen-1-ol
(4Ar,12bR)-2,5,5-trimethyl-9-pentyl-1,4,4a,8,9,10,11,12b-octahydronaphtho[3,2-c]isochromen-12-ol
3-Hex-2-ynyl-6,6,9-trimethyl-6a,7,10,10a-tetrahydrobenzo[c]chromen-1-ol
Machaeriol A
(6Ar,10aR)-1-hexyl-6,6,9-trimethyl-1,2,3,4,6a,7,10,10a-octahydronaphtho[1,2-c]isochromen-11-ol
1-Methoxy-6,6,9-trimethyl-3-(2-methyloctan-2-yl)-6a,7,10,10a-tetrahydrobenzo[c]chromene
Butyl-delta(9)-tetrahydrocannabinol
Butyl-delta(8)-tetrahydrocannabinol
Ethyl-delta-8-tetrahydrocannabinol
Cannabichromevarin
(6Ar)-6,6,9-trimethyl-3-pentyl-6a,7,10,10a-tetrahydrobenzo[c]chromen-1-ol
Propyl-delta(8)-tetrahydrocannabinol
Canbisol, (+/-)-(rrr)-
(9R)-3,6,6,9-Tetramethyl-7,8,9,10-tetrahydrobenzo[c]chromen-1-ol
3,6,6,9-Tetramethyl-7,8,9,10-tetrahydro-6H-benzo[c]chromen-1-ol
Tetrahydrocannabinol acetate
Abn-delta(8)-thc
2H-1-Benzopyran-5-ol,2,2-dimethyl-7-pentyl-
11-Hydroxy-delta8-tetrahydrocannabinol
9-Hydroxyhexahydrocannabinol
n-Hexyl-delta-9-thc
6a,7,10,10a-Tetrahydro-1-hydroxy-6,6-dimethyl-3-pentyl-6H-dibenzo(b,d)pyran-9(8H)-one
ABN-delta8-THC
6H-Dibenzo(b,d)pyran-1-ol,6a,7,10,10a-tetrahydro-3,6,6,9-tetramethyl-
Canbisol
3-[6-(4-Fluorophenyl)-3-methylhexan-2-yl]-6,6,9-trimethyl-7,8,9,10-tetrahydrobenzo[c]chromen-1-ol
3-[6-(4-Fluorophenyl)hexan-2-yl]-6,6,9-trimethyl-7,8,9,10-tetrahydrobenzo[c]chromen-1-ol
8beta-Hydroxy-dleta(9)-THC
Methyloctylpyran
11-Hydroxy-delta(8)-thc
6H-DIBENZO(b,d)PYRAN-3-OL, 6,6-DIMETHYL-7,8,9,10-TETRAHYDRO-
6H-Dibenzo(b,d)pyran-1-ol, 6a-beta,7,8,10-a-beta-tetrahydro-3-pentyl-6,6,9-trimethyl-, (+−)-Z-
(7R,8Ar,10aR)-7-iodo-8,8,10a-trimethyl-3-pentyl-6,7,8a,9-tetrahydro-5H-xanthen-1-ol
(2R)-2-(4,8-Dimethylnona-3,7-dienyl)-2,7-dimethylchromen-5-ol
(7S)-6,6,9-Trimethyl-3-pentyl-6a,7,8,10a-tetrahydrobenzo[c]chromene-1,7-diol
2-[(3Z)-4,8-Dimethylnona-3,7-dienyl]-2,7-dimethylchromen-5-ol
(6R)-6,9,9-Trimethyl-5,6,7,8-tetrahydroxanthen-3-ol
2,7-Dimethyl-8-(3-methylbut-2-enyl)-2-[(1E)-4-methylpenta-1,3-dienyl]-3,4-dihydrochromen-5-ol
(6Ar,9S,10aR)-6,6,9-trimethyl-3-pentyl-7,8,10,10a-tetrahydro-6aH-benzo[c]chromene-1,9-diol
(6Ar,10aR)-3-(6-azidohex-2-ynyl)-6,6,9-trimethyl-6a,7,8,10a-tetrahydrobenzo[c]chromen-1-ol
(6Ar,8R,9R,10aR)-9-(hydroxymethyl)-6,6-dimethyl-3-(2-methyloctan-2-yl)-8,9-ditritio-7,8,10,10a-tetrahydro-6aH-
benzo[c]chromen-1-ol
delta-7-cis-Isotetrahydrocannabivarin
7-Pentyl-2H-1-benzopyran-5-ol
Canbisol, (+/−)-(rrs)-
(6Ar,10aR)-3-[(3R)-2,3-dimethylpentan-2-yl]-1-methoxy-6,6,9-trimethyl-6a,7,10,10a-tetrahydrobenzo[c]chromene
(2S)-2-[(3E)-4,8-Dimethylnona-3,7-dienyl]-2,7-dimethylchromen-5-ol
(6As,10aS)-9-(hydroxymethyl)-6,6-dimethyl-3-(2-methylhexan-2-yl)-6a,7,10,10a-tetrahydrobenzo[c]chromen-1-ol
3-(6-Aminohex-2-ynyl)-6,6,9-trimethyl-6a,7,10,10a-tetrahydrobenzo[c]chromen-1-ol
Methyl 5-(1-hydroxy-6,6,9-trimethyl-6a,7,10,10a-tetrahydrobenzo[c]chromen-3-yl)pent-2-ynoate
6,6-Dimethyl-3-pentyl-7,8,9,10-tetrahydrobenzo[c]chromen-1-ol
(6Ar,8R,10aR)-6,6-dimethyl-9-methylidene-3-pentyl-7,8,10,10a-tetrahydro-6aH-benzo[c]chromene-1,8-diol
(10As)-3-tert-butyl-9-(hydroxymethyl)-6,6-dimethyl-6a,7,10,10a-tetrahydrobenzo[c]chromen-1-ol
9-(Hydroxymethyl)-6,6-dimethyl-3-(2-methyloctan-2-yl)-6a,7,8,9,10,10a-hexahydrobenzo[c]chromen-1-ol
6-(Hydroxymethyl)-6,9-dimethyl-3-pentyl-6a,7,8,10a-tetrahydrobenzo[c]chromen-1-ol
6H-Dibenzo(b,d)pyran-1,9-diol, 6a,7,8,9,10,10a-hexahydro-6,6-dimethyl-3-(4-methoxy-1-methylbutyl)-
6H-Dibenzo(b,d)pyran-1,9-diol, 6a,7,8,9,10,10a-hexahydro-6,6-dimethyl-3-(4-ethoxy-1-methylbutyl)-
delta8-Tetrahydrocannabinol, (6as-cis)-
delta(Sup8)-Thc sulfate potassium salt
7-[4-(4-Fluorophenyl)-1-methylbutyl]-1,2,3,4-tetrahydro-4,4-dimethylcyclopenta[c][1]benzopyran-9-ol
2,2-Dimethyl-4-phenyl-3,4-dihydro-2H-1-benzopyran-7-ol
(6Ar,10aR)-6,6-dimethyl-9-methylidene-3-propyl-7,8,10,10a-tetrahydro-6aH-benzo[c]chromen-1-ol
6,6-Dimethyl-9-methylidene-3-pentyl-7,8,10,10a-tetrahydro-6aH-benzo[c]chromen-1-ol
(6Ar,10aR)-3-(5-bromopentyl)-6,6,9-trimethyl-6a,7,10,10a-tetrahydrobenzo[c]chromen-1-ol
2,2-Dimethylchroman-7-ol
Unii-npu5swp5ZS
5-Methoxy-2,2-dimethyl-7-[2-(4-hydroxyphenyl)ethenyl]-2H-1-benzopyran
(10As)-9-(hydroxymethyl)-6,6-dimethyl-3-(2-methylpentan-2-yl)-6a,7,10,10a-tetrahydrobenzo[c]chromen-1-ol
7-[(6Ar,10aR)-1-hydroxy-6,6,9-trimethyl-6a,7,10,10a-tetrahydrobenzo[c]chromen-3-yl]hept-5-ynenitrile
6,6,9-Trimethyl-3-pentyl-7,8,9,10-tetrahydrobenzo[c]chromen-1-ol
4′-Hydroxy-thc
3-Heptyl-delta(1)-tetrahydrocannabinol
2-(2,3-Dihydro-7-ethyl-2,4,4-trimethyl-4H-1-benzopyran-2-YL)-5-ethylphenol
3-Pehhdo
(6Ar,8S,9R,10aR)-9-(hydroxymethyl)-6,6-dimethyl-3-(2-methyloctan-2-yl)-8,9-ditritio-7,8,10,10a-tetrahydro-6aH-
benzo[c]chromen-1-ol
1,5-Dihydro-5,5-dimethyl-8-(1,2-dimethylheptyl)-2H,4H-thiopyrano(3,4-c)(1)benzopyran-10-ol
Ethyl-delta-9-tetrahydrocannabinol
3′,11-Dihydroxy-thc
3′-Hydroxy-thc
4-(7-Methoxychroman-3-yl)phenol
6H-Dibenzo(b,d)pyran-1-ol, 3-hexyl-6a,7,10,10a-tetrahydro-6,6,9-trimethyl-, (6aR-trans)-
12-(2-Hydroxypropan-2-yl)-9-methyl-5-propyl-8-oxatricyclo[7.3.1.02,7]trideca-2,4,6-trien-3-ol
6H-Dibenzo(b,d)pyran-1,9,10-triol, 7,8,9,10-tetrahydro-6,6,9-trimethyl-3-pentyl-,trans-
2-IA-8-Thc
2′-Hydroxy-delta(9)-thc
7-Hydroxyhexahydrocannabinol
5′-Tma-thc
6,6,9-Trimethyl-1-pentyl-6a,7,8,10a-tetrahydro-6h-benzo[c]chromen-3-ol
6,6,9-Trimethyl-4-pentyl-6a,7,8,10a-tetrahydro-6h-benzo[c]chromen-1-ol
2,2-Dimethyl-4,7-di(propan-2-yl)-3,4-dihydro-2h-chromen-6-ol
6H-DIBENZO(b,d)PYRAN-3-OL, 6,6-DIMETHYL-2-HEXYL-7,8,9,10-TETRAHYDRO-
6H-DIBENZO(b,d)PYRAN-1,3-DIOL, 6,6-DIMETHYL-7,8,9,10-TETRAHYDRO-
(6Ar,10aR)-6,6-dimethyl-3-pentyl-6a,7,10,10a-tetrahydrobenzo[c]chromen-1-ol
2-Methyl-2-(4-methylpent-3-enyl)-7-[(E)-2-phenylethenyl]chromen-5-ol
2-[(3E)-4,8-Dimethylnona-3,7-dienyl]-2-methyl-7-[(E)-2-phenylethenyl]chromen-5-ol
(6Ar,10aR)-3-hexyl-6,6,9-trimethyl-1,2,3,4,6a,7,10,10a-octahydronaphtho[1,2-c]isochromen-11-ol
8,11-Dihydroxy-delta9-tetrahydrocannabinol
4-(2,2,4-Trimethyl-3H-chromen-4-yl)benzene-1,3-diol
(9R)-4,6,6,9-Tetramethyl-7,8,9,10-tetrahydrobenzo[c]chromen-3-ol
(9R)-2-Ethyl-6,6,9-trimethyl-7,8,9,10-tetrahydrobenzo[c]chromen-3-ol
(6R)-3,6,9,9-Tetramethyl-5,6,7,8-tetrahydroxanthen-1-ol
2,2-Dimethyl-7-[(E)-styryl]-2H-1-benzopyran-5-ol
(9R)-6,6,9-Trimethyl-7,8,9,10-tetrahydrobenzo[c]chromen-3-ol
delta4-Tetrahydrocannabinol, 1R-trans-
[(6As,10aS)-6,6,9-trimethyl-3-pentyl-6a,7,8,10a-tetrahydrobenzo[c]chromen-1-yl]oxy-trimethylsilane
6,6-Dimethyl-3-pentylbenzo[c]chromene-1,9-diol
4-(7-Butoxy-3,4-dihydro-2H-chromen-3-yl)phenol
(1″,2″-Dimethyl-5″-ethyl)-delta6-tetrahydrocannabinol
AlbatrelinC
1,3-Benzenediol, 4-(3,4-dihydro-2,4,4-trimethyl-2H-1-benzopyran-2-yl)-
6,6,9-Trimethyl-3-(2-phenylpropan-2-yl)-6a,7,10,10a-tetrahydro-6H-dibenzo[b,d]pyran-1-ol
10-O-Ethylcannabitriol
(6Ar,10aR)-6,6,9-trimethyl-6a,7,10,10a-tetrahydrobenzo[c]chromene-1,3-diol
1-O-Methyl-11-hydroxy-delta-8-tetrahydrocannabinol
2,3,3,4,4-Pentamethyl-2-phenylchromen-5-ol
1-Methoxy-6,6,9-trimethyl-3-(2-methylpentan-2-yl)-6a,7,10,10a-tetrahydrobenzo[c]chromene
1-Methoxy-6,6,9-trimethyl-3-(2-methylbutan-2-yl)-6a,7,10,10a-tetrahydrobenzo[c]chromene
3-Tert-butyl-1-methoxy-6,6,9-trimethyl-6a,7,10,10a-tetrahydrobenzo[c]chromene
[1-Methoxy-6,6-dimethyl-3-(2-methylheptan-2-yl)-6a,7,10,10a-tetrahydrobenzo[c]chromen-9-yl]methanol
3-Heptyl-9-(hydroxymethyl)-6,6-dimethyl-6a,7,8,9,10,10a-hexahydrobenzo[c]chromen-1-ol
12-Methyl-5-(3-methyloctan-2-yl)-9-propan-2-yl-8-oxatricyclo[7.3.1.02,7]trideca-2,4,6-triene-3,11-diol
(1S,9R,12R)-12-Methyl-5-(3-methyloctan-2-yl)-9-propan-2-yl-8-oxatricyclo[7.3.1.02,7]trideca-2,4,6-triene-3,11-diol
(6Ar)-9-(hydroxymethyl)-6,6-dimethyl-3-pentyl-6a,7,8,10a-tetrahydrobenzo[c]chromen-1-ol
(6Ar,8R)-6,6,9-trimethyl-3-pentyl-6a,7,8,10a-tetrahydrobenzo[c]chromene-1,8-diol
(6Ar)-6,6,9-trimethyl-3-(3-methyloctan-2-yl)-6a,7,10,10a-tetrahydrobenzo[c]chromen-1-ol
(6Ar)-3-(5-hydroxypentyl)-6,6,9-trimethyl-6a,7,8,10a-tetrahydrobenzo[c]chromen-1-ol
(6Ar,8S)-6,6,9-trimethyl-3-pentyl-6a,7,8,10a-tetrahydrobenzo[c]chromene-1,8-diol
2-Methyl-2-(3-hydroxy-4-methyl-4-pentenyl)-7-pentyl-2H-1-benzopyran-5-ol
[(6Ar,10aR)-6,6,9-trimethyl-3-pentyl-6a,7,8,10a-tetrahydrobenzo[c]chromen-1-yl]oxy-trimethylsilane
2,7-Dimethyl-2-(4-methyl-3-pentenyl)-2H-1-benzopyran-5-ol
5,5-Dimethyl-8-(3-methyloctan-2-yl)-2,4-dihydro-1H-thiopyrano[3,4-c]chromen-10-ol
3-[5-(4-Fluorophenyl)pentan-2-yl]-6,6,9-trimethyl-7,8,9,10-tetrahydrobenzo[c]chromen-1-ol
10-Methyl-3-(2-methyloctan-2-yl)-6,7,8,9,10,11-hexahydrobenzo[d][1]benzoxepin-1-ol
7,7,10-Trimethyl-3-(2-methyloctan-2-yl)-8,9,10,11-tetrahydro-6H-benzo[d][1]benzoxepin-1-ol
6,6,10-Trimethyl-3-(2-methyloctan-2-yl)-8,9,10,11-tetrahydro-7H-benzo[d][1]benzoxepin-1-ol
THC-O-phosphate
2H-1-Benzopyran-7-ol, 3,4-dihydro-2,2-dimethyl-4-(1-methylethyl)-
7-Cyclopentyl-3,4-dihydro-4-isopropyl-2,2-dimethyl-2H-1-benzopyran-4-ol
(6Ar,10aR)-6,6,9-trimethyl-3-pentyl-6a,7,8,9,10,10a-hexahydrobenzo[c]chromen-1-ol
6,6,9-Trimethyl-3-(2-phenylethyl)-6a,7,8,10a-tetrahydrobenzo[c]chromen-1-ol
2,2-Dimethylchroman-5-ol
(6Ar,10aR)-6,6,9-trimethyl-3-(5,5,5-trideuteriopentyl)-6a,7,8,10a-tetrahydrobenzo[c]chromen-1-ol
3,4-Dihydro-5-hydroxy-7-methoxy-2,2-dimethyl-2H-1-benzopyran
2,2-Dimethyl-5,7-chromanediol
(6Ar)-3-[(Z)-6-azidohex-2-enyl]-6,6,9-trimethyl-6a,7,10,10a-tetrahydrobenzo[c]chromen-1-ol
Unii-cgygfrrxkk
(2R)-2-[(3E,6Z)-8-Hydroxy-4,8-dimethylnona-3,6-dienyl]-2,7-dimethylchromen-5-ol
2-Methyl-2-[(3E,7E)-4,8,12-trimethyltrideca-3,7,11-trienyl]-3,4-dihydrochromen-5-ol
2-Methyl-2-[(3E,7E)-4,8,12-trimethyltrideca-3,7,11-trienyl]-3,4-dihydrochromen-7-ol
delta5-Tetrahydrocannabinol, (1S,3R,4R)-
(2R)-2-[(3E)-7-Hydroxy-4,8-dimethylnona-3,8-dienyl]-2,7-dimethylchromen-5-ol
Machaeriol C
1-Methoxy-6,6-dimethyl-9-methylidene-3-(2-methyloctan-2-yl)-7,8,10,10a-tetrahydro-6aH-benzo[c]chromene
2,2,8,8-Tetramethyl-3,4,6,7-tetrahydropyrano[3,2-g]chromen-5-ol
1-Hydroxy-6,6-dimethyl-3-pentyl-7,8-dihydro-6aH-benzo[c]chromen-9-one
6,6,9-Trimethyl-3-(3-methyloctan-2-yl)-6a,7,8,10a-tetrahydro-6h-benzo[c]chromen-1-ol
6,6,9-Trimethyl-7,8,9,10-tetrahydrobenzo[c]chromen-3-ol
2-Methyl-9-pentyl-5-(propan-2-yl)-3,6-dihydro-2h-2,6-methano-1-benzoxocin-7-ol
8beta, 11-Dihydroxy-delta(9)-thc
5-Butyl-3-ethenoxy-2-(3-methylcyclohex-2-en-1-yl)phenol
7-Butyl-2-methyl-2-(4-methylpent-3-enyl)chromen-5-ol
(6Ar,7S,10aR)-3,6,6,7,9-pentamethyl-6a,7,8,10a-tetrahydrobenzo[c]chromen-1-ol
[(6Ar,10aR)-1-hydroxy-6,6-dimethyl-3-pentyl-6a,7,10,10a-tetrahydrobenzo[c]chromen-9-yl]methyl2,2-dimethylpropanoate
(17R)-14,18,18-Trimethyl-19-oxatetracyclo[9.8.0.03,9.012,17]nonadeca-1(11),2,9,14-tetraen-10-ol
6,6,9-Trimethyl-3-pentyl-2-prop-2-enyl-6a,7,10,10a-tetrahydrobenzo[c]chromen-1-ol
2,3-Dimethyl-6a,7,8,10a-tetrahydro-6H-benzo[c]chromen-1-ol
(2R,3R)-2-Methyl-2-(4-methylpent-3-enyl)-7-pentyl-3,4-dihydrochromene-3,5-diol
7-(Methoxymethylidene)-6,6-dimethyl-8,9,10,10a-tetrahydro-6aH-benzo[c]chromen-1-ol
6,6,9-Trimethyl-7,8,9,10-tetrahydrobenzo[c]chromen-1-ol
(10Ar)-1-methoxy-6,6,9-trimethyl-3-[(2R)-2-methylbutyl]-6a,7,10,10a-tetrahydrobenzo[c]chromene
4-Cyclohexyl-7-heptan-2-yloxy-2,2-dimethyl-3,4-dihydrochromen-5-ol
6,6,9-Trimethyl-3-pentyl-6a,7,10,10a-tetrahydrobenzo[c]chromene-1,4-diol
4-(7-Methoxy-3,4-dihydro-2H-chromen-4-yl)phenol
4-Cyclohexyl-7-(2,3-dimethylnonan-2-yl)-2,2-dimethylchromen-5-ol
9-Ethyl-6,6-dimethyl-3-(3-methyloctan-2-yl)-7,8,9,10-tetrahydrobenzo[c]chromen-1-ol
6,6-Dimethyl-7,8,9,10-tetrahydrobenzo[c]chromen-1-ol
2-Decan-2-yl-6,6,9-trimethyl-7,8,9,10-tetrahydrobenzo[c]chromen-1-ol
4-Cyclohexyl-2,2-dimethyl-7-(3-methylundecan-2-yl)chromen-5-ol
6,6,9-Trimethyl-3-oct-6-en-2-yl-7,8,9,10-tetrahydrobenzo[c]chromen-1-ol
(6Ar,10aR)-3-(8-bromo-2-methyloctan-2-yl)-9-(methoxymethylidene)-6,6-dimethyl-7,8,10,10a-tetrahydro-6aH-
benzo[c]chromen-1-ol
4-Cyclohex-3-en-1-yl-2,2-dimethyl-7-(3-methyloctan-2-yl)chromen-5-ol
6,6,9-Trimethyl-3-(3-methylundecan-2-yl)benzo[c]chromen-1-ol
4-Cyclopent-2-en-1-yl-2,2-dimethyl-7-octan-2-ylchromen-5-ol
6,6,9-Trimethyl-3-(4-methylnonan-3-yl)benzo[c]chromen-1-ol
6,6,9-Trimethyl-3-(3-methyloct-2-en-2-yl)benzo[c]chromen-1-ol
3-Heptan-2-yloxy-6,6,9-trimethyl-6a,7,8,10a-tetrahydrobenzo[c]chromen-1-ol
8-Hexyl-2,2,3-trimethyl-5-phenyl-3H-1-benzoxepin-6-ol
6,6,9-Trimethyl-3-nonan-3-ylbenzo[c]chromen-1-ol
2,2,8-Trimethyl-5-phenyl-3H-1-benzoxepin-6-ol
3-Butoxy-6,6,9-trimethyl-6a,7,10,10a-tetrahydrobenzo[c]chromen-1-ol
3-Butoxy-6,6,9-trimethyl-6a,7,8,10a-tetrahydrobenzo[c]chromen-1-ol
4-Cyclohexyl-2,2-dimethyl-7-pentylchromen-5-ol
6,6,9-Trimethyl-3-(3-methyloct-2-en-2-yl)-6a,7,8,10a-tetrahydrobenzo[c]chromen-1-ol
4-Cyclohexyl-5-methoxy-2,2-dimethyl-7-(3-methyloctan-2-yl)chromene
6,6,9-Trimethyl-3-octoxy-6a,7,10,10a-tetrahydrobenzo[c]chromen-1-ol
3-(2,3-Dimethylheptan-2-yloxy)-6,6,9-trimethyl-7,8,9,10-tetrahydrobenzo[c]chromen-1-ol
7-Butyl-2,2,4-trimethyl-3,4-dihydrochromen-6-ol
6,6,9-Trimethyl-3-octoxy-6a,7,8,10a-tetrahydrobenzo[c]chromen-1-ol
6,6,10-Trimethyl-3-pentyl-8,9,10,11-tetrahydro-7H-benzo[d][1]benzoxepin-1-ol
3,6,6,10-Tetramethyl-8,9,10,11-tetrahydro-7H-benzo[d][1]benzoxepin-1-ol
6,6,9-Trimethyl-3-(3-methyloctan-2-yl)benzo[c]chromen-1-ol
9-Benzyl-9H-xanthen-1-ol
5-(2-Cyclohexylethenyl)-3-methoxy-2-(3-methylbut-3-enyl)phenol
7-[(6Ar,10aR)-1-hydroxy-6,6,9-trimethyl-6a,7,10,10a-tetrahydrobenzo[c]chromen-3-yl]hept-5-enylnitrate
6,6-Dimethyl-3-pentyl-7,12b-dihydro-6aH-chromeno[3,4-c]chromen-1-ol
2-(4,8-Dimethylnona-3,7-dienyl)-2,5-dimethyl-3,4-dihydrochromen-7-ol
6,6-Dimethylbenzo[c]chromene-1,9-diol
2,2-Dimethyl-7-(2-methyloctan-2-yl)-3,4-dihydrochromen-5-ol
7-Propylspiro[chromene-2,1′-cyclopropane]-5-ol
5-(Cyclopenten-1-yl)-9-methoxy-12-propan-2-ylidene-8-oxatricyclo[7.3.1.02,7]trideca-2,4,6-trien-3-ol
5-Cyclopentyl-9-methoxy-12-propan-2-ylidene-8-oxatricyclo[7.3.1.02,7]trideca-2,4,6-trien-3-ol
5-Hept-5-en-3-yl-9-methoxy-12-propan-2-ylidene-8-oxatricyclo[7.3.1.02,7]trideca-2,4,6-trien-3-ol
12-Propan-2-ylidene-8-oxatricyclo[7.3.1.02,7]trideca-2,4,6-triene-3,9-diol
9-Methoxy-5-(2-methylnonan-2-yl)-12-propan-2-ylidene-8-oxatricyclo[7.3.1.02,7]trideca-2,4,6-trien-3-ol
2,6-Dimethyl-2-(4-methylpentyl)-7-pentylchromen-5-ol
(6R)-6-Ethyl-6,9-dimethyl-3-propyl-6a,7,8,10a-tetrahydrobenzo[c]chromen-1-ol
(6Ar,9R)-6,6,6a,9-tetramethyl-3-pentyl-8,9-dihydro-7H-benzo[c]chromen-1-ol
2,2,4-Trimethyl-3-[(1Z,3Z)-3-methylpenta-1,3-dienyl]-7-propylchromen-5-ol
Cyclohexane; 2,2,7-trimethylchromen-5-ol
Carbanide; 7-cyclopentyl-4-ethenyl-2,2-dimethyl-3-[(Z)-prop-1-enyl]chromen-5-ol; rhenium
2,2-Dimethyl-7-phenyl-3,4-dihydrochromen-5-ol
2,2-Dimethyl-7-(3-propan-2-ylphenyl)-3,4-dihydrochromen-5-ol
2,2-Dimethyl-7-(trifluoromethyl)-3,4-dihydrochromen-5-ol
2,2-Diethyl-7-propan-2-yl-3,4-dihydrochromen-5-ol
7-(3,5-Difluorophenyl)-2,2-dimethyl-3,4-dihydrochromen-5-ol
7-Cyclooctyl-2,2-dimethyl-3,4-dihydrochromen-5-ol
2,2-Diethyl-7-methyl-3,4-dihydrochromen-5-ol
6,9-Dimethyl-3-pentyl-6H-benzo[c]chromen-1-ol
7-(5-Fluoropentyl)-2-methyl-2-(4-methylpent-3-enyl)chromen-5-ol
(6Ar)-3-[(E)-hept-1-enyl]-9-methyl-6-methylidene-6a,7,8,10a-tetrahydrobenzo[c]chromen-1-ol
8-[(6Ar)-1-hydroxy-9-methyl-6-methylidene-6a,7,8,10a-tetrahydrobenzo[c]chromen-3-yl]octanoic acid
(6Ar)-3-but-3-ynyl-9-methyl-6-methylidene-6a,7,8,10a-tetrahydrobenzo[c]chromen-1-ol
5-[(6Ar,10aR)-1-hydroxy-9-methyl-6-methylidene-6a,7,8,10a-tetrahydrobenzo[c]chromen-3-yl]pentanenitrile
(6Ar,10aR)-3-(4-hydroxybutyl)-9-methyl-6-methylidene-6a,7,8,10a-tetrahydrobenzo[c]chromen-1-ol
(6Ar,10aR)-9-methyl-3-(3-methylhexan-2-yl)-6-methylidene-6a,7,8,10a-tetrahydrobenzo[c]chromen-1-ol
(6Ar)-3-(4-chlorobutyl)-9-methyl-6-methylidene-6a,7,8,10a-tetrahydrobenzo[c]chromen-1-ol
(6Ar)-9-methyl-6-methylidene-3-[(E)-pent-3-enyl]-6a,7,8,10a-tetrahydrobenzo[c]chromen-1-ol
(6Ar,10aR)-9-methyl-6-methylidene-3-tridecyl-6a,7,8,10a-tetrahydrobenzo[c]chromen-1-ol
(6Ar)-9-methyl-6-methylidene-3-pent-4-ynyl-6a,7,8,10a-tetrahydrobenzo[c]chromen-1-ol
7-[(6Ar)-1-hydroxy-9-methyl-6-methylidene-6a,7,8,10a-tetrahydrobenzo[c]chromen-3-yl]heptanoic acid
(6Ar)-9-methyl-6-methylidene-3-(4-methylpentyl)-6a,7,8,10a-tetrahydrobenzo[c]chromen-1-ol
(6Ar)-3-hept-5-ynyl-9-methyl-6-methylidene-6a,7,8,10a-tetrahydrobenzo[c]chromen-1-ol
(6Ar)-9-methyl-6-methylidene-3-propan-2-yl-6a,7,8,10a-tetrahydrobenzo[c]chromen-1-ol
(6Ar)-9-methyl-3-(3-methylhexan-2-yl)-6-methylidene-6a,7,8,10a-tetrahydrobenzo[c]chromen-1-ol
(6Ar)-9-methyl-6-methylidene-3-(3-methylpentyl)-6a,7,8,10a-tetrahydrobenzo[c]chromen-1-ol
(6Ar)-9-methyl-6-methylidene-3-[(E)-oct-1-enyl]-6a,7,8,10a-tetrahydrobenzo[c]chromen-1-ol
(6Ar)-9-methyl-6-methylidene-3-[(E)-pent-2-enyl]-6a,7,8,10a-tetrahydrobenzo[c]chromen-1-ol
(6Ar)-9-methyl-6-methylidene-3-(2-methylpentyl)-6a,7,8,10a-tetrahydrobenzo[c]chromen-1-ol
(6Ar)-3-[(E)-but-1-enyl]-9-methyl-6-methylidene-6a,7,8,10a-tetrahydrobenzo[c]chromen-1-ol
(6Ar,10aR)-9-methyl-6-methylidene-3-(4-methylsulfanylbutyl)-6a,7,8,10a-tetrahydrobenzo[c]chromen-1-ol
(6Ar,10aR)-3-(4-aminobutyl)-9-methyl-6-methylidene-6a,7,8,10a-tetrahydrobenzo[c]chromen-1-ol
(6Ar)-9-methyl-6-methylidene-3-(3-phenylpropyl)-6a,7,8,10a-tetrahydrobenzo[c]chromen-1-ol
(E)-3-[(6Ar)-1-hydroxy-9-methyl-6-methylidene-6a,7,8,10a-tetrahydrobenzo[c]chromen-3-yl]prop-2-enoic acid
(6Ar)-9-methyl-6-methylidene-3-octyl-6a,7,8,10a-tetrahydrobenzo[c]chromen-1-ol
(6Ar)-3-hexan-2-yl-9-methyl-6-methylidene-6a,7,8,10a-tetrahydrobenzo[c]chromen-1-ol
(6Ar)-9-methyl-6-methylidene-3-[(E)-pent-1-enyl]-6a,7,8,10a-tetrahydrobenzo[c]chromen-1-ol
(Z)-3-[(6Ar)-1-hydroxy-9-methyl-6-methylidene-6a,7,8,10a-tetrahydrobenzo[c]chromen-3-yl]prop-2-enoic acid
(10Ar)-3-butyl-6,6,9-trimethyl-6a,7,8,10a-tetrahydrobenzo[c]chromen-1-ol
Ethane; 3-ethyl-6,6,9-trimethylbenzo[c]chromen-1-ol
5-Ethyl-2-(3-methylcyclohex-2-en-1-yl)-3-propan-2-yloxyphenol
(6Ar,10aR)-6,6,9-trimethyl-3-pentyl-6a,7,8,10a-tetrahydrobenzo[c]chromen-1-ol; acetic acid; ethane
(10Ar)-6,6,9-trimethyl-3-(2-methylpentan-2-yl)-6a,7,10,10a-tetrahydrobenzo[c]chromen-1-ol
(6Ar)-6,6,9-trimethyl-3-(2-methylhexan-2-yl)-6a,7,10,10a-tetrahydrobenzo[c]chromen-1-ol; ethane; phosphane; vanadium
3-(8-Hydroxy-2-methyloctan-2-yl)-6,6,9-trimethyl-6a,7,10,10a-tetrahydrobenzo[c]chromen-1-ol
(10Ar)-3-(8-hydroxy-2-methyloctan-2-yl)-6,6,9-trimethyl-6a,7,10,10a-tetrahydrobenzo[c]chromen-1-ol
(10Ar)-6,6,9-trimethyl-3-(2-methylhexan-2-yl)-6a,7,10,10a-tetrahydrobenzo[c]chromen-1-ol
(10Ar)-6,6,9-trimethyl-3-(2-methylhexan-2-yl)-6a,7,10,10a-tetrahydrobenzo[c]chromen-1-ol; ethane; phosphane; vanadium
(6Ar,10aR)-6,6,9-trimethyl-3-(2-methylpentan-2-yl)-6a,7,10,10a-tetrahydrobenzo[c]chromen-1-ol; ethane; phosphane; vanadium
(6Ar,10aR)-1-methoxy-6,6-dimethyl-3-(2-methylpentan-2-yl)-9-(trifluoromethyl)-6a,7,10,10a-tetrahydrobenzo[c]chromene
9-(Hydroxymethyl)-6,6-dimethyl-3-propyl-6a,7,10,10a-tetrahydrobenzo[c]chromen-1-ol
(6Ar)-9-chloro-6,6-dimethyl-3-propyl-6a,7,8,10a-tetrahydrobenzo[c]chromen-1-ol
3-Hexyl-6,6,9-trimethylbenzo[c]chromen-1-ol
(6Ar,10aR)-3-(2-hexylthian-2-yl)-6,6,9-trimethyl-6a,7,10,10a-tetrahydrobenzo[c]chromen-1-ol
(6Ar)-3-[1-(2-methoxyethoxy)ethyl]-6,6,9-trimethyl-6a,7,8,10a-tetrahydrobenzo[c]chromen-1-ol
5-[2-(2-Hydroxyphenyl)ethyl]-2-methyl-3-(2-methylbut-3-en-2-yloxy)phenol
2-[(4R)-4-Methyl-3,4-dihydro-2H-chromen-7-yl]phenol
(2Ar,3S,8bR)-2-ethyl-1,1,3-trimethyl-6-pentyl-2,2a,3,8b-tetrahydrocyclobuta[c]chromen-8-ol
(10Ar)-3,6,6,9-tetramethyl-6a,7,8,9,10,10a-hexahydrobenzo[c]chromen-1-ol
(6Ar,10aR)-9-methyl-6-methylidene-3-undecyl-6a,7,8,10a-tetrahydrobenzo[c]chromen-1-ol
7-Propyl-2H-chromen-5-ol
(6Ar,10aR)-3-(4-chlorobutyl)-9-methyl-6-methylidene-6a,7,8,10a-tetrahydrobenzo[c]chromen-1-ol
(1R,9R,12S)-9-Methyl-12-propan-2-yl-5-propyl-8-oxatricyclo[7.3.1.02,7]trideca-2,4,6-trien-3-ol
(6Ar,10aR)-9-methyl-6-methylidene-3-nonyl-6a,7,8,10a-tetrahydrobenzo[c]chromen-1-ol
(6Ar,10aR)-3-decyl-9-methyl-6-methylidene-6a,7,8,10a-tetrahydrobenzo[c]chromen-1-ol
(6Ar,10aR)-9-methyl-6-methylidene-3-octyl-6a,7,8,10a-tetrahydrobenzo[c]chromen-1-ol
(2S)-2-Methyl-7-propyl-3,4-dihydro-2H-chromen-5-ol
(6Ar,10aR)-9-methyl-6-methylidene-3-propan-2-yl-6a,7,8,10a-tetrahydrobenzo[c]chromen-1-ol
2-Ethyl-2-phenyl-3,4-dihydrochromen-7-ol
3,3-Dimethyl-11-(2-methyloctan-2-yl)-2,8-dioxatricyclo[7.3.1.05,13]trideca-1(12),9(13),10-triene
3-(2,4-Dimethoxyphenyl)-9H-xanthen-1-ol
7-Propan-2-ylspiro[chromene-2,1′-cyclobutane]-5-ol
6,6-Diethyl-9-methyl-3-pentylbenzo[c]chromen-1-ol
9-Cyclobutyl-5,5,7,7-tetramethyl-8,9-dihydro-6H-xanthene-1,3-diol
2-(4,8-Dimethylnona-3,7-dienyl)-2-methyl-7-pentylchromen-5-ol
6,6,9-Trimethyl-3-pent-1-enylbenzo[c]chromen-1-ol
9-Azido-6,6-dimethyl-3-pentyl-6a,7,8,10a-tetrahydrobenzo[c]chromen-1-ol
3-(2-Methoxyphenyl)-9H-xanthen-1-ol
7-Fluoro-2,2-dimethyl-3,4-dihydrochromen-5-ol
(6Ar,10aR)-3-(8-hydroxy-2-methyloctan-2-yl)-6,6,9-trimethyl-6a,7,10,10a-tetrahydrobenzo[c]chromen-1-ol
3-(2,4-Dimethoxyphenyl)-9H-xanthene-1,8-diol
3-(2-Hydroxyphenyl)-9H-xanthen-1-ol
6,6-Dimethyl-3-(2-methyloctan-2-yl)-7,8,9,10,11,11a-hexahydro-6aH-cyclohepta[c]chromen-1-ol
7-Ethyl-8-methyl-7,8,9,10-tetrahydro-6H-benzo[c]chromen-3-ol
9-Azido-6,6-dimethyl-3-pentyl-6a,7,10,10a-tetrahydrobenzo[c]chromen-1-ol
1-Azidooxy-6,6,9-trimethyl-3-pentyl-6a,7,10,10a-tetrahydrobenzo[c]chromene
6,6,8-Trimethyl-7,8,9,10-tetrahydrobenzo[c]chromen-1-ol
(6Ar,10aR)-1-hydroxy-6,6-dimethyl-3-(2-methyloctan-2-yl)-6a,7,10,10a-tetrahydrobenzo[c]chromene-9-carbonitrile
3-Decoxy-2-(3-methylbutyl)-5-[(E)-2-phenylethenyl]phenol
3-Pentyl-7,8,9,10-tetrahydro-6H-benzo[c]chromen-1-ol
4-Tert-butyl-2,2-dimethyl-6-propan-2-yl-3,4-dihydrochromen-5-ol
2-Methyl-7-pentyl-2-(4,8,12-trimethyltrideca-3,7,11-trienyl)chromen-5-ol
7-Propan-2-ylspiro[chromene-2,1′-cyclopropane]-5-ol
7-Cyclopentylspiro[chromene-2,1′-cyclobutane]-5-ol
(6As,10aS)-3-(7-iodo-2-methylheptan-2-yl)-6,9-dimethylidene-7,8,10,10a-tetrahydro-6aH-benzo[c]chromen-1-ol
(6Ar,10aR)-6,6,9,10a-tetramethyl-3-pentyl-7,10-dihydro-6aH-benzo[c]chromen-1-ol
2-(3-Hydroxy-4-methoxycyclohexen-1-yl)-5-methoxy-7-methyl-3,4-dihydro-2H-chromen-4-ol
2-[(3R)-8,8-Dimethyl-3,4,9,10-tetrahydro-2H-pyrano[2,3-h]chromen-3-yl]-5-methylphenol
(6Ar,10aS)-6,6,6a,9-tetramethyl-3-pentyl-8,9,10,10a-tetrahydro-7H-benzo[c]chromen-1-ol
(2R)-2-Methyl-2,7-dipropyl-3,4-dihydrochromen-5-ol
5-(3-Methoxypropoxy)-7-propyl-3,4-dihydro-2H-chromene
3-[1-(2,2-Dimethyl-4,5-dihydro-3H-chromen-5-id-6-yl)propan-2-yl]-5-methoxyphenol; uranium
(6As)-1-hydroxy-6,6-dimethyl-3-pentyl-7,8,10,10a-tetrahydro-6aH-benzo[c]chromen-9-one
8-[(6Ar,10aR)-1-hydroxy-9-methyl-6-methylidene-6a,7,8,10a-tetrahydrobenzo[c]chromen-3-yl]octanoic acid
5-[1-(2-Hydroxy-6-methoxyphenyl)ethyl]-2-(2-hydroxypropan-2-yl)-3-methoxyphenol
(6Ar,10aR)-3-hexan-2-yl-9-methyl-6-methylidene-6a,7,8,10a-tetrahydrobenzo[c]chromen-1-ol
6,6-Difluoro-3-(2-methylbutan-2-yl)-6a,7,8,9,10,10a-hexahydrobenzo[c]chromen-1-ol
(6Ar)-6,6,9-trimethyl-3-pentyl-6a,7,8,10a-tetrahydrobenzo[c]chromene-1,2-diol
(10S)-10-Ethoxy-6,6,9,9-tetramethyl-3-pentyl-8,10-dihydro-7H-benzo[c]chromen-1-ol
(6As,10aS)-6a-iodo-6,9-dimethyl-3-pentyl-6,7,8,10a-tetrahydrobenzo[c]chromen-1-ol
(6Ar,10aR)-3-(4-iodobutyl)-6,6,6a,9-tetramethyl-8,10a-dihydro-7H-benzo[c]chromen-1-ol
6a,7,8,10a-Tetrahydro-6H-benzo[c]chromen-1-ol
(6Ar,10aR)-9-methyl-6-methylidene-3-(5-phenylpentyl)-6a,7,8,10a-tetrahydrobenzo[c]chromen-1-ol
(6Ar,10aR)-9-methyl-6-methylidene-3-[(E)-pent-1-enyl]-6a,7,8,10a-tetrahydrobenzo[c]chromen-1-ol
(10As)-3-tert-butyl-6,6,9-trimethyl-7,10,10a,11-tetrahydro-6aH-benzo[c][1]benzoxepin-1-ol
5-Butan-2-yl-3-ethoxy-2-methylphenol;1,3-dimethyl-5-(3-methylbutan-2-yl)benzene-2-ide; uranium(2+)
6,6-Difluoro-3-(3-methylbutan-2-yl)-6a,7,8,9,10,10a-hexahydrobenzo[c]chromen-1-ol
9-But-3-en-2-ylxanthene-3,6,9-triol
(10Ar)-9-(hydroxymethyl)-6,6-dimethyl-3-pentyl-6a,7,8,9,10,10a-hexahydrobenzo[c]chromen-1-ol
1-Methoxy-3,6,6,9-tetramethyl-6a,7,8,10a-tetrahydrobenzo[c]chromene
(1R,9S)-9,11,11-Trimethyl-5-(2-methyloctan-2-yl)-8-oxatricyclo[7.4.1.02,7]tetradeca-2,4,6-trien-3-ol
(6As,10aR)-6a-ethenyl-3-(4-iodobutyl)-6,6,9,10a-tetramethyl-7,8-dihydrobenzo[c]chromen-1-ol
3-(4-Hydroxyphenyl)-5-methyl-3,4-dihydro-2H-chromen-7-ol
(3Ar)-9-methoxy-2,3a,4,4-tetramethyl-7-pentyl-3,9b-dihydrocyclopenta[c]chromene
7-(4-lodobutyl)-2-methyl-2-(4-methylpent-3-enyl)chromen-5-ol
6,9-Dimethyl-3-pentyl-7H-benzo[c]chromen-1-ol
4,6,8-Trimethyl-9H-xanthen-3-ol
(6Ar,10aR)-9-methyl-6-methylidene-3-(6-phenylhexyl)-6a,7,8,10a-tetrahydrobenzo[c]chromen-1-ol
(Z)-3-[(6Ar,10aR)-1-hydroxy-9-methyl-6-methylidene-6a,7,8,10a-tetrahydrobenzo[c]chromen-3-yl]prop-2-enoic acid
(E)-3-[(6Ar,10aR)-1-hydroxy-9-methyl-6-methylidene-6a,7,8,10a-tetrahydrobenzo[c]chromen-3-yl]prop-2-enoic acid
(6Ar,10aR)-9-methyl-6-methylidene-3-(4-phenylbutyl)-6a,7,8,10a-tetrahydrobenzo[c]chromen-1-ol
1-Ethyl-8-methyl-9H-xanthene-3,6-diol
(6Ar,10aR)-3-hexyl-9-methyl-6-methylidene-6a,7,8,10a-tetrahydrobenzo[c]chromen-1-ol
(6Ar,10aR)-3-[(E)-hept-1-enyl]-9-methyl-6-methylidene-6a,7,8,10a-tetrahydrobenzo[c]chromen-1-ol
(6Ar,10aR)-9-methyl-6-methylidene-3-[(E)-pent-2-enyl]-6a,7,8,10a-tetrahydrobenzo[c]chromen-1-ol
(6Ar,10aS)-9-iodo-6,6-dimethyl-3-pentyl-6a,7,8,10a-tetrahydrobenzo[c]chromen-1-ol
(6As,10aS)-3,6,6,9-tetramethyl-6a,7,10,10a-tetrahydrobenzo[c]chromen-1-ol
3-[(4R)-4-Ethyl-5-hydroxy-2,2-dimethyl-7-pentyl-3,4-dihydrochromen-3-yl]propanenitrile
(6S,6Ar,10aR)-6-ethynyl-9-(hydroxymethyl)-6-methyl-3-pentyl-6a,7,10,10a-tetrahydrobenzo[c]chromen-1-ol
(6As,10aS)-3-[4-[(Z)-hex-1-enyl]oxan-4-yl]-9-(hydroxymethyl)-6-methylidene-6a,7,8,10a-tetrahydrobenzo[c]chromen-1-ol
(6Ar,10aR)-9-methyl-6-methylidene-3-(2-methylpentyl)-6a,7,8,10a-tetrahydrobenzo[c]chromen-1-ol
(6Ar,10aR)-3-hex-5-ynyl-9-methyl-6-methylidene-6a,7,8,10a-tetrahydrobenzo[c]chromen-1-ol
(2S)-2-Methyl-7-pentyl-3,4-dihydro-2H-chromen-5-ol
(6Ar,10aR)-9-methyl-6-methylidene-3-(3-phenylpropyl)-6a,7,8,10a-tetrahydrobenzo[c]chromen-1-ol
(6Ar,10aR)-9-methyl-6-methylidene-3-pent-4-ynyl-6a,7,8,10a-tetrahydrobenzo[c]chromen-1-ol
9-Cyclopropyl-6,6-dimethyl-3-pentyl-6a,7,8,10a-tetrahydrobenzo[c]chromen-1-ol
4a,9,9-Trimethyl-4H-xanthene-3,6-diol
3-Butyl-6,6-dimethyl-10,10a-dihydrobenzo[c]chromen-1-ol
3-(4-Methoxy-5-methyl-3,6-dimethylidenecyclohexa-1,4-dien-1-yl)-3,4-dihydro-2H-chromen-7-ol
3,6,6,9-Tetramethyl-7,8-dihydrobenzo[c]chromen-1-ol
(6Ar,10aR)-9-methyl-6-methylidene-3-(4-methylpentyl)-6a,7,8,10a-tetrahydrobenzo[c]chromen-1-ol
2-(3-Methylcyclohex-2-en-1-yl)-3-prop-1-en-2-yloxy-5-propylphenol
2-(8-Butyl-7-ethoxy-3,4-dihydro-2H-chromen-3-yl)-5-methylphenol
1-Methoxy-6,6,9-trimethyl-3-propyl-6a,7,8,9,10,10a-hexahydrobenzo[c]chromene
[3-Hydroxy-2-[(1S)-3-methylcyclohex-2-en-1-yl]-5-propylphenyl]acetate
3-Methoxy-2-[(1S)-3-methylcyclohex-2-en-1-yl]-5-pentylphenol
4-[(3R,4S)-3-Cyclohexa-2,4-dien-1-yl-7-methoxy-2,2-dimethyl-3,4-dihydrochromen-4-yl]cyclohexa-1,5-dien-1-ol
(6Ar,10aR)-6,6,6a,9-tetramethyl-3-propyl-8,10a-dihydro-7H-benzo[c]chromen-1-ol
(6As,10aS)-3-(1-hexylcyclopropyl)-9-(hydroxymethyl)-6-methylidene-6a,7,8,10a-tetrahydrobenzo[c]chromen-1-ol
CID 148030134
(3S)-3-(4-Methoxy-2-methylphenyl)-3,4-dihydro-2H-chromen-7-ol
(6Ar,10aR)-9-methyl-6-methylidene-3-[(E)-oct-1-enyl]-6a,7,8,10a-tetrahydrobenzo[c]chromen-1-ol
3-Methoxy-2-[(1R)-3-methyl-6-prop-1-en-2-ylcyclohex-2-en-1-yl]-5-pentylphenol
(6Ar,10aR)-6,6-dimethyl-3-pentyl-10a-prop-1-en-2-yl-7,8,9,10-tetrahydro-6aH-benzo[c]chromen-1-ol
(1R,9R)-9,12-Dimethyl-5-pentyl-12-prop-1-en-2-yl-8-oxatricyclo[7.3.1.02,7]trideca-2,4,6-trien-3-ol
6,6-Difluoro-1-methoxy-9-methyl-3-(3-methylhex-5-en-2-yl)-6a,7,8,10a-tetrahydrobenzo[c]chromene
(6Ar,10aR)-3-(4-chlorobutyl)-6,9-dimethyl-6a,7,8,10a-tetrahydro-6H-benzo[c]chromen-1-ol
(1R,9R,12S)-9,12,13,13-Tetramethyl-5-pentyl-8-oxatricyclo[7.4.1.02,7]tetradeca-2,4,6-trien-3-ol
2,3-Dimethyl-6-[7-(2-methylbutan-2-yloxy)-3,4-dihydro-2H-chromen-3-yl]phenol
6-Ethyl-4′-propylspiro[3H-1-benzofuran-2,1′-cyclohexane]-4-ol
3-(3-Methoxypropoxy)-6,6,9-trimethyl-6a,7,8,10a-tetrahydrobenzo[c]chromen-1-ol
(10Ar)-1-methoxy-6,6,9-trimethyl-3-pentyl-6a,7,8,10a-tetrahydrobenzo[c]chromene
(6As,10aS)-3-(1-adamantyl)-6,9-dimethylidene-7,8,10,10a-tetrahydro-6aH-benzo[c]chromen-1-ol
7-(2-lodoethyl)-2-methyl-2-(4-methylpent-3-enyl)chromen-5-ol
3-Propan-2-yl-5-(2-propan-2-ylphenoxy)phenol
(6As,10aS)-3-ethyl-6,6,9-trimethyl-6a,7,10,10a-tetrahydrobenzo[c]chromen-1-ol
(6Ar)-3-ethyl-6,6,9-trimethyl-6a,7,10,10a-tetrahydrobenzo[c]chromen-1-ol
3-Butyl-6,6-dimethylbenzo[c]chromen-1-ol
(6Ar,10aR)-3-but-3-ynyl-9-methyl-6-methylidene-6a,7,8,10a-tetrahydrobenzo[c]chromen-1-ol
(1R,9R,12R)-9,12-Dimethyl-12-prop-1-en-2-yl-5-propyl-8-oxatricyclo[7.3.1.02,7]trideca-2,4,6-trien-3-ol
(3R)-3,4-Diethyl-2,2-dimethyl-7-propyl-3,4-dihydrochromen-5-ol
(E)-6-[(10Ar)-1-hydroxy-6,6,9-trimethyl-6a,7,10,10a-tetrahydrobenzo[c]chromen-3-yl]-6-methylhept-2-enenitrile
(3S)-8-Methyl-4-methylidene-3-(4-methylphenyl)-2,3-dihydrochromen-7-ol
(6Ar,9R,10aR)-3-(1-butoxy-2-methylpropan-2-yl)-6,6-dimethyl-6a,7,8,9,10,10a-hexahydrobenzo[c]chromene-1,9-diol
(6Ar,10aR)-3-(2-iodoethyl)-6,6,9-trimethyl-6a,7,8,10a-tetrahydrobenzo[c]chromen-1-ol
(6Ar,10aR)-3-[(E)-but-1-enyl]-9-methyl-6-methylidene-6a,7,8,10a-tetrahydrobenzo[c]chromen-1-ol
(6Ar,10aR)-9-methyl-6-methylidene-3-(3-methylpentyl)-6a,7,8,10a-tetrahydrobenzo[c]chromen-1-ol
6,9-Dimethyl-3-propyl-10aH-benzo[c]chromen-1-ol
(10Ar)-6-[2-[(6aR,10aR)-1-hydroxy-6,6-dimethyl-3-pentyl-6a,7,8,10a-tetrahydrobenzo[c]chromen-9-yl]ethyl]-6,6a,9,10-
tetramethyl-3-propyl-8,9,10,10a-tetrahydro-7H-benzo[c]chromen-1-ol
3-(2-lodoethyl)-6,6,9-trimethylbenzo[c]chromen-1-ol
CID 146880247
(6Ar,10aR)-9-methyl-6-methylidene-3-[(E)-pent-3-enyl]-6a,7,8,10a-tetrahydrobenzo[c]chromen-1-ol
2-[2-(5-Methoxy-2,2-dimethyl-3,4-dihydrochromen-6-yl)ethyl]-4,5-dimethylphenol
(1R,9S)-9,11,11-Trimethyl-5-(3-methylnonan-3-yl)-8-oxatricyclo[7.4.1.02,7]tetradeca-2,4,6-trien-3-ol
2-Methyl-7-pentyl-2-[(3E,7E)-4,8,12-trimethyltrideca-3,7,11-trienyl]chromen-5-ol
2-[(3E)-4,8-Dimethylnona-3,7-dienyl]-2-methyl-7-pentylchromen-5-ol
2-Methyl-7-(2-methyloctan-2-yl)-2-(4-methylpent-3-enyl)chromen-5-ol
(3Ar,9aS)-1-(2-iodopropan-2-yl)-3a-methyl-6-pentyl-2,3,9,9a-tetrahydro-1H-cyclopenta[b]chromen-8-ol
(6Ar,9R)-6,6,9-trimethyl-3-pentyl-6a,7,8,9-tetrahydrobenzo[c]chromen-1-ol
(10As)-9-(fluoromethyl)-6-methyl-3-(2-methyloctan-2-yl)-6a,7,8,10a-tetrahydro-6H-benzo[c]chromen-1-ol
3-Ethoxy-2-[3-(fluoromethyl)cyclohex-2-en-1-yl]-5-(2-methyloctan-2-yl)phenol
[(6Ar,10aR)-1-hydroxy-3,6,6,9-tetramethyl-6a,7,8,10a-tetrahydrobenzo[c]chromen-4-yl]methanediol
5-(8,8-Dimethyl-3,4,9,10-tetrahydro-2H-pyrano[2,3-h]chromen-3-yl)-2-ethylphenol
7,7,10-Trimethyl-3-pentyl-7a,8,9,11a-tetrahydro-6H-benzo[d][1]benzoxepin-1-ol
(6Z)-2,2,6-Trimethyl-11-pentyl-3,4,5,8-tetrahydro-1-benzoxecin-9-ol
2-(3-Methyl-6-prop-1-en-2-ylcyclohex-2-en-1-yl)-5-pentyl-3-(2-propan-2-yloxyethoxy)phenol
(1S,9R)-9,13,13-Trimethyl-5-pentyl-8-oxatetracyclo[7.4.1.02,7.012,14]tetradeca-2,4,6,12(14)-tetraen-3-ol
9-Hydroxyimino-6,6-dimethyl-3-pentyl-7,8,10,10a-tetrahydro-6aH-benzo[c]chromen-1-ol
7-(Cyclopenten-1-yl)-2-ethoxy-4-(2-methylpent-2-en-3-yl)-3,4-dihydro-2H-chromen-5-ol
7-(3-Methyloctan-2-yl)-4-(2-methylpent-2-en-3-yl)-3,4-dihydro-2H-chromene-2,5-diol
7-Heptyl-4-(2-methylpent-2-en-3-yl)-3,4-dihydro-2H-chromene-2,5-diol
7-(Cyclohepten-1-yl)-4-(2-methylpent-2-en-3-yl)-3,4-dihydro-2H-chromene-2,5-diol
2-Methyl-4-(2-methylpent-1-en-3-yl)-7-pentyl-3,4-dihydro-2H-chromen-5-ol
2-Methoxy-7-[(E)-3-methyloct-2-en-2-yl]-4-(2-methylpent-2-en-3-yl)-3,4-dihydro-2H-chromen-5-ol
7-Cyclohexyl-4-(2-methylpent-2-en-3-yl)-3,4-dihydro-2H-chromene-2,5-diol
7-Decyl-2-methoxy-4-(2-methylpent-2-en-3-yl)-3,4-dihydro-2H-chromen-5-ol
4-(2-Methylpent-2-en-3-yl)-7-pentyl-3,4-dihydro-2H-chromene-2,5-diol
7-[(E)-4-Methylhex-4-en-3-yl]-4-(2-methylpent-2-en-3-yl)-3,4-dihydro-2H-chromene-2,5-diol
2-Methyl-4-(2-methylpent-2-en-3-yl)-7-pentyl-3,4-dihydro-2H-chromen-5-ol
7-Decyl-4-(2-methylpent-2-en-3-yl)-3,4-dihydro-2H-chromene-2,5-diol
7-[(E)-Hex-2-enyl]-4-(2-methylpent-2-en-3-yl)-3,4-dihydro-2H-chromene-2,5-diol
9-Methyl-9,9a-dihydro-1H-xanthene-3,6-diol
Ethane; 2,4,7-trimethyl-9-propan-2-yl-9H-xanthene-3,6-diol
9-(Hydroxymethyl)-6,6-dimethyl-3-(2-methyloctan-2-yl)-6a,7,10,10a-tetrahydrobenzo[c]chromen-1-ol; molecular hydrogen
(3S)-3-[(E)-3-(Hydroxymethyl)pent-2-enyl]-2,2-dimethyl-7-(2-methyloctan-2-yl)-3,4-dihydrochromen-5-ol
(6As,10aS)-9-(hydroxymethyl)-6,6-dimethyl-3-(2-methyloctan-2-yl)-6a,7,10,10a-tetrahydrobenzo[c]chromen-1-olate
2-[(3S)-8,8-Dimethyl-3,4,9,10-tetrahydro-2H-pyrano[2,3-h]chromen-3-yl]-5-methylphenol
Ethane; 2-[(6,6,9-trimethyl-3-propyl-6a,7,8,10a-tetrahydrobenzo[c]chromen-1-yl)oxy]prop-2-en-1-ol
3-[(6,6,9-Trimethyl-3-propyl-6a,7,8,10a-tetrahydrobenzo[c]chromen-1-yl)oxy]but-3-en-1-ol
6,6,9-Trimethyl-3-pentyl-7,8,9,10-tetrahydrobenzo[c]chromene-1,10-diol
6,6-Dimethyl-3-pentyl-7,8,9,10-tetrahydrobenzo[c]chromene-1,9-diol; ethane
Ethane; 6,6,9-trimethyl-3-pentyl-6a,7,8,10a-tetrahydrobenzo[c]chromen-1-ol
Ethane; 2-(3-methylcyclohex-2-en-1-yl)-3-propan-2-yloxy-5-propylphenol
5-Methyl-2-(3-methylcyclohex-2-en-1-yl)-3-propan-2-yloxyphenol
3-Methyl-5-[2-(4-methylcyclohex-3-en-1-yl)propan-2-yloxy]phenol
2,5-Dicyclopropyl-3-[(2-methylpropan-2-yl)oxy]phenol
(6Ar)-6-(3-cyclopropylbutyl)-6,6a,9-trimethyl-3-pentyl-10,10a-dihydro-7H-benzo[c]chromen-1-ol
3-[(3S)-3-Methylhexan-3-yl]oxy-2-prop-1-en-2-yl-5-propylphenol
(6Ar,10aR)-9-methyl-6-methylidene-3-(2-methyloctan-2-yl)-6a,7,8,10a-tetrahydrobenzo[c]chromen-1-ol
5-Butyl-2-(3-methylcyclohex-2-en-1-yl)-3-propan-2-yloxyphenol
9-[(3Z)-Penta-1,3-dien-2-yl]xanthene-3,6,9-triol
2-(3-Methylcyclohex-3-en-1-yl)-5-(2-methylhexan-2-yl)-3-propan-2-yloxyphenol
(6As)-3-(8-bromo-2-methyloctan-2-yl)-6,6-dimethyl-6a,7,10,10a-tetrahydrobenzo[c]chromene-1,9-diol
(6Ar,10aR)-3-heptyl-9-(hydroxymethyl)-6,6-dimethyl-6a,7,8,9,10,10a-hexahydrobenzo[c]chromen-1-ol
Ethane; (4E)-2,2,3-trimethyl-4-(2-methylpropylidene)-7-pentyl-3H-chromen-5-ol
Ethane; 3-[1-(4-methylcyclohex-3-en-1-yl)ethenoxy]-5-pentylphenol
Ethane; 2-(3-methylcyclohex-3-en-1-yl)-5-oct-1-en-2-yl-3-propan-2-yloxyphenol
5-(3-Hydroxynonan-3-yl)-2-(3-methylcyclohex-3-en-1-yl)-3-propan-2-yloxyphenol
5-(3-Hydroxy-2-methylbut-3-en-2-yl)-2-(3-methylcyclohex-3-en-1-yl)-3-propan-2-yloxyphenol
3,5-Dimethyl-3,4-dihydro-2H-chromen-7-ol
Ethane; (3S,4S)-2,3,4-trimethyl-3,4-dihydro-2H-chromene-5,7-diol
(10Ar)-6,6,9-trimethyl-3-pentyl-6a,7,10,10a-tetrahydrobenzo[c]chromen-1-ol; molecularhydrogen
Ethane; (6,6,9-trimethyl-3-pentyl-6a,7,8,10a-tetrahydrobenzo[c]chromen-1-yl)oxyphosphinousacid
(6Ar)-6,6,9-trimethyl-3-pentyl-6a,7,8,10a-tetrahydrobenzo[c]chromen-1-ol; ethane; propane; prop-1-ene; uranium; hydrate
2-Methyl-3-methylidenehex-1-ene; 6,6,9-trimethyl-3-pentyl-6a,7,8,10a-tetrahydrobenzo[c]chromen-1-ol
Ethane; 2-methylbut-1-ene; 6,6,9-trimethyl-3-pentyl-6a,7,8,10a-tetrahydrobenzo[c]chromen-1-ol
(3R)-3-Butyl-2,2,4-trimethyl-7-pentyl-3,4-dihydrochromen-5-ol; 2-methylbuta-1,3-diene;propane; propan-1-ol; trihydrate
3-Ethenoxy-2-(3-methylcyclohex-2-en-1-yl)-5-pentylphenol
Ethane; 5-[(E)-2-(4-hydroxyphenyl)ethenyl]-2,2-dimethyl-3,4-dihydrochromen-7-ol
3-(2-Butyl-7-methoxy-2-propyl-3,4-dihydrochromen-4-yl)phenol
6,6-Difluoro-9-methyl-3-(3-methylbutan-2-yl)-6a,7,8,10a-tetrahydrobenzo[c]chromen-1-ol
6,6-Difluoro-3,9-dimethyl-6a,7,10,10a-tetrahydrobenzo[c]chromen-1-ol
6,6-Difluoro-9-methyl-3-(2-methylbutan-2-yl)-6a,7,10,10a-tetrahydrobenzo[c]chromen-1-ol
6,6-Difluoro-9-methyl-3-(3-methylbutan-2-yl)-6a,7,8,9,10,10a-hexahydrobenzo[c]chromen-1-ol
(6Ar,10aS)-6-fluoro-3-methyl-6-phosphanyl-6a,7,8,9,10,10a-hexahydrobenzo[c]chromen-1-ol;ethane
6,6-Difluoro-9-methyl-3-(2-methylbutan-2-yl)-6a,7,8,10a-tetrahydrobenzo[c]chromen-1-ol
6,6-Difluoro-9-methyl-3-(2-methylbutan-2-yl)-6a,7,8,9,10,10a-hexahydrobenzo[c]chromen-1-ol
6,6-Difluoro-3,9-dimethyl-6a,7,8,10a-tetrahydrobenzo[c]chromen-1-ol
5-Butoxy-7-ethyl-3,4-dihydro-2H-chromene; ethane
(11Ar)-6,6,10-trimethyl-3-pentyl-7,8,9,11a-tetrahydro-6aH-cyclohepta[c]chromen-1-ol
(6Ar)-6,6,9-trimethyl-3-pentyl-6a,7,8,10a-tetrahydrobenzo[c]chromen-1-ol; ethane
Ethane; 3-methoxy-5-methyl-2-propan-2-ylphenol
Ethane; 2-[(1S,6R)-6-ethyl-3-methylcyclohex-2-en-1-yl]-3-methoxy-5-pentylphenol
(6Ar)-1-methoxy-6,6-dimethyl-9-methylidene-3-(2-methyloctan-2-yl)-7,8,10,10a-tetrahydro-6aH-benzo[c]chromene
(1S,10R)-9,9,13-Trimethyl-5-pentyl-8-oxatetracyclo[8.5.0.01,14.02,7]pentadeca-2,4,6,13-tetraen-3-ol
(6Ar)-6,6,6a,9-tetramethyl-3-pentyl-8,10a-dihydro-7H-benzo[c]chromen-1-ol
6,6-Dimethyl-9-(4-methylpent-4-enyl)-3-pentyl-6a,7,8,10a-tetrahydrobenzo[c]chromen-1-ol; propane
O-[(6Ar,10aR)-6,6,9-trimethyl-3-pentyl-6a,7,8,10a-tetrahydrobenzo[c]chromen-1-yl]hydroxylamine
(6Ar,10aR)-6,6,6a,9-tetramethyl-3-pentyl-10,10a-dihydro-7H-benzo[c]chromen-1-ol
(10Ar)-3-tert-butyl-6,6,9-trimethyl-6a,7,10,10a-tetrahydrobenzo[c]chromen-1-ol
9-Hydroperoxy-9-[(2Z,4E,6Z,8E)-6-methyldeca-2,4,6,8-tetraen-5-yl]xanthene-3,6-diol
2-(5-Methylhex-4-enyl)-7-pentyl-4H-chromen-5-ol
2,6,7-Trimethyl-2-(4-methylpent-3-enyl)-3,4-dihydrochromen-5-ol
(6Ar,10aR)-3-(2-cyclohexylbutan-2-yl)-6,6,9-trimethyl-6a,7,10,10a-tetrahydrobenzo[c]chromen-1-ol
(6Ar,10aR)-3-(1-cycloheptylethyl)-6,6,9-trimethyl-6a,7,10,10a-tetrahydrobenzo[c]chromen-1-ol; ethane
(6Ar)-3-(2,6-dimethyloctan-2-yl)-6,6,9-trimethyl-6a,7,10,10a-tetrahydrobenzo[c]chromen-1-ol
3-(3,7-Dimethyloctan-3-yl)-6,6,9-trimethylbenzo[c]chromen-1-ol
9-[(E)-Hex-1-enyl]xanthene-3,6,9-triol
2-(4,8-Dimethylnona-3,7-dienyl)-2-methyl-7-[(E)-2-phenylethenyl]chromen-5-ol
2-Ethyl-6,6,9-trimethyl-6a,7,8,9,10,10a-hexahydrobenzo[c]chromen-1-ol
(6Ar,10aR)-3-butyl-6,6,6a,9-tetramethyl-8,10a-dihydro-7H-benzo[c]chromen-1-ol
Propylmercury; 3,6,6,9-tetramethyl-6a,7,8,10a-tetrahydrobenzo[c]chromen-1-ol
12-Prop-1-en-2-yl-5-propyl-8-oxatricyclo[7.3.1.02,7]trideca-2,4,6-trien-3-ol
2-Methylpentane; 2-methyl-7-pentyl-3,4-dihydro-2H-chromen-5-ol
2-Methyl-2-(4-methylpent-3-enyl)-7-propyl-3,4-dihydrochromen-5-ol
2-[3-(Hydroxymethyl)cyclohex-2-en-1-yl]-5-pentyl-3-propan-2-yloxyphenol
2-[3-(Hydroxymethyl)cyclohex-2-en-1-yl]-3-propan-2-yloxy-5-propylphenol
9-Methyl-9H-xanthen-3-ol
2-Cyclohexa-1,5-dien-1-yl-5-methyl-4-methylidenechromen-7-ol
Ethane; 6,6,9-trimethyl-3-pentylbenzo[c]chromen-1-ol
Ethane; 6,6,9-trimethyl-3-propylbenzo[c]chromen-1-ol
Ethane; 9-[(3E,5Z)-hepta-1,3,5-trien-3-yl]-9H-xanthene-3,6-diol; methane
[(6Ar)-1-methoxy-6,6-dimethyl-3-(2-methyloctan-2-yl)-6a,7,8,10a-tetrahydrobenzo[c]chromen-9-yl]methanol
Ethane; 3,6,6,8,9-pentamethyl-6a,7,8,9,10,10a-hexahydrobenzo[c]chromen-1-ol
Ethane; 3-methoxy-10a-methyl-9-(2-methylpropyl)-7-propan-2-yl-7,8,8a,9-tetrahydroxanthen-1-ol
(6Ar)-3,6,6,9-tetramethyl-6a,7,8,10a-tetrahydrobenzo[c]chromen-1-ol; methane
6,9-Dimethyl-3-pentyl-6a,7,8,10a-tetrahydro-6H-benzo[c]chromen-1-ol; ethane
6,9-Dimethyl-3-pentyl-6a,7,10,10a-tetrahydro-6H-benzo[c]chromen-1-ol
6,9-Dimethyl-3-pentyl-6a,7,10,10a-tetrahydro-6H-benzo[c]chromen-1-ol; ethane
Ethane; 3-(4-hydroxyphenyl)-5-propyl-3,4-dihydro-2H-chromen-7-ol
[(10As)-1-hydroxy-6,6-dimethyl-3-(2-methyloctan-2-yl)-6a,7,10,10a-tetrahydrobenzo[c]chromen-9-yl]cyanate
(6Ar,10aR)-6,6,9-trimethyl-3-(1,7,7-trimethyl-2-bicyclo[2.2.1]heptanyl)-6a,7,10,10a-tetrahydrobenzo[c]chromen-1-ol; ethane
Ethane; 3,6,6-trimethyl-6a,7,10,10a-tetrahydrobenzo[c]chromen-1-ol
(6Ar,10aR)-3,6,6-trimethyl-6a,7,10,10a-tetrahydrobenzo[c]chromen-1-ol
3-[(2Z,4Z)-Hepta-2,4,6-trienyl]-4-(4-hydroxyphenyl)-2,2-dimethyl-3,4-dihydrochromen-7-ol
3-(1-Hydroxy-6,6,9-trimethyl-6a,7,8,10a-tetrahydrobenzo[c]chromen-3-yl)propanal
9-Hydroperoxy-3-(4-hydroxypentyl)-6,6-dimethyl-6a,7,8,10a-tetrahydrobenzo[c]chromen-1-ol
6,6,9-Trimethyl-3-[(E)-prop-1-enyl]-6a,7,8,10a-tetrahydrobenzo[c]chromen-1-ol
3-[(E)-3-Chloroprop-2-enyl]-6,6,9-trimethyl-6a,7,8,10a-tetrahydrobenzo[c]chromene-1,8-diol
3-(3-Chloropropyl)-6,6,9-trimethyl-6a,7,8,10a-tetrahydrobenzo[c]chromen-1-ol
3,9-Bis(hydroxymethyl)-6,6-dimethyl-6a,7,8,10a-tetrahydrobenzo[c]chromen-1-ol
9-(Hydroxymethyl)-6,6-dimethyl-3-propyl-6a,7,8,10a-tetrahydrobenzo[c]chromen-1-ol
Ethane; 3-(4-hydroxyphenyl)-3,4-dihydro-2H-chromen-7-ol
3-(2-Methyloctan-2-yl)-6a,7,10,10a-tetrahydro-6H-benzo[c]chromen-1-ol
[2-[(6Ar,10aR)-1-hydroxy-6,6,9-trimethyl-6a,7,10,10a-tetrahydrobenzo[c]chromen-3-yl]-2-hydroxyoctyl]formate
3-Methoxy-2-(3-methylbut-3-enyl)-5-[(4-methylcyclohexylidene)methyl]phenol
2-(Hydroxymethyl)-6,6-dimethyl-6a,7,10,10a-tetrahydrobenzo[c]chromen-1-ol
2-(3-Methylcyclohex-2-en-1-yl)-5-pentyl-3-propan-2-yloxyphenol
4-[(6Ar,10aR)-1-[tert-butyl(dimethyl)silyl]oxy-6,6,9-trimethyl-6a,7,10,10a-tetrahydrobenzo[c]chromen-3-yl]decan-4-ol
6,6-Dimethyl-2-(2-methyloctan-2-yl)-6a,7,10,10a-tetrahydrobenzo[c]chromen-1-ol
(6As,10aS)-9-(hydroxymethyl)-6,6-dimethyl-3-(2-methyloctan-2-yl)-6a,7,10,10a-tetrahydrobenzo[c]chromen-1-ol; hydrochloride
3-Ethoxy-2,5-di(propan-2-yl)phenol
5-Butyl-2-methyl-3-octoxyphenol
2,5-Dimethyl-3-octoxyphenol
3-Butoxy-2,5-di(propan-2-yl)phenol
3-Butoxy-2,5-diethylphenol
5-Butyl-3-ethoxy-2-methylphenol
3-Butoxy-5-butyl-2-methylphenol
3-Ethoxy-2-methyl-5-nonylphenol
3-Methoxy-2,5-di(propan-2-yl)phenol
2-Methyl-5-nonyl-3-octoxyphenol
3-Octoxy-2,5-di(propan-2-yl)phenol
2,5-Diethyl-3-octoxyphenol
3-Butoxy-2-methyl-5-nonylphenol
3-Ethoxy-2,5-diethylphenol
4-(7-Hydroxy-2,4-dimethyl-3,4-dihydrochromen-2-yl)-1-methylcyclohexa-3,5-diene-1,3-diol
2-Methyl-2-(4,8,12-trimethyltridecyl)-3,4-dihydrochromene-5,7-diol
6,6,8-Trimethyl-3-pentyl-6a,7,8,10a-tetrahydrobenzo[c]chromen-1-ol
5-Cyclohexyl-3-methoxy-2-methylphenol
5-(8,8-Dimethyl-3,4,9,10-tetrahydro-2H-pyrano[2,3-h]chromen-3-yl)-2-methylphenol
(2S)-2-Methyl-2-(4-methylpent-3-enyl)-7-propylchromen-5-ol
(6As,10aS)-9-(hydroxymethyl)-6,6-dimethyl-3-propyl-6a,7,10,10a-tetrahydrobenzo[c]chromen-1-ol
(6As,10aS)-6,6,9-trimethyl-3-propyl-6a,7,10,10a-tetrahydrobenzo[c]chromen-1-ol
(6As,10aS)-9-(hydroxymethyl)-6,6-dimethyl-3-propyl-6a,7,8,10a-tetrahydrobenzo[c]chromen-1-ol
(6Ar,10aR)-9-(hydroxymethyl)-6,6-dimethyl-3-propyl-6a,7,10,10a-tetrahydrobenzo[c]chromen-1-ol
3-Ethoxy-2-[(6R)-6-ethyl-3-methylcyclohex-3-en-1-yl]-5-propylphenol
(6Ar,10aR)-6,6,6a,9-tetramethyl-3-pentyl-8,10a-dihydro-7H-benzo[c]chromen-1-ol
(1S,9R,11R)-9-Methyl-11-prop-1-en-2-yl-5-propyl-8-oxatricyclo[7.2.2.02,7]trideca-2,4,6-trien-3-ol
2,4,4-Trimethyl-7-pentyl-3a,9b-dihydro-3H-cyclopenta[c]chromen-9-ol
(3Ar)-2,4,4-trimethyl-7-pentyl-3a,9b-dihydro-3H-cyclopenta[c]chromen-9-ol
2-[(3R)-8,8-Dimethyl-3,4,9,10-tetrahydro-2H-pyrano[2,3-h]chromen-3-yl]-5-propylphenol
2-[(3S)-8,8-Dimethyl-3,4,9,10-tetrahydro-2H-pyrano[2,3-h]chromen-3-yl]-5-ethylphenol
2-[(3S)-8,8-Dimethyl-3,4,9,10-tetrahydro-2H-pyrano[2,3-h]chromen-3-yl]-5-propylphenol
2-[(1S,6S)-6-Ethenyl-3-methylcyclohex-2-en-1-yl]-3-methoxy-5-pentylphenol
2-But-3-enyl-2-methyl-7-pentylchromen-5-ol
(2R)-2-Ethenyl-8-ethyl-2,7-dimethyl-3,4-dihydrochromen-5-ol
(2S)-2,7-Dimethyl-8-(3-methylbut-2-enyl)-2-[(1E)-4-methylpenta-1,3-dienyl]-3,4-dihydrochromen-5-ol
5-Methyl-3-(5-methyl-2-propan-2-ylphenoxy)-2-propan-2-ylphenol
(6Ar)-6,6,6a,9-tetramethyl-10,10a-dihydro-7H-benzo[c]chromene-1,3-diol
(6Ar)-6,6,6a,9-tetramethyl-8,10a-dihydro-7H-benzo[c]chromene-1,3-diol
7-Pentyl-3,4-dihydro-2H-chromen-5-ol
7-Butyl-2,2,4-trimethylchromen-5-ol
(6Ar,10aR)-6,6,9-tris[5-(3-methylcyclohex-2-en-1-yl)hexyl]-6a,7,8,9,10,10a-hexahydrobenzo[c]chromen-1-ol
4-(4-Methoxyphenyl)-3,4-dihydro-2H-chromen-7-ol
6,6-Dimethyl-3-propylbenzo[c]chromen-1-ol
7,8,9,10-Tetrahydro-3-pentyl-6,6,10-trimethyl-6H-dibenzo(b,d)pyran-1-ol
[7-[(6Ar,10aR)-1-hydroxy-6,6-dimethyl-6a,7,8,9,10,10a-hexahydrobenzo[c]chromen-3-yl]-7-methyloctyl]nitrate
(6Ar,10aR)-9-(hydroxymethyl)-6,6-dimethyl-3-propyl-6a,7,8,10a-tetrahydrobenzo[c]chromen-1-ol
(6Ar,10aR)-3-[4-[(6aR,10aR)-1-hydroxy-6,9-dimethyl-3-pentyl-6a,7,8,10a-tetrahydrobenzo[c]chromen-6-yl]butyl]-6,6,9-trimethyl-
6a,7,8,10a-tetrahydrobenzo[c]chromen-1-ol
2,3,5-Trimethyl-3,4-dihydro-2H-chromen-7-ol
(6Ar,10aR)-6,6-dimethyl-3-(2-methyloctan-2-yl)-9-sulfanyl-6a,7,10,10a-tetrahydrobenzo[c]chromen-1-ol
(1R)-9,9,13-Trimethyl-5-pentyl-8-oxatetracyclo[8.4.1.01,10.02,7]pentadeca-2,4,6,13-tetraen-3-ol
9-Hexyl-6-methyl-9H-xanthen-3-ol
9-Cyclopentylidene-6-methylxanthen-3-ol
(6Ar)-3,6,6,9-tetramethyl-6a,7,8,10a-tetrahydrobenzo[c]chromen-1-ol
3-(2-Ethoxyethoxy)-2-(3-methyl-6-prop-1-en-2-ylcyclohex-2-en-1-yl)-5-pentylphenol
3-(1,1-Difluoro-2-methylpropan-2-yl)oxy-2-(3-methyl-6-prop-1-en-2-ylcyclohex-2-en-1-yl)-5-pentylphenol
3-(1,3-Difluoro-2-methylpropan-2-yl)oxy-2-(3-methyl-6-prop-1-en-2-ylcyclohex-2-en-1-yl)-5-pentylphenol
3-(1-Fluoro-2-methylpropan-2-yl)oxy-2-(3-methyl-6-prop-1-en-2-ylcyclohex-2-en-1-yl)-5-pentylphenol
3-(1,2-Difluoroethoxy)-2-(3-methyl-6-prop-1-en-2-ylcyclohex-2-en-1-yl)-5-pentylphenol
3-(2-Fluoroethoxy)-2-(3-methyl-6-prop-1-en-2-ylcyclohex-2-en-1-yl)-5-pentylphenol
3-(1-Fluoroethoxy)-2-(3-methyl-6-prop-1-en-2-ylcyclohex-2-en-1-yl)-5-pentylphenol
3-(Fluoromethoxy)-2-(3-methyl-6-prop-1-en-2-ylcyclohex-2-en-1-yl)-5-pentylphenol
2-(3-Methyl-6-prop-1-en-2-ylcyclohex-2-en-1-yl)-5-pentyl-3-propoxyphenol
(6Ar)-3,6,6,9-tetramethyl-6a,7,10,10a-tetrahydrobenzo[c]chromen-1-ol
(6As,10aS)-6,6,9-trimethyl-3-pentyl-8,10a-dihydro-7H-benzo[c]chromene-1,6a-diol
2-[(2R)-5-Methoxy-2,7-dimethyl-3,4-dihydrochromen-2-yl]ethanol
(2S)-2-[2-[(1S)-2,2-Dimethyl-6-methylidenecyclohexyl]ethyl]-5-methoxy-2,7-dimethyl-3,4-dihydrochromene
(6Ar,9R,10aR)-6,6,9-trimethyl-3-[(E)-2-phenylethenyl]-6a,7,8,9,10,10a-hexahydrobenzo[c]chromen-1-ol
2,2-Dimethyl-3-[(1E)-3-methylbuta-1,3-dienyl]-7-pentyl-3,4-dihydrochromen-5-ol
[(9S,10R)-1-Methoxy-9-methyl-3-pentyl-8,9,10,10a-tetrahydro-6H-benzo[c]chromen-10-yl]methanol
(6Ar,10aR)-9-fluoro-6,6-dimethyl-3-pentyl-6a,7,10,10a-tetrahydrobenzo[c]chromen-1-ol
[(6Ar,10aR)-6,6,9-trimethyl-3-pentyl-6a,7,10,10a-tetrahydrobenzo[c]chromen-1-yl]oxy-tri(propan-2-yl)silane
3-(2-Bromo-3-methylbut-3-enyl)-2,2-dimethyl-7-pentyl-3,4-dihydrochromen-5-ol
(6S,6Ar,10aR)-6,9-dimethyl-3-pentyl-6a,7,10,10a-tetrahydro-6H-benzo[c]chromen-1-ol
O-[(6Ar,10aR)-1-hydroxy-6,6,9-trimethyl-3-pentyl-6a,7,10,10a-tetrahydrobenzo[c]chromen-10-yl]methylsulfanylmethanethioate
3-[(E)-2-(5-Methoxy-2,2-dimethylchromen-7-yl)ethenyl]phenol
2,2-Dimethyl-6-(3-methylbut-2-enyl)-3,4-dihydrochromen-5-ol
(6Ar,10aR)-6,6,9-trimethyl-3-pentyl-6a,7,8,10a-tetrahydrobenzo[c]chromen-1-ol; 2-[(1R,6R)-3-methyl-6-prop-1-en-2-ylcyclohex-2-
en-1-yl]-5-pentylbenzene-1,3-diol; (1E,4E,8E)-2,6,6,9-tetramethylcycloundeca-1,4,8-triene; (1R,4E,9S)-4,11,11-trimethyl-8-
methylidenebicyclo[7.2.0]undec-4-ene
2-Hex-4-ynyl-2-methyl-7-pentylchromen-5-ol
2-(3-Methyl-6-prop-1-en-2-ylcyclohex-2-en-1-yl)-5-pentyl-3-phenoxyphenol
2-(3-Methyl-6-prop-1-en-2-ylcyclohex-2-en-1-yl)-5-pentyl-3-(1,1,1-trifluoro-2-methylpropan-2-yl)oxyphenol
2-(3-Methyl-6-prop-1-en-2-ylcyclohex-2-en-1-yl)-5-pentyl-3-(trifluoromethoxy)phenol
3-Ethoxy-2-(3-methyl-6-prop-1-en-2-ylcyclohex-2-en-1-yl)-5-pentylphenol
2-(3-Methyl-6-prop-1-en-2-ylcyclohex-2-en-1-yl)-5-pentyl-3-[2-(2-prop-2-enoxyethoxy)ethoxy]phenol
(1R,9R,11R)-9-Methyl-11-prop-1-en-2-yl-5-propyl-8-oxatricyclo[7.2.2.02,7]trideca-2,4,6-trien-3-ol
6-Methoxy-9-propan-2-yl-9H-xanthen-3-ol
5-(8,8-Dimethyl-3,4,9,10-tetrahydro-2H-pyrano[2,3-h]chromen-3-yl)-2-propylphenol
2-[(3R)-8,8-Dimethyl-3,4,9,10-tetrahydro-2H-pyrano[2,3-h]chromen-3-yl]-5-ethylphenol
6,6,6a,9-Tetramethyl-3-pentyl-8,10a-dihydro-7H-benzo[c]chromen-1-ol
(1S,11R)-3,13-Dimethyl-7-pentyl-4-oxatetracyclo[9.4.0.01,3.05,10]pentadeca-5,7,9,12-tetraen-9-ol
(2S)-2-Methyl-2-[(E)-4-methylpent-2-enyl]-7-propylchromen-5-ol
3-(4-Methylphenyl)-3,4-dihydro-2H-chromen-7-ol
(7R)-6,6,9-Trimethyl-3-pentyl-6a,7,8,10a-tetrahydrobenzo[c]chromene-1,7-diol
4-Methyl-2-methylidene-7-propan-2-ylchromen-5-ol
4-Methyl-2-methylidene-5-propan-2-ylchromen-7-ol
(3R,4S)-4-[(3Z)-5,6-Dimethylhepta-1,3,5-trien-2-yl]-3-(4-hydroxyphenyl)-3,4-dihydro-2H-chromen-7-ol
[3-Hydroxy-2-(3-methyl-6-prop-1-en-2-ylcyclohex-2-en-1-yl)-5-pentylphenyl]acetate
(6,6,9-Trimethyl-3-propyl-6a,7,8,10a-tetrahydrobenzo[c]chromen-1-yl)acetate
2,2,3-Trimethyl-4-(2-methylprop-1-enyl)-8-pentyl-5H-1-benzoxepin-6-ol
CID131728058
(6Ar,10aR)-9-methyl-6-methylidene-3-pentyl-6a,7,8,10a-tetrahydrobenzo[c]chromen-1-ol
4-Ethyl-3,4-dihydro-2H-chromen-5-ol
9-Methyl-6-methylidene-3-propylbenzo[c]chromen-1-ol
(6Ar)-6,9-dimethyl-3-pentyl-6a,7,8,10a-tetrahydro-6H-benzo[c]chromen-1-ol
(6Ar)-6,6,9-trimethyl-3-propyl-6a,7,10,10a-tetrahydrobenzo[c]chromen-1-ol
2,3,4-Trimethyl-3,4-dihydro-2H-chromene-5,7-diol
(6Ar,10aR)-6,6,9-trimethyl-1-methylperoxy-3-pentyl-6a,7,8,10a-tetrahydrobenzo[c]chromene
3-Methoxy-2-[(6R)-3-methyl-6-prop-1-en-2-ylcyclohex-2-en-1-yl]-5-pentylphenol
(6Ar,10aR)-3-[4-[(6aR,10aR)-1-hydroxy-3,6,9-trimethyl-6a,7,8,10a-tetrahydrobenzo[c]chromen-6-yl]butyl]-6,6,9-trimethyl-
6a,7,8,10a-tetrahydrobenzo[c]chromen-1-ol
2,6-Dimethyl-2-(4-methylpent-3-enyl)-7-pentylchromen-5-ol
4-[(6Ar,10aR)-1-hydroxy-6,6,9-trimethyl-6a,7,10,10a-tetrahydrobenzo[c]chromen-3-yl]pentylnitrate
(6Ar,10aR)-1-ethylperoxy-6,6,9-trimethyl-3-pentyl-6a,7,8,10a-tetrahydrobenzo[c]chromene
(6Ar,10aR)-6,6,9-trimethyl-1-methylsulfanyloxyperoxy-3-pentyl-6a,7,8,10a-tetrahydrobenzo[c]chromene
(6Ar,10aR)-1-ethoxy-6,6,9-trimethyl-3-pentyl-6a,7,8,10a-tetrahydrobenzo[c]chromene
5-Tert-butyl-2-cyclohexyl-3-cyclohexyloxyphenol
2,4,5-Trimethyl-8-pent-3-en-2-yl-3,4-dihydro-2H-chromen-7-ol
5-Methyl-2-pent-3-en-2-yl-3-pent-3-en-2-yloxyphenol
3-(4-Hydroxybutyl)-6,6,9-trimethyl-6a,7,8,10a-tetrahydrobenzo[c]chromen-1-ol
(10As)-6,6,9-trimethyl-3-pentyl-6a,7,10,10a-tetrahydrobenzo[c]chromen-1-ol
9-(Hydroxymethyl)-6,6,6a-trimethyl-3-(2-methyloctan-2-yl)-10,10a-dihydro-7H-benzo[c]chromen-1-ol
3-[2-[(1R)-4-(Hydroxymethyl)cyclohex-3-en-1-yl]propan-2-yloxy]-2-methyl-5-(2-methyloctan-2-yl)phenol
(2S)-2-Ethyl-2-methyl-5-prop-1-en-2-yl-8-propyl-4,5-dihydro-3H-1-benzoxepin-6-ol
(9R)-9-Methyl-1-prop-1-en-2-yl-5-propyl-8-oxatricyclo[7.3.1.02,7]trideca-2,4,6-trien-3-ol
(6Ar,10aR)-6-ethyl-6,9-dimethyl-3-pentyl-6a,7,8,10a-tetrahydrobenzo[c]chromen-1-ol
(6Ar,10aR)-3-(8-bromo-2-methyloctan-2-yl)-6,6,9-trimethyl-6a,7,8,10a-tetrahydrobenzo[c]chromen-1-ol
3-Propan-2-yl-3,4-dihydro-2H-chromene-5,7-diol
(10Ar)-3-(1,1-difluoropentyl)-6,6,9-trimethyl-6a,7,8,10a-tetrahydrobenzo[c]chromen-1-ol
(9R)-9-Ethyl-6,6-dimethyl-3-pentyl-6a,7,8,9,10,10a-hexahydrobenzo[c]chromen-1-ol
6,6,9-Trimethyl-3-pentyl-6a,10a-dihydrobenzo[c]chromen-1-ol
(6As,10aR)-6,6,9-trimethyl-3-propyl-6a,7,8,10a-tetrahydrobenzo[c]chromen-1-ol
(6As,10aS)-6,6,9-trimethyl-3-propyl-6a,7,8,10a-tetrahydrobenzo[c]chromen-1-ol
2-(3-Methylcyclohex-2-en-1-yl)-3-propan-2-yloxy-5-propylphenol
(10Ar)-6,6,9-trimethyl-3-propyl-6a,7,8,10a-tetrahydrobenzo[c]chromen-1-ol
2,4,6,6,9-Pentamethyl-3-pentyl-6a,7,8,10a-tetrahydrobenzo[c]chromen-1-ol
2-[(3R)-8,8-Dimethyl-3,4-dihydro-2H-pyrano[2,3-f]chromen-3-yl]-5-methylphenol
3-Butoxy-2,5-dicyclopropylphenol
3-(4-Tert-butylphenyl)-5-[(2-methylpropan-2-yl)oxy]phenol
2-(3-Hydroxy-2-methyl-5-propan-2-ylphenoxy)-1-methyl-4-propan-2-ylcyclohex-4-ene-1,3-diol
9-(Hydroxymethyl)-6,6-dimethyl-6a,7,8,9,10,10a-hexahydrobenzo[c]chromen-1-ol
7-(2-Cyclohexa-1,3-dien-1-ylethenyl)-5-methoxy-2,2-dimethyl-3,4-dihydrochromene
9-Hexyl-2,4,5,7-tetramethyl-9H-xanthene-3,6-diol
5-(1-Hydroxy-2-methylpropan-2-yl)-2-(3-methylcyclohex-3-en-1-yl)-3-propan-2-yloxyphenol
2,7-Diethyl-4-methyl-9-propan-2-yl-9H-xanthene-3,6-diol
2-(4-Methoxycyclohexyl)-3,5-dimethyl-8-(3-methylbutyl)-4-methylidene-2,3-dihydrochromen-7-ol
2,2-Dimethyl-4-(2-methylpent-1-enyl)-7-pentyl-3,4-dihydrochromen-5-ol
2,4,7-Trimethyl-9-propan-2-yl-9H-xanthene-3,6-diol
2,2,6,7-Tetramethylchromen-5-ol
3-(1,1-Difluoropentyl)-6,6,9-trimethyl-6a,7,10,10a-tetrahydrobenzo[c]chromen-1-ol
2-(6-Ethenyl-3-methylcyclohex-2-en-1-yl)-5-heptyl-3-methoxyphenol
5-Hept-1-enyl-3-methoxy-2-(3-methylcyclohex-2-en-1-yl)phenol
(2R)-2-Methyl-2-(4-methylpentyl)-7-pentyl-3,4-dihydrochromen-5-ol
2,4,5,7-Tetramethyl-9-propan-2-yl-9H-xanthene-3,6-diol
2-(3-Hydroxy-4-methylphenyl)-3,4-dihydro-2H-chromen-7-ol
6,6,9-Trimethyl-3-propyl-7,8,10a,11-tetrahydro-6aH-benzo[c][1]benzoxepin-1-ol
5-Ethyl-2-[3-methyl-6-[1-(1-methyl-4-prop-1-en-2-ylcyclohex-2-en-1-yl)oxyethenyl]cyclohex-2-en-1-yl]benzene-1,3-diol
(6Ar,10aR)-2-(hydroxymethyl)-6,6,9-trimethyl-3-pentyl-6a,7,8,9,10,10a-hexahydrobenzo[c]chromen-1-ol
2-(8,8-Diethyl-3,4,9,10-tetrahydro-2H-pyrano[2,3-h]chromen-3-yl)-5-methylphenol
2-(8,8-Dimethyl-3,4,9,10-tetrahydro-2H-pyrano[2,3-h]chromen-3-yl)-5-propan-2-ylphenol
2-(8,8-Dimethyl-3,4,9,10-tetrahydro-2H-pyrano[2,3-h]chromen-3-yl)-5-pentylphenol
5-Butyl-2-(8,8-dimethyl-3,4,9,10-tetrahydro-2H-pyrano[2,3-h]chromen-3-yl)phenol
(1R,9R)-9-Methyl-12-prop-1-en-2-yl-5-propyl-8-oxatricyclo[7.3.1.02,7]trideca-2,4,6-trien-3-ol
(1S,9S,12R)-9-Methyl-12-prop-1-en-2-yl-5-propyl-8-oxatricyclo[7.3.1.02,7]trideca-2,4,6-trien-3-ol
(3R,4R)-3-(2-Hydroxyethyl)-4-(hydroxymethyl)-2,2-dimethyl-7-(2-methyloctan-2-yl)-3,4-dihydrochromen-5-ol
(3S)-3-(4-Methylphenyl)-3,4-dihydro-2H-chromen-7-ol
(6Ar,10aR)-1-methoxy-6,6-dimethyl-9-methylidene-3-octan-2-yl-7,8,10,10a-tetrahydro-6aH-benzo[c]chromene
(6Ar,10aR)-1-methoxy-6,6,9-trimethyl-3-octan-2-yl-6a,7,10,10a-tetrahydrobenzo[c]chromene
2,6,7,8-Tetramethyl-2-(4-methylpent-3-enyl)-3,4-dihydrochromen-5-ol
[(6Ar,9R,10aR)-3-heptyl-1-hydroxy-6,6-dimethyl-6a,7,8,9,10,10a-hexahydrobenzo[c]chromen-9-yl]methyl nitrate
(6Ar,10aR)-6,6-dimethyl-3-(2-methylnonan-2-yl)-6a,7,8,9,10,10a-hexahydrobenzo[c]chromene-1,9-diol
(6As,10aS)-9-(hydroxymethyl)-6,6-dimethyl-3-(2-methylnonan-2-yl)-6a,7,10,10a-tetrahydrobenzo[c]chromen-1-ol
(6Ar,10aR)-9-(hydroxymethyl)-6,6-dimethyl-3-(2-methylnonan-2-yl)-6a,7,10,10a-tetrahydrobenzo[c]chromen-1-ol
(6Ar,9R,10aR)-9-(hydroxymethyl)-6,6-dimethyl-3-(2-methylnonan-2-yl)-6a,7,8,9,10,10a-hexahydrobenzo[c]chromen-1-ol
9-Methoxy-6,6-dimethyl-3-(2-methylnonan-2-yl)benzo[c]chromen-1-ol
3-[(6Ar,10aR)-1-hydroxy-6,6,9-trimethyl-6a,7,10,10a-tetrahydrobenzo[c]chromen-3-yl]propyl nitrate
[4-[(6Ar,10aR)-1-hydroxy-9-(hydroxymethyl)-6,6-dimethyl-6a,7,8,10a-tetrahydrobenzo[c]chromen-3-yl]-4-methylpentyl] nitrate
4-[(6Ar,10aR)-1-hydroxy-6,6,9-trimethyl-6a,7,8,10a-tetrahydrobenzo[c]chromen-3-yl]butyl nitrate
[6-[(6Ar,10aR)-1-hydroxy-6,6,9-trimethyl-6a,7,8,10a-tetrahydrobenzo[c]chromen-3-yl]-6-methylheptyl] nitrate
[6-[(6Ar,10aR)-1-hydroxy-9-(hydroxymethyl)-6,6-dimethyl-6a,7,10,10a-tetrahydrobenzo[c]chromen-3-yl]-6-methylheptyl] nitrate
5-[(6Ar,10aR)-1-hydroxy-6,6,9-trimethyl-6a,7,10,10a-tetrahydrobenzo[c]chromen-3-yl]pentyl nitrate
[3-[(6Ar,10aR)-1-hydroxy-6,6,9-trimethyl-6a,7,10,10a-tetrahydrobenzo[c]chromen-3-yl]-3-methylbutyl] nitrate
[7-[(6Ar,10aR)-1-hydroxy-6,6,9-trimethyl-6a,7,8,10a-tetrahydrobenzo[c]chromen-3-yl]-7-methyloctyl] nitrate
5-[(6As,10aS)-1-hydroxy-9-methyl-6a,7,10,10a-tetrahydro-6H-benzo[c]chromen-3-yl]pentyl nitrate
[7-[(6Ar,10aR)-1-hydroxy-9-(hydroxymethyl)-6,6-dimethyl-6a,7,8,10a-tetrahydrobenzo[c]chromen-3-yl]-7-methyloctyl] nitrate
[5-[(6Ar,10aR)-1-hydroxy-6,6,9-trimethyl-6a,7,8,10a-tetrahydrobenzo[c]chromen-3-yl]-5-methylhexyl] nitrate
6-[(6Ar,10aR)-1-hydroxy-6,6,9-trimethyl-6a,7,10,10a-tetrahydrobenzo[c]chromen-3-yl]hexyl nitrate
[3-[(6Ar,10aR)-1-hydroxy-9-(hydroxymethyl)-6,6-dimethyl-6a,7,8,10a-tetrahydrobenzo[c]chromen-3-yl]-3-methylbutyl] nitrate
9-[(6Ar,10aR)-1-hydroxy-6,6,9-trimethyl-6a,7,8,10a-tetrahydrobenzo[c]chromen-3-yl]nonyl nitrate
[8-[(6Ar,10aR)-1-hydroxy-6,6,9-trimethyl-6a,7,10,10a-tetrahydrobenzo[c]chromen-3-yl]-8-methylnonyl] nitrate
5-[(6As,10aR)-1-hydroxy-9-methyl-6a,7,8,10a-tetrahydro-6H-benzo[c]chromen-3-yl]pentyl nitrate
(6Ar,9E,10aR)-3-(8-bromo-2-methyloctan-2-yl)-9-(methoxymethylidene)-6,6-dimethyl-7,8,10,10a-tetrahydro-6aH-
benzo[c]chromen-1-ol
2-[(6Ar,10aR)-1-hydroxy-6,6,9-trimethyl-6a,7,10,10a-tetrahydrobenzo[c]chromen-3-yl]ethyl nitrate
6-[(6Ar,10aR)-1-hydroxy-6,6,9-trimethyl-6a,7,8,10a-tetrahydrobenzo[c]chromen-3-yl]hexyl nitrate
[(6Ar,10aR)-1-hydroxy-6,6-dimethyl-3-(2-methylnonan-2-yl)-6a,7,10,10a-tetrahydrobenzo[c]chromen-9-yl]methyl nitrate
[5-[(6As,10aS)-1-hydroxy-9-(hydroxymethyl)-6,6-dimethyl-6a,7,8,10a-tetrahydrobenzo[c]chromen-3-yl]-5-methylhexyl] nitrate
[4-[(6Ar,10aR)-1-hydroxy-9-(hydroxymethyl)-6,6-dimethyl-6a,7,10,10a-tetrahydrobenzo[c]chromen-3-yl]-4-methylpentyl] nitrate
[3-[(6Ar,10aR)-1-hydroxy-9-(hydroxymethyl)-6,6-dimethyl-6a,7,10,10a-tetrahydrobenzo[c]chromen-3-yl]-3-methylbutyl] nitrate
[2-[(6Ar,10aR)-1-hydroxy-6,6,9-trimethyl-6a,7,8,10a-tetrahydrobenzo[c]chromen-3-yl]-2-methylpropyl] nitrate
9-[(6Ar,10aR)-1-hydroxy-6,6,9-trimethyl-6a,7,10,10a-tetrahydrobenzo[c]chromen-3-yl]nonyl nitrate
8-[(6Ar,10aR)-1-hydroxy-6,6,9-trimethyl-6a,7,10,10a-tetrahydrobenzo[c]chromen-3-yl]nonyl nitrate
[4-[(6Ar,10aR)-1-hydroxy-6,6,9-trimethyl-6a,7,8,10a-tetrahydrobenzo[c]chromen-3-yl]-4-methylpentyl] nitrate
[6-[(6As,10aS)-1-hydroxy-9-(hydroxymethyl)-6,6-dimethyl-6a,7,8,10a-tetrahydrobenzo[c]chromen-3-yl]-6-methylheptyl] nitrate
[4-[(6Ar,10aR)-1-hydroxy-6,6,9-trimethyl-6a,7,10,10a-tetrahydrobenzo[c]chromen-3-yl]-4-methylpentyl] nitrate
[5-[(6Ar,10aR)-1-hydroxy-9-(hydroxymethyl)-6,6-dimethyl-6a,7,10,10a-tetrahydrobenzo[c]chromen-3-yl]-5-methylhexyl] nitrate
3-[(6Ar,10aR)-1-hydroxy-6,6,9-trimethyl-6a,7,8,10a-tetrahydrobenzo[c]chromen-3-yl]propyl nitrate
[3-[(6Ar,10aR)-1-hydroxy-6,6,9-trimethyl-6a,7,8,10a-tetrahydrobenzo[c]chromen-3-yl]-3-methylbutyl] nitrate
[6-[(6Ar,10aR)-1-hydroxy-6,6,9-trimethyl-6a,7,10,10a-tetrahydrobenzo[c]chromen-3-yl]-6-methylheptyl] nitrate
8-[(6Ar,10aR)-1-hydroxy-6,6,9-trimethyl-6a,7,10,10a-tetrahydrobenzo[c]chromen-3-yl]octyl nitrate
4-[(6Ar,10aR)-1-hydroxy-6,6,9-trimethyl-6a,7,10,10a-tetrahydrobenzo[c]chromen-3-yl]butyl nitrate
7-[(6Ar,10aR)-1-hydroxy-6,6,9-trimethyl-6a,7,8,10a-tetrahydrobenzo[c]chromen-3-yl]heptyl nitrate
8-[(6Ar,10aR)-1-hydroxy-6,6,9-trimethyl-6a,7,8,10a-tetrahydrobenzo[c]chromen-3-yl]octyl nitrate
[5-[(6Ar)-1-hydroxy-6,6,9-trimethyl-6a,7,10,10a-tetrahydrobenzo[c]chromen-3-yl]-5-methylhexyl] nitrate
[7-[(6As,10aS)-1-hydroxy-9-(hydroxymethyl)-6,6-dimethyl-6a,7,10,10a-tetrahydrobenzo[c]chromen-3-yl]-7-methyloctyl] nitrate
2-[(6Ar,10aR)-1-hydroxy-6,6,9-trimethyl-6a,7,8,10a-tetrahydrobenzo[c]chromen-3-yl]ethyl nitrate
(6Ar,10aR)-3-(6-iodo-2-methylhexan-2-yl)-6,6,9-trimethyl-6a,7,10,10a-tetrahydrobenzo[c]chromen-1-ol
7-[(6Ar,10aR)-1-hydroxy-6,6,9-trimethyl-6a,7,10,10a-tetrahydrobenzo[c]chromen-3-yl]heptyl nitrate
[7-[(6Ar,10aR)-1-hydroxy-6,6,9-trimethyl-6a,7,10,10a-tetrahydrobenzo[c]chromen-3-yl]-7-methyloctyl] nitrate
(6Ar,9R,10aR)-9-(bromomethyl)-3-heptyl-6,6-dimethyl-6a,7,8,9,10,10a-hexahydrobenzo[c]chromen-1-ol
6-[(6Ar,10aR)-1-hydroxy-6,6,9-trimethyl-6a,7,8,10a-tetrahydrobenzo[c]chromen-3-yl]heptyl nitrate
5-(1-Hydroxy-6,6,9-trimethyl-6a,7,8,10a-tetrahydrobenzo[c]chromen-3-yl)pentyl nitrate
[8-[(6Ar,10aR)-1-hydroxy-6,6,9-trimethyl-6a,7,8,10a-tetrahydrobenzo[c]chromen-3-yl]-8-methylnonyl] nitrate
[2-[(6Ar,10aR)-1-hydroxy-6,6,9-trimethyl-6a,7,10,10a-tetrahydrobenzo[c]chromen-3-yl]-2-methylpropyl] nitrate
2-Methyl-7-propyl-2-[(E)-4-sulfanylpent-3-enyl]chromen-5-ol
3-Butyl-6,6,9-trimethyl-8,10a-dihydro-7H-benzo[c]chromene-1,6a,7-triol
(1R,12R)-9-Methyl-12-prop-1-en-2-yl-5-propyl-8-oxatricyclo[7.3.1.02,7]trideca-2,4,6-trien-3-ol
(10R)-6,6,9-Trimethyl-3-pentyl-8,10-dihydro-7H-benzo[c]chromene-1,8,9,10-tetrol
(12R)-9-Methyl-5-pentyl-12-propan-2-yl-8-oxatricyclo[7.3.1.02,7]trideca-2,4,6-trien-3-ol
(6S,6Ar,10aR)-6,9-dimethyl-3-pentyl-6a,7,8,10a-tetrahydro-6H-benzo[c]chromen-1-ol
2,2-Dimethyl-7-propan-2-yl-3,4-dihydrochromen-5-ol
2,2-Dimethyl-7-propyl-3,4-dihydrochromen-5-ol
(6Ar,10aR)-1-hydroxy-6,6-dimethyl-3-(513C)pentyl-7,8,10,10a-tetrahydro-6aH-benzo[c]chromen-9-one
(6Ar,10aR)-6,6,9-trimethyl-3-(513C)pentyl-6a,7,8,9,10,10a-hexahydrobenzo[c]chromen-1-ol
2,10,10-Trimethyl-6-pentyl-3,9-dioxatetracyclo[6.4.2.04,14.011,13]tetradeca-1,4(14),5,7-tetraene
(1S,9As)-1-ethyl-3a-methyl-6-pentyl-2,3,9,9a-tetrahydro-1H-cyclopenta[b]chromen-8-ol
(10R)-6,6,9-Trimethyl-3-pentyl-7,8,9,10-tetrahydrobenzo[c]chromene-1,10-diol
2-(8,8-Dimethyl-3,4,9,10-tetrahydro-2H-pyrano[2,3-h]chromen-3-yl)-5-ethylphenol
2-(8,8-Dimethyl-3,4,9,10-tetrahydro-2H-pyrano[2,3-h]chromen-3-yl)-5-(2-methoxyethyl)phenol
2-(8,8-Dimethyl-3,4,9,10-tetrahydro-2H-pyrano[2,3-h]chromen-3-yl)-5-methylphenol
3-Methoxy-6-methyl-9-propan-2-yl-9H-xanthene
9-Hexyl-3-methoxy-6-methyl-9H-xanthene
(4S)-2,2-Dimethyl-4-[(Z)-2-methylpent-1-enyl]-7-pentyl-3,4-dihydrochromen-5-ol
(5Z,8S)-2,2,6,8-Tetramethyl-11-pentyl-3,4,7,8-tetrahydro-1-benzoxecin-9-ol
2-[(Z,2S)-4-Methylhept-4-en-2-yl]-3-[(2-methylpropan-2-yl)oxy]-5-pentylphenol

In certain embodiments, the molecule is selected from any of the molecules in the table below:

Amphetamine
Amphetamine sulfate
Amphetamine hydrochloride
Levamfetamine
D-Amphetamine-d3 hemisulfate
Amphetamine chlorphenoxyacetate
(+/−)-Amphetamine-d8
Amphetamine-d6
Amphetamine, N-propoxycarbonyl-
Amphetamine D-3 monosulfate, (+)-
Amphetamine phosphate
N-(2-Dansylaminoethyl)amphetamine
Amphetamine PFP
Amphetamine-propionyl
2,5-Dimethoxy-4-(methylthionyl)amphetamine
Amphetamine-d11
Amphetamine aspartate monohydrate
2,5-Dimethoxy-4-(methylsulfonyl)amphetamine
4-Propoxy-2,5-dimethoxy amphetamine
Amphetamine oxime acetate
Amphetamine methanesulfonate
Amphetamine-3-methyl acetate
4-(2-Hydroxypropoxy)amphetamine
N-Ethyl-N-(4-carbethoxyhexafluorobutyryl)-amphetamine
Amphetamine analog
N-Carboxyethyl-amphetamine
4-(2-Pyridylmethoxy)-amphetamine
(S)-Amphetamine Hydrochloride
N-Acetyl-4-(2-hydroxypropoxy)amphetamine
Amphetamine, N-butoxycarbonyl-
Amphetamine tartrate
Amphetamine, N-4-carbethoxyhexafluorobutyryl deriv.
Amphetamine-d7
N-Ethyl-p-chloro-amphetamine
N-Methacryloyl-DL-amphetamine, copolymer with vinyl acetate
4-Ethoxy-N-(1-methylacetonyl)amphetamine
(R)-3,4-Dimethoxy-amphetamine hydrochloride
Amphetamine adipate
N-Methyl-N-n-butyl-amphetamine
N-Trifluoroacetyl amphetamine
2,5-Dimethoxy-4-(2-fluoroethyl)amphetamine
Dextroamphetamine
Dextroamphetamine sulfate
Adzenys XR-ODT
Tenamfetamine hydrochloride
Amphedrine
Etilamfetamine
4-(2-Aminopropyl)phenol
Dextroamphetamine hydrochloride
CID 129318245
3,4,5-Trimethoxyamphetamine hydrochloride
Silylamine, 1,1,1-trimethyl-N-(alpha-methylphenethyl)-
CID 129318241
Unii-Q5L4P8K8QZ
1,1,1,2,3-Pentadeuterio-3-phenylpropan-2-amine
N-[Tert-butyl(dimethyl)silyl]-1-phenylpropan-2-amine
3,4-Dimethoxyamphetamine hydrochloride
CID 129318244
1-Methyl-2-phenylethylamine N-ethylpiperazine
Unii-1N32846021
[(1R,2R,4S,5S)-9-Methyl-3-oxa-9-azatricyclo[3.3.1.02,4]nonan-7-yl] (2S)-3-hydroxy-2-phenylpropanoate;
1-phenylpropan-2-amine
Tenamfetamine
CID 46780382
Fenethylline
3-(2-Aminopropyl)phenol
1-(2,3-Dimethoxy-4-methylphenyl)propan-2-amine
2-[5-(2-Aminopropyl)-2-hydroxyphenyl]ethane-1,1-diol
2,4,5-Trimethoxyamphetamine hydrochloride
N-(1-Phenylpropan-2-yl)-1-(2,2,2-trifluoroacetyl)pyrrolidine-2-carboxamide
9H-Fluoren-9-ylmethyl N-(1-phenylpropan-2-yl)carbamate
N-Ethoxy-N,2-dimethoxy-1-phenylpropan-2-amine
2,5-Dimethoxyamphetamine
1-(4-Fluorophenyl)propan-2-amine hydrochloride
2-Fluoroamphetamine
6-(2-Aminopropyl)-1,3-benzodioxol-5-ol
N,N-Dipropylamphetamine
Methyl N-(1-phenylpropan-2-yl)carbamate
Lisdexamfetamine dimesylate
4-Fluoromethamphetamine
CID 19261721
1-Phenylpropan-2-amine; hydrate
(2S)-5-Oxopyrrolidine-2-carboxylic acid; 1-phenylpropan-2-amine
1-Phenylpropan-2-amine; dihydrochloride
Lisdexamphetamine
N-Methoxy-N-methyl-1-phenylpropan-2-amine
CID 129318243
N,2-Dimethoxy-N-methyl-1-phenylbutan-2-amine
1,1,1-Trimethoxy-3-phenylpropan-2-amine;hydrochloride
N,N,1,2-Tetradeuterio-3-phenylpropan-2-amine
N-Bromo-N-methoxy-1-phenylpropan-2-amine
(2S)-2,6-Diaminohexanoic acid; (2S)-1-phenylpropan-2-amine
Gold(3+); (2S)-1-phenylpropan-2-amine; dichloride
2-Methoxy-4-methylamphetamine hydrochloride
2,4-Dimethoxy-3-methylamphetamine hydrochloride
N-Propylamphetamine
2,5-Dimethoxy-4-nitroamphetamine
4-Bromo-3,5-dimethoxyamphetamine
Benzeneethanamine, 3-bromo-2,5-dimethoxy-alpha-methyl-
1-(2,3,4,5,6-Pentadeuteriophenyl)propan-2-amine
N-(1-Phenylpropan-2-yl)pentan-1-amine
1-(5-Fluoro-2-methoxyphenyl)propan-2-amine
1-(1-Methylcyclohexa-2,4-dien-1-yl)propan-2-amine
(2-Amino-3-phenylpropyl) 2-oxochromene-3-carboxylate
1,1,1,2,3-Pentadeuterio-3-(2,3,4,5,6-pentadeuteriophenyl)propan-2-amine
1-Phenyl-N-(trifluoromethyl)propan-2-amine
2-[4-(2-Aminopropyl)-2-hydroxyphenyl]acetic acid
[(2S)-1-Phenylpropan-2-yl]azanium; bromide
4-Fluoroamphetamine
Fenproporex
2,5-Dimethoxyamphetamine hydrochloride
2,5-Dimethoxy-4-methylamphetamine hydrochloride
4-(2-Aminopropyl)-2,6-xylenol hydrochloride
Lisdexamfetamine
(+)-2,5-Dimethoxyamphetamine
1-(2,5-Dimethoxy-4-tert-butylphenyl)-2-propanamine
(2S)-N-[(2S)-3-(1H-Imidazol-5-yl)-1-oxo-1-(1-phenylpropan-2-ylamino)propan-2-yl]-5-oxopyrrolidine-2-carboxamide
N-Methyl-N-(1-phenylpropan-2-yl) aniline
1-[1-(3,4-Dimethoxyphenyl)prop-2-ylamino]1-phenylethane
N-(1-Phenylpropan-2-yl)thiohydroxylamine
2,6-Diamino-4,4,5,5-tetradeuterio-N-[(2S)-1-phenylpropan-2-yl]hexanamide; dihydrochloride
Benzamide; (2S)-1-phenylpropan-2-amine
[[(2R,3S,4R,5R)-5-(6-Aminopurin-9-yl)-3,4-dihydroxyoxolan-2-yl]methoxy-hydroxyphosphoryl] phosphono
hydrogen phosphate; 1-phenylpropan-2-amine
1-(1,3-Benzodioxol-5-yl)-N-trimethylsilyloxypropan-2-amine
N,N,1,1,1,2,3,3-Octadeuterio-3-phenylpropan-2-amine
N,2-Dimethoxy-N-methyl-1-phenylpropan-2-amine
1-Cyclohexa-1,3-dien-1-ylpropan-2-amine
5-(2-Aminopropyl)-2-(2-hydroxyethyl)phenol
3,4-Methylenedioxymethamphetamine
Methamphetamine
1-(2,3,4-Trimethoxyphenyl)propan-2-amine
1-(3-Methoxy-4-methylphenyl)-2-aminopropane
3,4-Methylenedioxy-N-ethylamphetamine
3-Fluoroamphetamine
4-(2-(Ethylamino)propyl)-1,2-benzenediol hydrobromide
alpha,2-Dimethyl-1,3-benzodioxole-5-ethanamine
2,5-Dimethoxy-4-methylthioamphetamine
DL-2-Methoxyamino-1-(4-methyl-phenyl)-propan
N-Ethyl-N-methylamphetamine
(S)-(−)-N-Benzyl-1-phenylethylamine
(R)-alpha-Methyl-1,3-benzodioxole-5-ethanamine
(S)-alpha-Methyl-1,3-benzodioxole-5-ethanamine
4-(2-Aminopropyl)-3-chloroaniline
4-[(2R)-2-Aminopropyl]phenol
Ethyl N-(1-phenylpropan-2-yl)carbamate
N,N,1,1,2-Pentadeuterio-3-phenylpropan-2-amine
Methylenedioxyhydroxyethylamphetamine
2-[4-(1-Phenylpropan-2-ylamino)butyl]isoindole-1,3-dione
N-Deuterio-1-phenylpropan-2-amine
N-(1-Phenylpropan-2-yl)-1,3-benzodioxol-5-amine
3-Fluoromethamphetamine
(2S)-2,6-Diamino-4,4,5,5-tetradeuterio-N-[(2S)-1-phenylpropan-2-yl]hexanamide; dihydrochloride
Butanediamide; (2S)-1-phenylpropan-2-amine
(2S)-2-Amino-5-(diaminomethylideneamino)pentanoic acid; 1-phenylpropan-2-amine; acetate
(4S)-4-[[2-[[(2S)-2-[[(2S)-2-[[(2R)-2-[[(2S)-2-[[(2S)-1-[[(2S)-2-[[(2S)-5-Amino-2-[[2-[[(2S)-2-amino-3-(4-
hydroxyphenyl)propanoyl]amino]acetyl]amino]-5-oxopentanoyl]amino]-3-methylbutanoyl]pyrrolidine-
2-carbonyl]amino]-4-methylsulfanylbutanoyl]amino]-3-sulfanylpropanoyl]amino]-
3-carboxypropanoyl]amino]propanoyl]amino]acetyl]amino]-5-[[(2S)-5-amino-1-[[(2R)-1-[[(2S)-1-
[[(1S)-1-carboxy-2-methylpropyl]amino]-1-oxopropan-2-yl]amino]-1-oxo-3-sulfanylpropan-2-yl]amino]-
1,5-dioxopentan-2-yl]amino]-5-oxopentanoic ac
4,4,4-Trideuterio-1-phenyl-3,3-bis(trideuteriomethyl)butan-2-amine
1-(4-Deuteriooxyphenyl)propan-2-amine
N-lodo-N-(1-phenylpropan-2-yl)butan-1-amine
Brolamfetamine hydrobromide
3,5-Dimethyl-4-methoxyamphetamine hydrochloride
2,4,6-Trimethylamphetamine hydrochloride
Ortetamine
L-Histidyl-N-(1-methyl-2-phenylethyl)-5-oxo-L-prolyl-L-prolinamide
Butanamide, 2,2,3,3,4,4,4-heptafluoro-N-(1-methyl-2-phenylethyl)-
(R)-3,4-Dimethoxyamphetamine
Methanol, [(1-methyl-2-phenylethyl)amino]-
CID 129318240
N-(Dimethoxymethyl)-N-methyl-1-phenylpropan-2-amine
2-[5-(2-Aminopropyl)-2-hydroxyphenyl]acetic acid
2-[4-(2-Aminopropyl)-2-hydroxyphenyl]ethane-1,1-diol
2,3,4,5,6-Pentachlorobenzenethiol; 1-phenylpropan-2-amine; 2-sulfanylidene-1H-pyridine-3-carboxylic acid
DL-Methamphetamine
5-Methoxy-3,4-methylenedioxyamphetamine
2,4,5-Trimethoxyamphetamine
2-(alpha-Methylphenethyl)aminoethanol
2-Methoxy-4,5-methylenedioxyamphetamine
2,3-Dimethoxyamphetamine hydrochloride
5-Methoxy-3,4-methylenedioxyamphetamine hydrochloride
2,5-Dimethoxy-3-nitroamphetamine
4-Imidazolepropionamide, N-(alpha-methylphenethyl)-alpha-((2-oxo-5-pyrrolidinyl)formamido)-
4-(2-(Ethylamino)propyl)-2-methoxyphenol
alpha,2,2-Trimethyl-1,3-benzodioxole-5-ethanamine
N-Methyl-N-propylamphetamine
Sulfuric acid; (2R)-1,1,1-trideuterio-3-phenylpropan-2-amine
(+)-N,N,alpha-Trimethylbenzeneethanamine hydrochloride
N,N-Difluoro-1-phenylpropan-2-amine
2-Fluoromethamphetamine
N-(1-Phenylpropan-2-yl)butan-1-amine; hydrochloride
CID 55254975
(2S)-N-Methyl-1-phenylpropan-2-amine; 1-phenylpropan-2-amine
1-(1,3-Benzodioxol-5-yl)-N-[tert-butyl(dimethyl)silyl]oxypropan-2-amine
N,N-Dideuterio-1-phenylpropan-2-amine
1-[3-(Deuteriomethoxy)phenyl]propan-2-amine
4-(2-Aminopropyl)-2-(2-hydroxyethyl)phenol
1-Phenylpropan-2-amine; 2-sulfanylidene-1H-pyridine-3-carboxylic acid
p-Methoxymethamphetamine
3,5-Dimethoxyamphetamine hydrochloride
3-Methylamphetamine
N,N-Dimethyl-3,4-methylenedioxyamphetamine
Silanamine, 1,1,1-trimethyl-N-(1-methyl-2-phenylethyl)-N-(trimethylsilyl)-, (+/−)-
N-Ethyl-alpha-methyl-1,3-benzodioxole-5-ethanamine hydrochloride
Methyl-DOB
(2S)-1-(3,4-Dimethoxyphenyl)propan-2-amine
[4-(2-Aminopropyl)phenyl]methanol
2-Deuterio-N-(1-phenylpropan-2-yl)acetamide
N-(1,2-Dimethoxy-3-phenylpropan-2-yl)-N-propan-2-ylaniline

In certain embodiments, the molecule are THC molecules or derivatives thereof. In certain embodiments, the molecule is selected from any of the molecules in the table below:

delta8-THC
11-Hydroxy-THC
11-Nor-9-carboxy-thc
11-Nor-9-carboxy-delta9-THC-D9
delta-9 Thc trifluoroacetate
delta(8)-Thc-11-oic acid
8beta-Hydroxy-dleta(9)-THC
ABN-delta8-THC
delta(Sup 8)-Thc sulfate potassium salt
THC, 11-OH-delta 8
8-Hydroxy-delta-9-THC
n-Hexyl-delta-9-thc
11-Hydroxy-delta(8)-thc
2′-Hydroxy-delta(9)-thc
Nor-thc oxime carboxymethyl ether
8beta, 11-Dihydroxy-delta(9)-thc
11-Oxo-thc
5′-Tma-thc
2-IA-8-Thc
3′-Hydroxy-thc
3′,11-Dihydroxy-thc
4′-Hydroxy-thc
Thc-11-oic acid glucuronide
11-Nor-delta-9-thc-9-aldehyde
THC-O-phosphate
(R,S)-Fmoc-Thc(Boc)-OH
Abn-delta(8)-thc
Thorium carbide (ThC)
Dronabinol
11-Nor-9-carboxy-delta-9-tetrahydrocannabinol
delta9-Tetrahydrocannabinol
delta8-Tetrahydrocannabinol
8-Hydroxy-delta(9)-tetrahydrocannabinol
11-Hydroxytetrahydrocannabinol
6H-Dibenzo(b,d)pyran-1-ol, 6a,7,8,10a-tetrahydro-6,6,9-trimethyl-3-pentyl-, (6aS-trans)-
Dimethyl-heptyl tetrahydrocannabinol
Unii-0Q7wyx30LC
Exo-Tetrahydrocannabinol
(R,R)-cis-Diethyl tetrahydro-2,8-chrysenediol
Dexanabinol
delta-9-Tetrahydrocannabinol dichloroethyl carbamoyl ester
3,4,5-Trihydroxy-6-(1-hydroxy-6,6-dimethyl-3-pentyl-6a,7,8,10a-tetrahydrobenzo[c]chromene-9-carbonyl)oxyoxane-2-carboxylic
acid
delta3-Tetrahydrocannabinol, (S)-
1-Hydroxy-6,6-dimethyl-3-(5,5,5-trideuteriopentyl)-6a,7,8,10a-tetrahydrobenzo[c]chromene-9-carboxylic acid
(6Ar,10aR)-3-(5-hydroxypentyl)-6,6,9-trimethyl-6a,7,8,10a-tetrahydrobenzo[c]chromen-1-ol
Tetrahydrocannabinol acetate
O-Methyl-delta-9 tetrahydrocannabinol
11-Hydroxy-delta8-tetrahydrocannabinol
6H-Dibenzo(b,d)pyran, 6a,7,10,10a-tetrahydro-1-methoxy-6,6,9-trimethyl-3-pentyl-, (6aR-trans)-
(7S)-6,6,9-Trimethyl-3-pentyl-6a,7,8,10a-tetrahydrobenzo[c]chromene-1,7-diol
delta3-Tetrahydrocannabinol, (R)-
delta(9)-Tetrahydrocannabinolic acid
3-Heptyl-delta(1)-tetrahydrocannabinol
(6Ar,10aR)-3-(3-ethylhexyl)-6,6-dimethyl-9-methylidene-7,8,10,10a-tetrahydro-6aH-benzo[c]chromen-1-ol
(6Ar,10aR)-3-(3-ethylhexyl)-1-methoxy-6,6-dimethyl-9-methylidene-7,8,10,10a-tetrahydro-6aH-benzo[c]chromene
(6Ar,10aR)-3-(3-hydroxypentyl)-6,6,9-trimethyl-6a,7,8,10a-tetrahydrobenzo[c]chromen-1-ol
Dimethylheptylpyran
6H-Dibenzo(b,d)pyran-1-ol, 6a,7,10,10a-tetrahydro-3,6,6,9-tetramethyl-
Propyl-delta(8)-tetrahydrocannabinol
Butyl-delta(8)-tetrahydrocannabinol
(6Ar,10aR)-1-methoxy-6,6-dimethyl-9-methylidene-3-pentyl-7,8,10,10a-tetrahydro-6aH-benzo[c]chromene
(6Ar,10aR)-6,6,9-trimethyl-3-(5,5,5-trifluoropentyl)-6a,7,8,10a-tetrahydrobenzo[c]chromen-1-ol
[(6Ar,10aR)-6,6-dimethyl-3-(2-methyloctan-2-yl)-1-[(4-phenylphenyl)methoxy]-6a,7,8,10a-tetrahydrobenzo[c]chromen-9-
yl]methanol
(6Ar,10aR)-3-(5-fluoropentyl)-6,6,9-trimethyl-6a,7,8,10a-tetrahydrobenzo[c]chromen-1-ol
5-[(6Ar,10aR)-1-hydroxy-9-(hydroxymethyl)-6,6-dimethyl-6a,7,8,10a-tetrahydrobenzo[c]chromen-3-yl]pentyl-trimethylazanium
2-[2-[[(6Ar,10aR)-6,6,9-trimethyl-3-pentyl-6a,7,8,10a-tetrahydrobenzo[c]chromen-1-y]]oxy]ethyl]morpholine
6,6,9-Trimethyl-3-(5,5,5-trideuteriopentyl)-6a,7,8,10a-tetrahydrobenzo[c]chromen-1-ol
Tetrahydrocurcumin
1′,2′-Dimethylheptyl-1-deoxy-delta(8)-tetrahydrocannabinol
6a,7,10,10a-Tetrahydro-1-hydroxy-6,6-dimethyl-3-pentyl-6H-dibenzo(b,d)pyran-9(8H)-one
Ethyl-delta-9-tetrahydrocannabinol
(S)-Tetrahydrocolumbamine
6H-Dibenzo[b,d]pyran-1,8-diol, 6a,7,8,9,10,10a-hexahydro-6,6-dimethyl-9-methylene-3-pentyl-
(6Ar,10aR)-3-(5-bromopentyl)-6,6,9-trimethyl-6a,7,8,10a-tetrahydrobenzo[c]chromen-1-ol
(6Ar,8S,10aR)-6,6,9-trimethyl-3-pentyl-6a,7,8,10a-tetrahydrobenzo[c]chromene-1,8-diol
Chalconaringenin 4′-glucoside
S-[2-[3-[[4-[[[(2R,3S,4R,5R)-5-(6-Aminopurin-9-yl)-4-hydroxy-3-phosphonooxyoxolan-2-yl]methoxy-hydroxyphosphoryl]oxy-
hydroxyphosphoryl]oxy-2-hydroxy-3,3-dimethylbutanoyl]amino]propanoylamino]ethyl] (2R,3S,6S)-3-fluoro-2-methyl-6-
[(3R,5S,7R,8R,9S,10S,12S,13R,14S,17R)-3,7,12-trihydroxy-10,13-dimethyl-2,3,4,5,6,7,8,9,11,12,14,15,16,17-tetradecahydro-
1H-cyclopenta[a]phenanthren-17-yl]heptanethioate
(R,S)-3-Boc-amino-9-boc-1,2,3,4-tetrahydro-carbazole-3-carboxylic acid
(6Ar,10aR)-6,6,9-trimethyl-3-[(2S,3R)-3-methyloctan-2-yl]-6a,7,10,10a-tetrahydrobenzo[c]chromen-1-ol
11-Nor-delta(9)-tetrahydrocannabinol-9-carboxylic acid
[(6Ar,10aR)-1-methoxy-6,6-dimethyl-3-(2-methyloctan-2-yl)-6a,7,8,10a-tetrahydrobenzo[c]chromen-9-yl]methanol
(6Ar,10aR)-3-(5-iodopentyl)-6,6,9-trimethyl-6a,7,8,10a-tetrahydrobenzo[c]chromen-1-ol
3-[[(6Ar,10aR)-6,6,9-trimethyl-3-pentyl-6a,7,8,10a-tetrahydrobenzo[c]chromen-1-yl]oxy]propan-1-amine
2-[4-[[(6Ar,10aR)-9-(fluoromethyl)-6,6-dimethyl-3-pentyl-6a,7,8,10a-tetrahydrobenzo[c]chromen-1-yl]oxy]butyl]indene-1,3-dione
(6Ar,10aR)-6,6,9-trimethyl-3-pentyl-6a,7,8,10a-tetrahydrobenzo[c]chromen-1-ol; 2-[(2-nitrophenyl) sulfanylamino]acetate
(6Ar,10aR)-6,6,9-trimethyl-3-pentyl-6a,7,8,10a-tetrahydrobenzo[c]chromen-1-ol; 3-aminopropanoate
(6,6-Dimethyl-9-methylidene-3-pentyl-7,8,10,10a-tetrahydro-6aH-benzo[c]chromen-1-yl)oxy-trimethylsilane
(2S,3S,4S,5R,6S)-3,4,5-Trihydroxy-6-[1-hydroxy-6,6-dimethyl-3-(5,5,5-trideuteriopentyl)-6a,7,8,10a-
tetrahydrobenzo[c]chromene-9-carbonyl]oxyoxane-2-carboxylic acid
CID 131728058
(6Ar,10aR)-6,6,9-trimethyl-3-pentyl-6a,7,8,10a-tetrahydrobenzo[c]chromen-1-ol; 2-[[2-[(2-methylpropan-2-
yl)oxycarbonylamino]acetyllamino]acetic acid
1-Methoxy-6,6,9-trimethyl-3-pentyl-6a,7,10,10a-tetrahydro-6H-benzo[c]chromene
(6Ar,10aR)-6,6,9-trimethyl-3-(5,5,5-trideuteriopentyl)-6a,7,8,10a-tetrahydrobenzo[c]chromen-1-ol
delta-9-cis-Tetrahydrocannabinol, (−)-
6,6-Dimethyl-9-methylidene-3-pentyl-7,8,10,10a-tetrahydro-6aH-benzo[c]chromen-1-ol
Trimethylsilyl 6,6-dimethyl-3-pentyl-1-trimethylsilyloxy-6a,7,8,10a-tetrahydrobenzo[c]chromene-9-carboxylate
[(6Ar,10aR)-6,6,9-trimethyl-3-pentyl-6a,7,8,10a-tetrahydrobenzo[c]chromen-1-yl] 4-methylbenzenesulfonate
(6Ar,10aR)-6,6-dimethyl-9-methylidene-3-(2-methyloctan-2-yl)-7,8,10,10a-tetrahydro-6aH-benzo[c]chromen-1-ol
[(6Ar,10aR)-6,6,9-trimethyl-3-pentyl-6a,7,8,10a-tetrahydrobenzo[c]chromen-1-yl] carbamate
(6As,10aS)-9-(hydroxymethyl)-6,6-dimethyl-3-(2-methyloctan-2-yl)-6a,7,8,10a-tetrahydrobenzo[c]chromen-1-ol
6-[[(6Ar,10aR)-6,6,9-trimethyl-3-pentyl-6a,7,8,10a-tetrahydrobenzo[c]chromen-1-yl]oxy]hexan-1-amine
(6Ar,10aR)-6,6,9-trimethyl-3-pentyl-6a,7,8,10a-tetrahydrobenzo[c]chromen-1-ol; 2-aminoacetate
(6Ar,10aR)-6,6,9-trimethyl-3-pentyl-6a,7,8,10a-tetrahydrobenzo[c]chromen-1-ol; 3-[(2-nitrophenyl)sulfanylamino]propanoate
(6Ar,10aR)-3-(4-hydroxypentyl)-6,6,9-trimethyl-6a,7,8,10a-tetrahydrobenzo[c]chromen-1-ol
(6Ar,10aR)-1-hydroxy-6,6-bis(trideuteriomethyl)-3-(5,5,5-trideuteriopentyl)-6a,7,8,10a-tetrahydrobenzo[c]chromene-9-carboxylic
acid
Butyl-delta(9)-tetrahydrocannabinol
3alpha, 7alpha, 12alpha-Trihydroxy-24-oxo-5beta-cholestan-26-oyl-CoA
(6Ar,10aR)-2-iodo-6,6-dimethyl-9-methylidene-3-pentyl-7,8,10,10a-tetrahydro-6aH-benzo[c]chromen-1-ol
(6Ar,10aR)-6,6,9-trimethyl-3-pentyl-1-propan-2-yloxy-6a,7,8,10a-tetrahydrobenzo[c]chromene
9-(Hydroxymethyl)-6,6-dimethyl-3-(5,5,5-trideuteriopentyl)-6a,7,8,10a-tetrahydrobenzo[c]chromen-1-ol
(6Ar)-6,6-dimethyl-9-methylidene-3-pentyl-7,8,10,10a-tetrahydro-6aH-benzo[c]chromen-1-ol
Benzo[c]chromene, 9-formyl-1-hydroxy-3-pentyl-6,6-dimethyl-6a,7,10,10a(6H)-tetrahydro-
(+)-6,6,9-Trimethyl-3-pentyl-6a,7,8,10a-tetrahydro-6H-benzo[c]chromen-1-ol
Ethyl-delta-8-tetrahydrocannabinol
6-[(6Ar,10aR)-1-hydroxy-6,6-dimethyl-3-pentyl-6a,7,8,10a-tetrahydrobenzo[c]chromene-9-carbonyl]oxy-3,4,5-trihydroxyoxane-2-
carboxylic acid
delta1-Tetrahydrocannabiorcol
3,7,12-Trihydroxycholestan-26-oyl-CoA
(6Ar)-9-(hydroxymethyl)-6,6-dimethyl-3-(2-methyloctan-2-yl)-6a,7,8,10a-tetrahydrobenzo[c]chromen-1-ol
Unii-8hhx5B1WP5
1-Methoxy-6,6,9-trimethyl-3-pentyl-6a,7,8,10a-tetrahydrobenzo[c]chromene
11-Acetoxy-delta(8)-tetrahydrocannabinol
(6Ar,10aR)-1-hydroxy-6,6-dimethyl-3-(2-methyloctan-2-yl)-6a,7,8,10a-tetrahydrobenzo[c]chromene-9-carboxylic acid
4-[[(6Ar,10aR)-6,6,9-trimethyl-3-pentyl-6a,7,8,10a-tetrahydrobenzo[c]chromen-1-yl]oxy]butan-1-amine
(6Ar,10aS)-10-(hydroxymethyl)-6,6,9-trimethyl-3-pentyl-6a,7,8,10a-tetrahydrobenzo[c]chromen-1-ol
(6Ar,10aR)-6,6-dimethyl-9-methylidene-3-pentyl-1-(4-phenylphenoxy)-7,8,10,10a-tetrahydro-6aH-benzo[c]chromene
4-[(6Ar,10aR)-1-methoxy-6,6,9-trimethyl-3-pentyl-6a,7,8,10a-tetrahydrobenzo[c]chromen-8-yl]morpholine
Unii-910865dji7
(1″,2″-Dimethyl-5″-ethyl)-delta6-tetrahydrocannabinol
(6Ar,10aR)-3-(2-hydroxypentyl)-6,6,9-trimethyl-6a,7,8,10a-tetrahydrobenzo[c]chromen-1-ol
Tetrahydrocannabivarin
6H-Dibenzo(b,d)pyran-1-ol, 3-hexyl-6a,7,10,10a-tetrahydro-6,6,9-trimethyl-, (6aR-trans)-
(6Ar,10aR)-9-(fluoromethyl)-1-methoxy-6,6-dimethyl-3-pentyl-6a,7,8,10a-tetrahydrobenzo[c]chromene
(6,6,9-Trimethyl-3-pentyl-6a,7,10,10a-tetrahydrobenzo[c]chromen-1-yl) 2,2,2-trifluoroacetate
6H-Dibenzo(b,d)pyran-1-ol, 6a-beta,7,8,10-a-beta-tetrahydro-3-pentyl-6,6,9-trimethyl-, (+−)-Z-
Ajulemic acid
(6AR,10AR)-3-(1,1-Dimethylbutyl)-6A,7,10,10A-tetrahydro-6,6,9-trimethyl-6H-dibenzo[B,D]pyran
3-(5-Fluoropentyl)-6,6,9-trimethyl-6a,7,10,10a-tetrahydrobenzo[c]chromen-1-ol
6,6,9-Trimethyl-3-(5,5,5-trifluoropentyl)-6a,7,10,10a-tetrahydrobenzo[c]chromen-1-ol

In certain embodiments the molecule is curcuphenol or derivatives thereof. In certain embodiments, the molecule is selected from any of the molecules in the table below:

Curcuphenol
Phenol, 2-((1S)-1,5-dimethyl-4-hexenyl)-5-methyl-
5-Methyl-2-[(2R)-6-methylhept-5-en-2-yl]phenol
(3S,5S,6S)-2-(Hydroxymethyl)-6-[5-methyl-2-[(2S)-6-methylhept-5-en-2-yl]phenoxyloxane-3,4,5-triol
[5-Methyl-2-[(2R)-6-methylhept-5-en-2-yl]phenyl] acetate
Hydroxy-[4-hydroxy-2-methyl-5-[(2S)-6-methylhept-5-en-2-yl]phenyl]-oxoazanium
2-[(1S)-1,5-Dimethylhex-4-enyl]-5-(hydroxymethyl)phenol
5-Methyl-2-[(2s)-6-methylhept-5-en-2-yl]phenyl pyridine-4-carboxylate
2-[(S)-6-Hydroxy-1,5-dimethyl-4-hexenyl]-5-methylphenol
2-[(1S,4E)-6-Hydroxy-1,5-dimethylhex-4-enyl]-5-(hydroxymethyl)phenol

In certain embodiments, the ABCF1 agonist/inducer is a natural product. In certain embodiments, the ABCF1 agonist/inducer is from an Ascomycetes fungus. In specific embodiments the ABCF1 agonist/inducer is from Cordyceps sinensis and related fungi. The Cordyceps sinensis product may be fresh, dried or an extract. In certain embodiments, the natural product is from a mushroom. Exemplary mushroom genera include Agrocybe, Amanita, Conocybe, Galerina, Gymnopilus, Hypholoma, Inocybe, Panaeolus, Psilocybe, Pholiotina, Pluteus and Weraroa.

ABCF1 agonist may be used alone or in combination with one or more other therapeutics.

ABCF1 may be used as a biomarker. Methods of measuring gene expression including mRNA and protein expression are known in the art. In certain embodiments, decreased expression of ABCF1 is indicative of an inflammatory and/or immune response. In certain embodiments, increased expression of ABCF1 is indicative of a decreased inflammatory and/or immune response. Accordingly, ABCF1 expression may be used as a biomarker for diseases or disorders associated with increased or decreased inflammatory and/or immune responses. ABCF1 expression may be used in methods of determining clinical outcome of diseases and/or disorders associated with increased or decreased inflammatory and/or immune responses. Accordingly, in certain embodiments, the present invention provides a method of determining clinical outcome of diseases and/or disorders associated with increased or decreased inflammatory and/or immune responses by determining expression of one or more genes including Abcf1.

ABCF1 may be used as a biomarker for inflammation and/or immune response associated with autoimmune diseases and/or neuroinflammatory diseases. In certain embodiments, ABCF1 may also be used as a biomarker for MDD. In certain embodiments, the present invention provides a method of determining clinical outcome of a patient with MDD by determining expression of one or more genes including Abcf1. The patient may have comorbidities such as autoimmune diseases.

Accordingly, in certain embodiments, the present invention provides a method of determining clinical outcome of an autoimmune patient with comorbid MDD by determining expression of one or more genes including Abcf1.

In certain embodiments, there is provided bioassay screens which utilize ABCF1 to identify new drugs for treatment of MDD. In certain embodiments, there is provided bioassay screens which utilize ABCF1 to identify new drugs for treating autoimmune and comorbid neuropsychiatric disorders. For example, the screens may be used to identify drugs that modulate an immune response.

In certain embodiments, there is provided methods to determine ABCF1 expression. Such methods may be used to identify agents that modulate ABCF1 expression and therefore may be useful in the identification of drugs. In specific embodiments, a reporter gene is placed under the control of the ABCF1 promoter and the reporter gene product is measured (either qualitatively or quantitatively). Cells, including but not limited macrophages such as RAW 264.7 cell line, comprising the ABCF1 promoter reporter gene product may be used in assays to identify agents that modulate ABCF1 expression.

To gain a better understanding of the invention described herein, the following examples are set forth. It will be understood that these examples are intended to describe illustrative embodiments of the invention and are not intended to limit the scope of the invention in any way.

### Example 1

### A. Background, Significance and Impact of Research:

Association of depression with chronic inflammation: Major depressive disorder (MDD), often referred to as “depression’, affects psychosocial functioning and diminishes the quality of life¹. It affects over 300 million people worldwide² and is associated with ˜800,000 suicide deaths annually³. The World Health Organization states that MDD will become the third most prevalent disease in the World by 2030⁴. It occurs in higher prevalence in women than in men, but the aetiology of depression remains poorly understood. It appears to be caused by both genetic and environmental factors, however, its diagnosis and management are clinically challenging because of unpredictable presentation and response to treatment⁴. Furthermore, depression remains associated with premature mortality from suicide and other illnesses⁵. A traditional hypothesis is that those living with depression have a deficiency in monoamine neurotransmitters such as serotonin and norepinephrine in the brain, however, evidence now shows that some forms of depression are associated with ongoing forms of low-grade inflammation⁶.

This is a surprising connection, transforming conventional thinking: Subsets of depression patients have an impaired peripheral immune system, increased levels of proinflammatory cytokines that can affect neurotransmitter metabolism, neuroendocrine function and regional brain activity⁷. Patients given proinflammatory cytokines, such as IL-1b, experience more symptoms of anxiety and depression than untreated patients⁷, and patients experiencing bacterial and viral infections often experience symptoms associated with depression (i.e. disrupted sleep, fatigue, depressed moods, impaired concentration)⁸. Studies link MDD to higher levels of inflammatory markers compared to those who are not clinically depressed. A study of >14,000 patients showed those with depression had 46% higher levels of C-reactive protein (CRP), an inflammation marker, in their blood⁸.

The Role of Innate and Adaptive Immune Systems Alterations in the Pathophysiology and Treatment of Depression: The immune balance between Th1/Th2 and Th17/Treg correlate with MDD¹⁷. Depressed subjects have an increase in peripheral Th17 cell number and a decrease in T-reg cell number resulting in imbalance of Th17/Treg ratio compared to healthy controls¹⁸. Furthermore, studies show that pregnant patients with MDD have elevated inflammatory responses^{19,20} and higher levels of circulating steroids compared to healthy pregnant women²¹. Specifically women exhibiting severe depression (SD) and severe anxiety (SA) during pregnancy exhibit high levels of Th1-(IL-6, TNF-α, IL-2, IFN-γ), Th17-(IL-17A, IL-22), and Th2-(IL-9, IL-10, and IL-13) related cytokines. The SA group alone showed higher concentrations of Th1-(IL-6, TNF-α, IL-2, IFN-γ) and Th2-(IL-4, and IL-10) cytokines versus the controls¹⁷.

Moreover, the immune balance between M1/M2 macrophages has previously been proposed as a target of therapy for MDD²². Studies on humans and animals have documented that chronic activation of M1 microglial cells²³⁻²⁵ may trigger mood disorders²⁶ through the release of a variety of chemokines, eicosanoids, free radicals, neurotoxins, pro-inflammatory cytokines, and nitric oxide²³, thereby potentiating neuronal dysfunction²⁷. Various bacterial and viral infections including influenza virus, Herpes viruses, and HIV induce the secretion of proinflammatory cytokines and induce microglial activation that is associated with depression symptoms²⁸⁻³². Experimental induction in humans with immune activators that activate microglia such as endotoxin (LPS) a key driver of SIRS or gram-negative bacteria such as Salmonella typhimurium induces depressive symptoms, where the severity is correlated with elevated blood levels of inflammatory cytokines³³⁻³⁵. In animal models, LPS administration induces microglial activation together with depression symptoms in rodents that is halted with selective serotonin reuptake inhibitors (SSRIs) or tricyclic antidepressants (TCAs)^{36,37}. In fact, many observations support the involvement of microglia in LPS-induced depression: (i) LPS-induced depression symptoms can be reduced by treatment with the microglial inhibitor minocycline 38; (ii) activation of the enzyme indoleamine 2,3-dioxygenase (IDO) in microglia is essential for the development of depression symptoms and microglial activation induced by LPS³⁸⁻⁴¹; and (iii) mice with microglial hyper-reactivity by traumatic brain injury⁴², or induced by a microglia-specific mutation in the fractalkine receptor⁴³ exhibit heightened LPS-induced depression symptoms. In contrast, mice deficient in NLRP3 inflammasome signalling resulting in induction of pro-inflammatory cytokine secretion have attenuated depression in response to LPS⁴⁴.

ABCF1-A missing link in inflammatory disease and depression: ABCF1 is a protein within the ABC (ATP-binding cassette) gene, family. Unlike other ABC family members, ABCF1 lacks the transmembrane domain and does not appear to function as a transporter. The ABCF1 gene is located in the class I region of the major histocompatibility complex locus on chromosome 6 in humans and on chromosome 17 in mice. Previous studies have shown that ABCF1 participates in translation initiation through its association with eIF2 and ribosomes⁴⁵⁻⁴⁹. ABCF1 is known to be located in the cytoplasm and nucleoplasm, but not in the nucleolus⁴⁷. Gene expression of ABCF1 has been shown to be elevated substantially in human synoviocytes isolated from the inflamed joints of rheumatoid arthritis patients, and this increases further when stimulated with TNF-α⁵⁰. Also, the ABCF1 locus is linked to increased susceptibility to autoimmune pancreatitis in the Japanese population⁵¹ and, importantly, ABCF1 has been associated with susceptibility to rheumatoid arthritis in European and Asian populations⁵². Immunological studies in mouse embryonic fibroblasts have shown that ABCF1 associates with dsDNA and DNA sensing components HMGB1 and IFI204, and further interacts with SET complex members (SET, ANP32A and HMGB2) to facilitate cytosolic DNA sensing mechanisms.

ABCF1 acts as a ubiquitin-switch that regulates inflammatory pathways: Although ABCF1 (+/) mice appear normal under specific pathogen-free conditions, we recently discovered that ABCF1 acts as a molecular switch between inflammatory pathways downstream of TLRs⁵³. In the Immunity paper, “The ATP-Binding Cassette Gene ABCF1 Functions as an E2 Ubiquitin-Conjugating Enzyme Controlling Macrophage Polarization to Dampen Lethal Septic Shock” (2019)⁵³, sepsis was studied, where little was known regarding the molecular switches and pathways that regulate this disease. It was discovered that ABCF1 possesses an E2 ubiquitin enzyme activity, through which it controls the LPS-Toll-like Receptor-4 (TLR4)-mediated gram-negative insult by targeting key proteins for K63-polyubiquitination. K63-ubiquitination by ABCF1 shifts the inflammatory profile from an early phase MyD88-dependent to a late phase TRIF-dependent signalling pathway, thereby regulating TLR4 endocytosis and modulating macrophage polarization from M1 to M2 phase. Physiologically, ABCF1 regulates the shift from the inflammatory phase of sepsis to the endotoxin tolerance phase and modulates cytokine storm and interferon-β-dependent production by the immunotherapeutic mediator, SIRT1. Consequently, ABCF1 controls sepsis-induced mortality by repressing hypotension induced renal circulatory dysfunction. Further, ABCF1 is necessary to maintain macrophage polarization in M2b state and the lack of ABCF1 shifts the state to the pro-inflammatory M1 state 53.

The molecular details of the ABCF1 switch: In the MyD88 pathway (M1 macrophage-like), the early phase of TLR4 signalling leads to UBC13 targeting TRAF6 for K63-polyubiquitination, which further targets cIAP1/2 for K63-polyubiquitination. cIAP1/2 then enhances K48-proteasomal degradation of ABCF1 and TRAF3. In the absence of ABCF1, TAK1 is phosphorylated, which leads to activation of MAPK and NF-kB pathways and elevated production of pro-inflammatory cytokines like TNFa, IL-1b, IL-6, thereby polarizing macrophages to M1 phenotype. Subsequently in the TRIF pathway (M2 macrophage-like), self K48-proteasomal degradation of cIAP1/2 results in K63-polyubiquitination of ABCF1 by TRAF6, which results in ABCF1 to bind and forms a complex with TRAF3 and SYK leading to the formation of K63-polyubiquitylated TRAF3 and SYK. This leads to TLR4 endocytosis into the endosomes, which then initiates TRIF-dependent TLR4 signalling and eventual production of IFN-I stimulated genes. This triggers phosphorylation of TBK1 that leads to phosphorylation and eventual dimerization of IRF3 and production of IFN-I stimulated genes. This shift from MyD88 to TRIF signalling by ABCF1 leads to increased production of IL-10, minimal production of TNFα, IL-1b, IL-6 and CD86, MHC-II surface markers and decreased CD206 levels, thus polarizing macrophages to M2b phenotype.

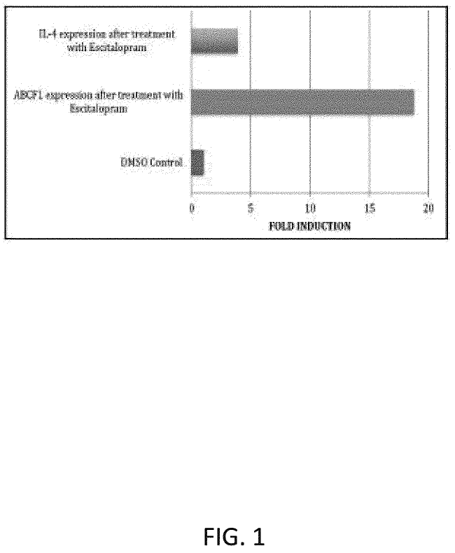

Surprising Connection, Transforming Conventional Thinking: Escitalopram is a selective serotonin reuptake inhibitor (SSRI) and has the highest selectivity for the serotonin transporter compared to the norepinephrine transporter, making the side-effect profile relatively mild in comparison to less selective SSRIs⁵⁴. Additionally, noradrenergic or serotonin-norepinephrine reuptake inhibitors used to treat major depressive disorder have anti-inflammatory properties in vitro⁵⁵. It is fascinating to contemplate that antidepressants, such escitalopram, appear to possess anti-inflammatory properties^{56,57,58-60}. Mechanistically, antidepressants likely mediate this through a reduction in pro-inflammatory cytokines IL-1b, TNFα, and IL-6 with a reciprocal increase in anti-inflammatory cytokines including IL-10α. Studies have also shown that single nucleotide polymorphisms in IL-6 and IL-11, and mRNA levels of TNFα, are predictive of clinical response to the SSRI, escitalopram^{63,64}. Also, escitalopram modulates mRNA levels of cytokines in mouse brain⁶⁵ and decreases cytokine mRNA levels in the circulating immune cells of depression patients⁶⁰. Furthermore, IL-6 mRNA levels correlate to clinical response in depressed patients treated with antidepressants⁶⁰, and several cytokines, including IL-1b and TNFα, acutely stimulate serotonin transporter activity in neurons. The alteration of transport activity in serotonergic neurons in the brain provides a mechanism by which cytokines can modulate serotonergic signalling, and subsequently influence emotional cognitive processing. Experimental induction in humans with immune activators, such as LPS that activate macrophages and microglia, act as key drivers of depression and reveal that the severity of depressive symptoms is correlated with elevated blood levels of pro-inflammatory cytokines³³⁻³⁵. Recently, ABCF1 was identified as a putative therapeutic target of escitalopram⁶⁶. In conjunction with the Genome-Based Therapeutic Drugs for Depression Project, published with the title “ABCF1 is identified as a putative therapeutic target of escitalopram in the inflammatory cytokine pathway,” the authors found that the peripheral blood mononuclear cells (PBMCs) of patients responding to escitalopram treatment subsequently increased the transcription of a single gene: ABCF1⁶⁶. Therefore, the effectiveness of a commonly used selective serotonin reuptake inhibitor correlates with ABCF1 expression. Furthermore, to confirm the observation that ABCF1 is elevated in the PBMCs of MDD patients is observable in vitro as well we find, in unpublished studies, that escitalopram induces IL-4 by approximately 5 fold and ABCF1 by approximately 20-fold in the macrophage cell line consistent with polarization towards an M2 phenotype (FIG. 1). Thus, the potential of ABCF1 as a therapeutic target is tantalising as it appears to reside at the intersection between inflammatory diseases and psychiatric illness.

These inventions will not only facilitate genomic and molecular biologic investigations on MDD and its link to inflammation, but this will also guide future development of antidepressant drugs. The strength of this invention is in the creation of a paradigm-changing interdisciplinary approach that will catalyze to initiate a novel direction of research, defying previous limitations in the literature 66.

### Example 2: The Effect of Psylocibin, Psylocin and their Analogs on ABCF1 Transcription

### Preparation of Cells:

1. Macrophage cell line RAW264.7 (ATCC) were grown to 80% confluency in growth media (DMEM+10% FBS+glutamine).
2. Dilutions of the drugs were made at desired final concentrations for a Dose response experiment. The concentrations' used for this experiment are: 10 nM, 100 nM, 500 nM for Psilocin, Psylocibin, 4-Acetoxy-N, N-dimthyltryptamine, O-Acetyl Psilocin Fumerate, and 4-acetoxyindole.
3. A time course response experiment was done for the above stated drugs at concentrations mentioned above (10 nM, 100 nM, 500 nM). The time points chosen were 0, 30 mins, 2 hours and 24 hour.
4. Untreated cells were used as negative control and Escitalopram at 0.3 mM was used as a positive control to activate ABCF1 expression for all the experiments.
   Analysis by qPCR:

Primers used:
GAPDH FP:
TGGATTTGGACGCATTGGTC
GAPDH RP:
TTTGCACTGGTACGTGTTGAT
ABCF1 FR:
AGAAAGCCCGAGTTGTGTTTG
ABCF1 PR:
GCCCCCTTGTAGTCGTTGATG

1. Post treatment with drugs at different time points, the reaction was stopped by removing the media with the drug. Cells were then collected and RNA was isolated from these.
2. After checking the quality of the RNA, cDNA was generated and qPCR was run with ABCF1 primers as the target gene and GAPDH as the house keeping gene.
3. Normalized against the expression level of GAPDH, the fold change expression level of ABCF1 was calculated and tabulated for all treatment conditions.

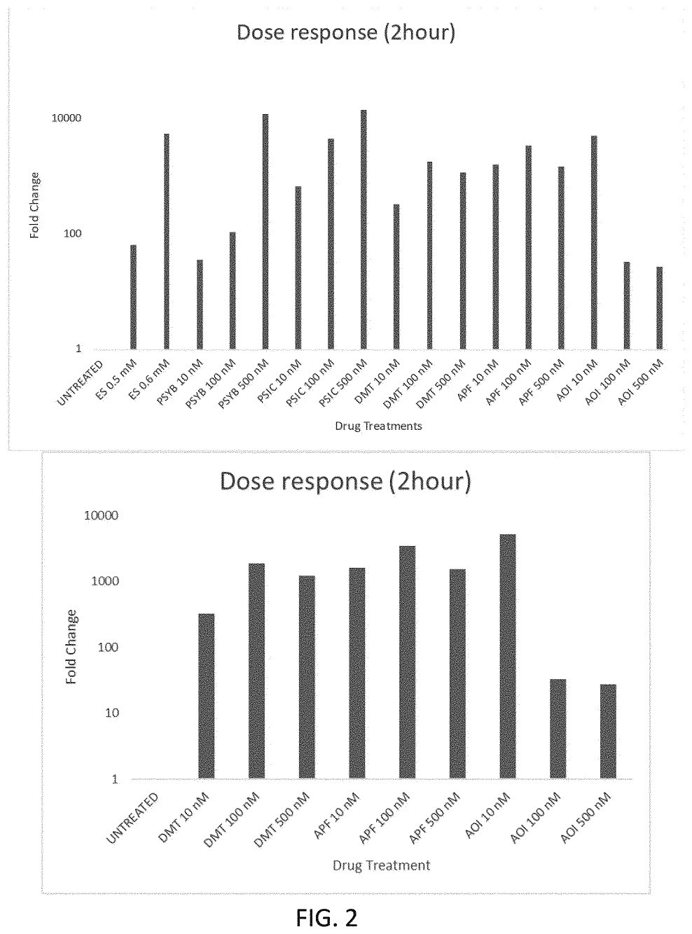

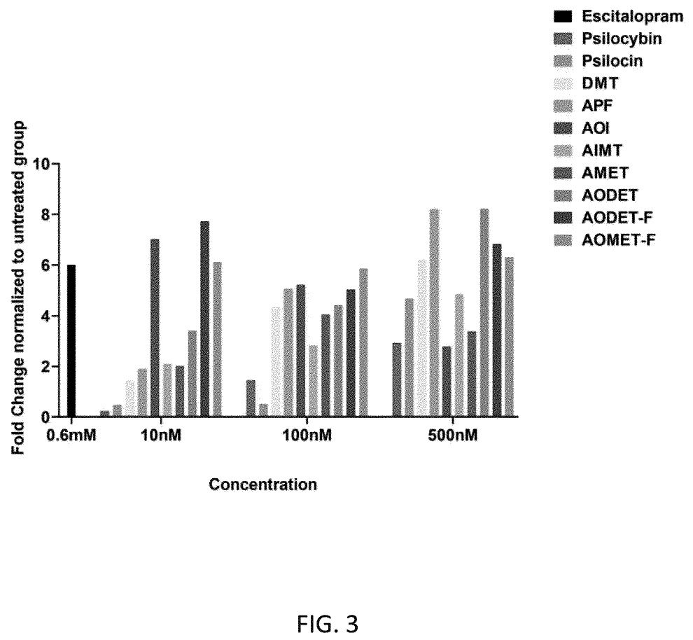

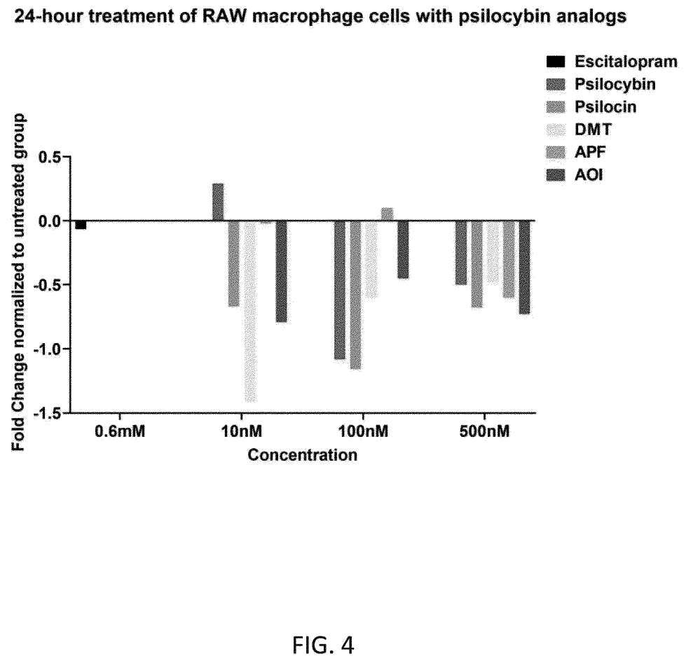

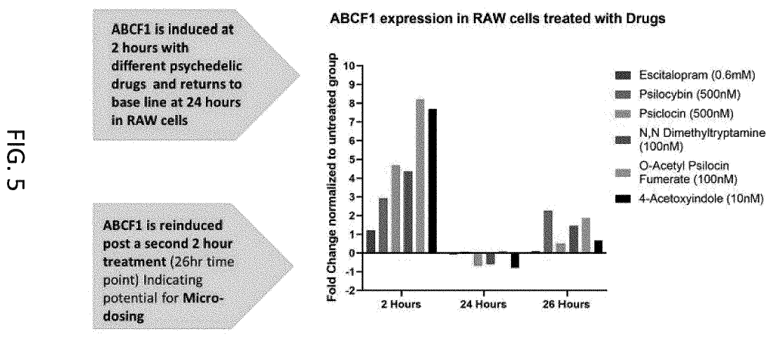

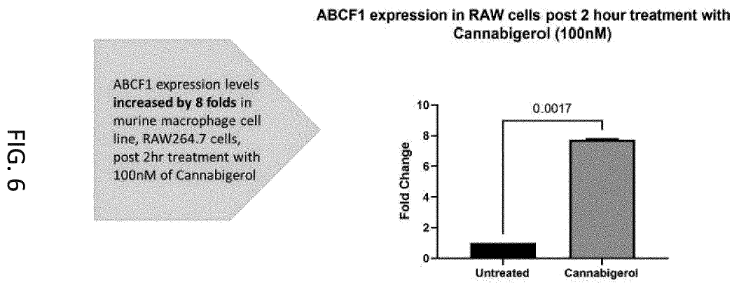

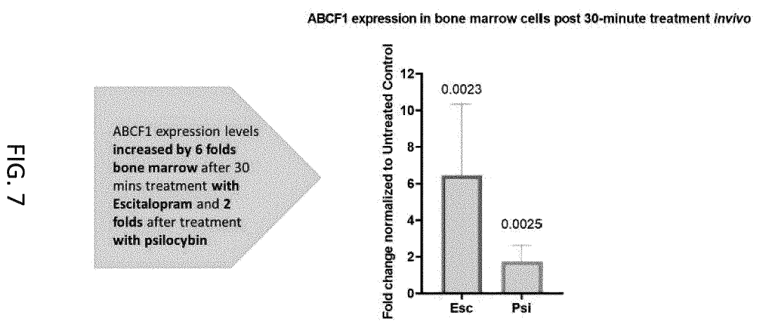

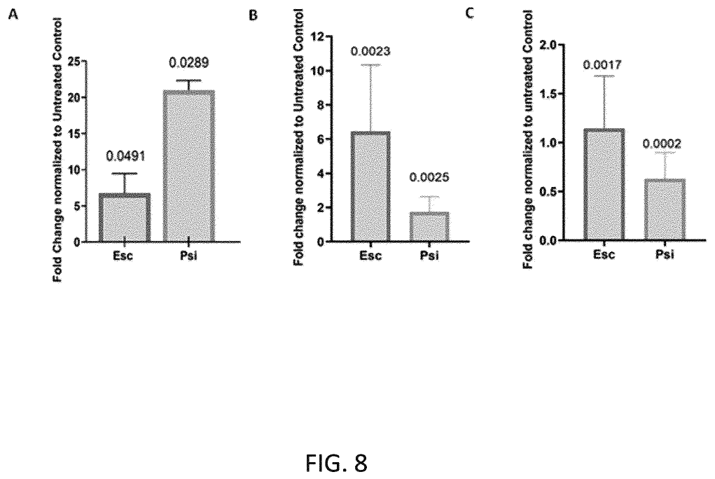

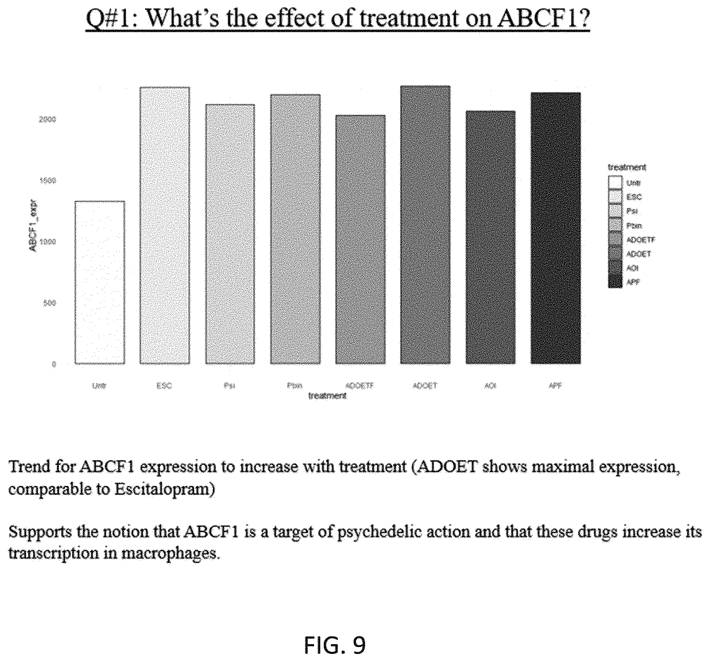

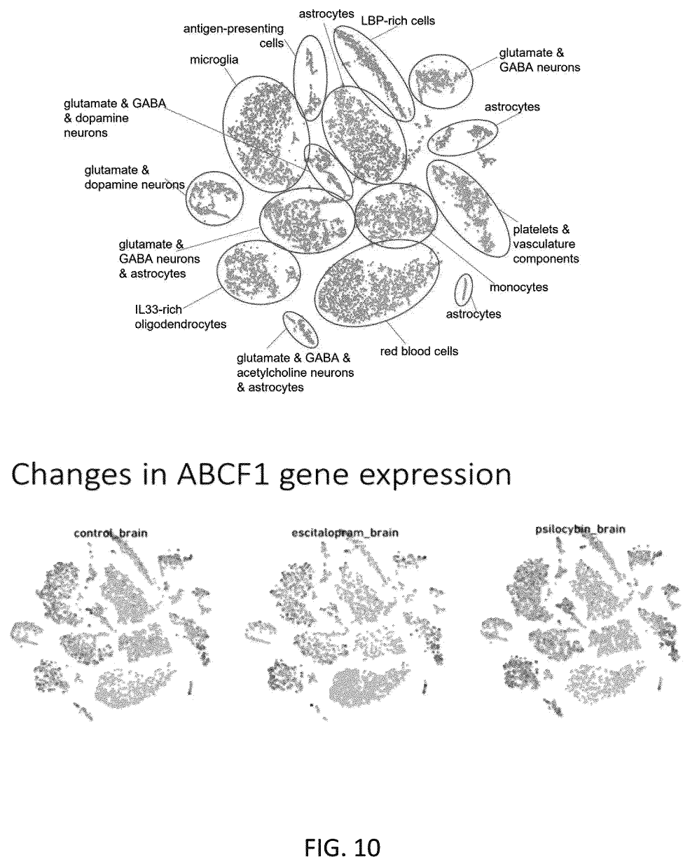

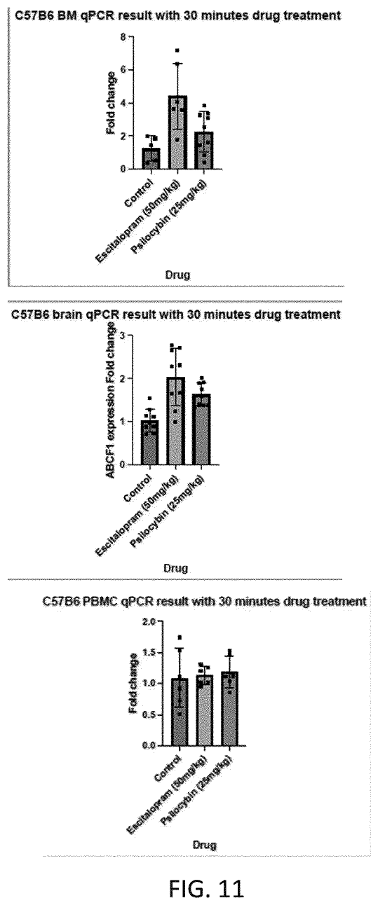

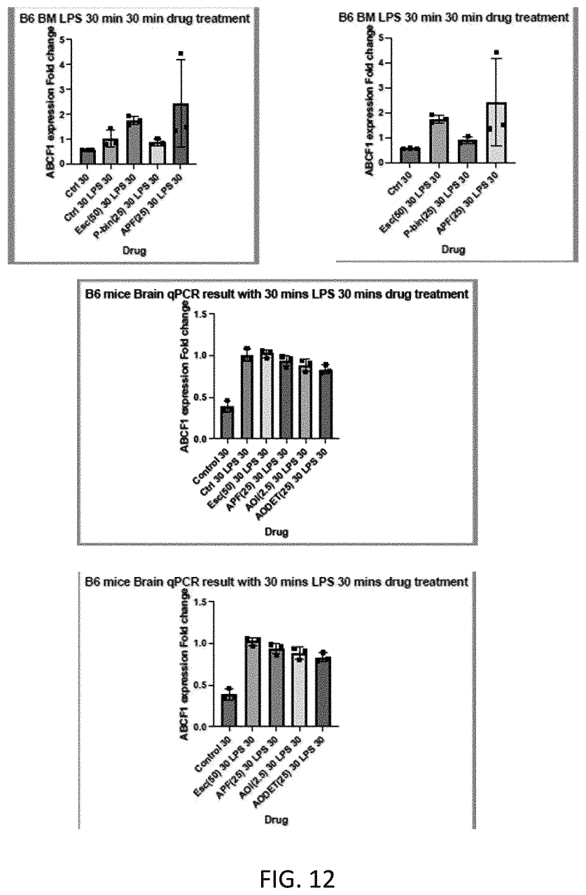

The results are shown in FIG. 2.

### Example 3: Changes in ABCF1 Expression in Bone Marrow, Brain and PBMCs Following Drug Treatment

### Mice Drug Treatment Protocol

Species strain: C57Bl/6
Tissues collected: Brain, Bone marrow, PBMC
Drug treatment:
   a. 30 minutes Drug treatment (PBS control, Escitalopram 50 mg/kg, Psilocybin 25 mg/kg)
   b. 30 minutes Drug treatment after 30 minutes LPS stimulation (PBS control, Escitalopram 50 mg/kg, O-Acetyl Psilocin Fumarate (APF) 25 mg/kg, 4-AcetoxyIndole (AOI) 2.5 mg/kg, 4-AcO-DET (AODET) 25 mg/kg)
Method:
   Weighed and injected the drug into the mice by Intraperitoneal injection.
   Euthanized the mice 30 minutes after injections with isoflurane and CO₂
   Post cervical Dislocation performed cardiac puncture to collect the whole blood from mice.
   Opened the cephalic bone to collect the whole brain.
   Removed the skin and muscles, took out both femur and tibia for bone marrow collection.
Tissue process procedures:
Brain—flash frozen and stored hemisphere in-80 for further molecular experiments and sent another part with buffer for sequencing. The frozen Brains were Dounce-homogenized. RNA was extracted from the cell suspension for qPCR analysis.
Bone Marrow
   cut a bit for both sides to open the femur and tibia, centrifuge out the bone marrow with 15000 rpm, 30 sec
   incubate 10 minutes in room temperature with 1 ml ACK buffer in 15 ml tube to lysis red blood cell.
   Dilute the lysed BM with 10 ml PBS immediately and centrifuge in room temperature, 5 minutes, 400 rpf
   Remove the supernatant
   Resuspend the pellet with 1 ml PBS and add 10 ml PBS to wash
   centrifuge in room temperature, 5 minutes, 250 rpf
   Remove the supernatant
   Resuspend the pellet with 1 ml PBS and put it into 1.5 ml Eppendorf tube
   centrifuge in room temperature, 5 minutes, 250 rpf
   Resuspend the pellet with 1 ml PBS
   Send half amount for sequencing and store other in pellet in-80 for further molecular experiments
PBMC
   Incubate the whole blood 10 minutes in room temperature with 5 ml ACK buffer in 50 ml tube to lysis red blood cell.
   Dilute the lysed PBMC with 45 ml PBS immediately and centrifuge in room temperature, 5 minutes, 400 rpf
   Remove the supernatant
   Resuspend the pellet with 1 ml PBS and add 49 ml PBS to wash
   centrifuge in room temperature, 5 minutes, 250 rpf
   Remove the supernatant
   Resuspend the pellet with 1 ml PBS and put it into 1.5 ml Eppendorf tube
   centrifuge in room temperature, 5 minutes, 250 rpf
   Resuspend the pellet with 1 ml PBS
   Send them all for sequencing

### Results

FIGS. 14 and 15 illustrate changes in ABCF1 expression following treatment.

### Example 3

Psilocybin, the active component of psychedelic “magic” mushrooms, has demonstrated effectiveness for treatment-resistant depression in several clinical trials by producing fast and long-lasting antidepressant effects.

The clinical implementation of psychedelic-assisted psychotherapy is difficult due to each patient requiring an individual psychedelic session (usually 6-8 hours) in the presence of a minimum of two trained clinical professionals, followed by psychotherapy integration sessions. As a result, personalized or precision medicine approaches are urgently needed to screen patients at baseline for their predicted response to psilocybin and to maximize the clinical feasibility of these novel antidepressant treatments.

Clinical evidence to inform the development of personalised approaches to psychedelic treatment will be obtained by assessing the safety profile and the efficacy of psilocybin-assisted psychotherapy in a randomized clinical trial (RCT); characterizing the molecular effects of a single acute dose of psilocybin on peripheral (blood) markers; identifying quantitative biomarkers that can predict antidepressant responses to psilocybin-assisted psychotherapy and identifying quantitative biomarkers that can predict potential adverse reactions to psilocybin.

### Clinical Trial Design

A double-blind, randomized, placebo-controlled clinical trial across a number of clinics in Canada. Forty individuals (both sexes) will be selected based on inclusion/exclusion criteria.

Before the clinical trial, applicants that scored 24 or higher in the Montgomery-Åsberg Depression Rating Scale (MADRS) will be screened for major depression disorder using the Mini-International Neuropsychiatric Interview (MINI) version 6.1. These participants will be randomized in either control or treatment arms, consisting of low- and high-dose psilocybin, respectively. Participants will be randomised to the treatment conditions by a professional biostatistician employing random non-stratified permuted blocks of varying length. Both clinicians and researchers will be blinded to block sizes.

The treatment arm (n=20) consists of treatment with a high psilocybin dose (30 mg/70 kg), while the control arm (n=20) consists of a low psilocybin dose (1 mg/70 kg, used as inactive placebo). Both doses will be administered orally as identical gelatine capsules. The psychedelics sessions will last approximately 6 hours and will take place in the presence of a minimum of two (preferably three) clinical psychologists and/or psychiatrists, of which at least one trained in psychotherapy, to monitor and provide emotional guidance and support to the participant. Physiological parameters (blood pressure, heart rate) will be recorded periodically during the trial. For the duration of the session, the participants will lie down on a couch and eye masks and headphones (playing a curated playlist of music for psychedelic therapy) will be provided, according to standard guidelines in place for psychedelic-assisted psychotherapy. For each participant, three in-person 1-hour psychotherapy sessions with a trained psychotherapist will be offered. These will take place three days before, as well as one day and two weeks after the psychedelic session.

Peripheral blood samples and clinical measures of depression will be collected at screening (baseline) and one day and two weeks after the psychedelic session.

The proposed RCT will consist of a treatment arm, receiving a high dose of psilocybin (30 mg/70 kg), and of a control arm, receiving a low dose of psilocybin (1 mg/70 kg). These doses were selected based on the following observations:

Evidence exists that 30-35 mg/70 kg is the optimal dose for antidepressant effects [Li, N.-X., et al., Journal of Affective Disorders, 2022. 296: p. 26-34.].

While high doses of psilocybin are more likely to induce psychologically challenging experiences [Griffiths, R. R., et al., Psilocybin occasioned mystical-type experiences: immediate and persisting dose-related effects. Psychopharmacology (Berl), 2011. 218 (4): p. 649-65.], we have strict exclusion criteria and monitoring procedures in place both before and during the trial to ensure that the incidence of adverse effects is minimized.

Evidence exists that a dose of 1 mg/70 kg is a suitable inactive placebo, given that subjective effects have been reported in some participants administered a dose as low as 3 mg/70 kg [Griffiths, R. R., et al., Psilocybin occasioned mystical-type experiences: immediate and persisting dose-related effects. Psychopharmacology (Berl), 2011. 218 (4): p. 649-65.].

Both doses will be administered orally as identical gelatine capsules.

The control arm will receive 1 mg/70 kg of psilocybin, a dose that has been shown to be inactive. Participants will be told they will receive psilocybin, but they will be blinded as to whether a low or a high dose is provided. Treating therapists, raters and researchers will also be blinded to medication group.

A single psilocybin dose will be given (either a low or a high dose) as part of a 6-hour psychedelic-assisted psychotherapy session. Additional psychotherapy sessions will be provided before (−3 days) and after (+1 day and +2 weeks) psilocybin administration.

To assess tolerability and safety profile of psilocybin in the clinical population, statistical analysis will be preformed on both continuous variables (e.g. vital signs like blood pressure and heart rate) as well as binary variables (presence vs absence of adverse reactions). Continuous variables related to adverse reactions will be compared between treatment groups (low-psilocybin vs high-psilocybin) using an independent t-test and then modelled using a mixed model repeated measures (MMRM) approach based on fixed, categorical effects of treatment dose and covariates (e.g. age, BMI, clinical site). For the binary variables, adverse events recorded at both 1 day and 2 weeks after the psilocybin session will be contrasted between treatment and controls groups using Fisher's exact test. The same approach will be used to model psilocybin efficacy: an independent t-test and a MMRM model will be constructed with continuous MADRS score as the output; Fisher's exact test will be used to compare the number of participants in the responder vs non-responder categories 1 day and 2 weeks after the psilocybin session. MADRS scores from the baseline, 1-day and 2-week timepoints will also be used to perform trajectory analysis using the R package SantaR. This method fits a cubic spline model to the time-series data and allows treatment groups to be compared based on their time evolutions.

Multivariate analysis will be carried out to compare the blood profiles of MDD participants before and after treatment with psilocybin. The concept of volatility (described in Bastiaanssen, T. F. S., et al., Volatility as a Concept to Understand the Impact of Stress on the Microbiome. Psychoneuroendocrinology, 2021. 124: p. 105047) will be used as a measure of change in the genetic, epigenetic and metabolic signatures (quantified by scRNA-seq, ChIP-seq and LC-MS) between baseline and one day post-treatment, or between baseline and 2 weeks post-treatment. Correlation analyses will be performed to determine whether volatility measures are associated with the extent of the antidepressant effect of psilocybin occurring during the same period of time (baseline-1 day or baseline-2 weeks).

A range of supervised and unsupervised multivariate techniques will be used to explore the molecular data (genetic, epigenetic and metabolic) collected from the participants at baseline. For example, unsupervised principal component analysis (PCA) will be used to explore intrinsic patterns in the data and determine the suitability of clustering approaches (e.g. k-means or hierarchical clustering). Clustering analysis will be performed to identify subgroups of participants based on their multivariate blood signatures, to determine whether the clusters reflect differences in efficacy or side effects. Next, the supervised projection to latent structures (PLS) regression will be used to relate these biological signatures to an outcome of interest, either continuous (PLS regression) or categorical (PLS discriminant analysis or PLS-DA). To identify predictive biomarkers of antidepressant response), these outcomes will be either percentage improvement in MADRS score (continuous) or response group (responders vs non-responders, categorical). To identify predictive biomarkers of adverse reactions), these outcomes will be vital signs (continuous) or AE group (with or without AEs, categorical).

MDD is a sexually-dimorphic disease, with females being twice more likely to be diagnosed than men [Eid, R. S., A. R. Gobinath, and L. A. M. Galea, Sex differences in depression: Insights from clinical and preclinical studies. Prog Neurobiol, 2019. 176: p. 86-102]. Sex differences in psilocybin response, incidence and nature of adverse effects and association with biological measures will be considered.

### Example 4

Major Depressive Disorder (MDD) is heterogeneous in nature having disease subtypes with different symptomatic (and biological) profiles. Some subsets of depressed patients present with immune activation and higher levels of inflammatory markers than non-depressed individuals.

Both the SSRI escitalopram and the psychedelic psilocybin primarily exert their antidepressant effect by enhancing serotonergic neurotransmission. However, both drugs also appear to possess anti-inflammatory properties, in line with the known anti-inflammatory action of serotonin.

The gut-brain axis is a term used to describe the bidirectional crosstalk between the gut (and the trillions of microbes residing in it; the gut microbiota) and the brain. The immune system is a major component of this network and is in constant communication with the gut microbiota.

As a component of the gut-brain axis, the gut microbiota has been established as an important contributor to health, and is implicated in a range of brain disorders, including MDD. ABCF1 appears to link inflammatory disease, the gut microbiome and depression with studies suggesting that the ABCF1 switch may be responsive to signals from the gut microbiota and is a potential therapeutic target for inflammatory diseases and psychiatric illness.

Upregulating ABCF1 function with escitalopram and psilocybin is proposed to beneficially impact microbe-immune interactions and lead to a reduction in MDD severity.

Determine Whether the Disruption of ABCF1 Function Exacerbates Inflammatory Processes and, Subsequently, Depressive-Like Behaviours, and Whether Escitalopram and Psilocybin Reverse these Effects.

ABCF1+/− mouse model: To study ABCF1 expression and function in development and disease, an ABCF1 heterozygous knock-out mouse model was created, ABCF1+/−³⁸. Adult ABCF1+/− mice are fertile and appear developmentally normal under non-stressed conditions³⁸. ABCF1 expression in embryos is required for development and no live-born ABCF1−/− progeny were ever generated. When placed under immune stress, adult ABCF1+/− mice are unable to properly regulate the switch between proinflammatory and anti-inflammatory states, and this may have profound implications on the composition of their microbiome and behaviour.

Sex and Gender Considerations: Depression is reported more frequently in women compared to men. Sex differences will be directly tested.

### Escitalopram and Psilocybin Upregulates ABCF1's Anti-Inflammatory Function.

To examine the role of ABCF1 in the anti-inflammatory action of escitalopram and psilocybin in ABCF1 knock-outs heterozygous (ABCF1+/−) and homozygous (ABCF1−/−) genetic knock-outs of ABCF1 in macrophages/microglia will be generated to determine if ABCF1 function is required for the anti-inflammatory effects of escitalopram and psilocybin. CRISPR-Cas9⁴¹⁻⁴³ will be used to knock out ABCF1 in both primary and cultured cell lines: primary splenic macrophages, brain microglia, RAW cells, THP-1 cells (a cultured macrophage cell line capable of M1 and M2 polarization⁴⁴) and BV2 microglia cell line.

RNAseq will be used to investigate gene expression changes in the knock-outs to understand (i) the effect of genetic deletion of ABCF1 and (ii) test the ability of escitalopram/psilocybin to rescue this phenotype. Escitalopram and psilocybin is expected to reduce inflammation by upregulating ABCF1 and, therefore, these drugs are expected to fail to reduce inflammation in ABCF1−/− macrophages/microglia. Showing that the anti-inflammatory properties of escitalopram/psilocybin are ablated in ABCF1−/− cells, but are rescued in ABCF1−/− reconstituted with ABCF1 would prove that ABCF1 is necessary and responsible for the anti-inflammatory phenotype induced by these drugs.

To test ABCF1+/− mice for altered microbiota profiles and altered immune profiles, 3 male and 3 female mice will be pretreated for each time point at 4 week old ABCF1+/− mice and their age- and sex-matched WT littermates for 4-8 weeks with escitalopram (50 mg/kg) or psilocybin (25 mg/kg) in the water. Immune cell profiles and microbiota composition will be tested at 4 weeks (baseline), 8 weeks (after 4 weeks of escitalopram/psilocybin pre-treatment), and 12 weeks (after 8 weeks of escitalopram/psilocybin pre-treatment). 3 male and 3 female Untreated WT and ABCF1 (+/−) mice will be included as controls for each time point.

The effect of loss of ABCF1 has on the total numbers of immune cells in vivo, and on their ability to be activated in vivo will be determined. Analysis of the immune cells within the blood will allow us to track the overall numbers and ratios (e.g., CD4/CD8 T cells) and activation status of the cells in ABCF1+/− mice versus WT mice. Peripheral blood samples will be taken from mice, and the overall cellularity of immune cells will be determined by CyTOF (mass cytometry) for high-parameter single-cell phenotyping to monitor systemic immune changes between conditions⁵¹. Flow cytometry will be used to monitor immune cells by staining with specific markers: B cells (e.g., CD19, B220, IgM, IgD, CD20, CD40, CD138 and IAb); and CD4+ T cells (e.g., CD4, CD25, CD44 and CD62L). CD4+ T cell subsets will be established by staining for canonical transcription factors and cytokines (Th1-T-bet; IFNgamma; Th2-Gata3, IL-4; Th17-RORgT, IL-17; Treg-Foxp3, IL-10). We will also assess CD8+ T cells (e.g., CD8, CD25, CD44, CD62L, PD-1 and CD127); monocytes (e.g., CD11b, F4/80); and NK cells (e.g., CD335, CD69), as well as the expression of markers of activation (e.g., CD69, IAb), memory (e.g., CD62-L, CD44, CD127) and exhaustion (e.g., PD-1, CTLA-4). From blood samples, both macrophage and microglia M1 to M2 skewing using flow cytometry will be tested. Proinflammatory and anti-inflammatory cytokine production will be studied using the commercial kit described above. Serum levels will be established by examining CRP and cytokines levels (including TNFα, IL-6, IL-10, MCP-1, IFNγ, and IL-12) using the Cytometric Bead Array Mouse Inflammation Kit. Serum IL-1β production will be analyzed using the Quantikine Mouse IL-1β/IL-1F2 ELISA kit.

Colon, liver, lung, brain, abdominal lymph nodes, and spleen tissues will also be stained for the presence of infiltrating immune cells and cytokine production, and to examine changes in tissue histology between 3 male and 3 female WT and ABCF1+/− mouse tissues. Tissue sections will be embedded in Tissue-Tek O.C.T. media (Sakura) on dry ice and immediately stored at −80° C. until sectioning. Ten microns (10 μm) thick sections will be collected on a Leica cryostat and fixed in cold acetone or acetone: methanol. Following washing in Tris-buffered saline (TBS, pH 7.4), slides will be incubated with protein block and subsequently with specific antibodies overnight (e.g., T cells: CD4, CD8, FoxP3; B cells: CD19, CD45R, B220; Granulocytes: Ly-6G; Monocytes: CD11b, Mac-1; NK cells: CD335; Cytokines: IL-6, IL-1β, TNFα, IFNγ, IFNβ, and IL-10). Appropriate horseradish peroxidase (HRP) conjugated secondary antibodies will be used for detection of the primary antibodies and developed with DAB chromogen. Slides will be counterstained with haematoxylin and eosin (H&E) to highlight tissue morphology, and dehydrated in ethanol and xylene. Giemsa staining will be used to detect eosinophils. Slides will be imaged with an Aperio ScanScope at 20×-40× magnification.

In order to study the effects of escitalopram/psilocybin and ABCF1 genotype on the microbiota, fecal pellets from ABCF1+/− mice and WT littermates⁵² will be collected. The 16S microbial community will be analysed by examining the extracted fecal DNA. All samples will be amplified by PCR in triplicate using barcoded primer pairs flanking the V3 region of the 16S gene, as previously described⁵³. Controls without template DNA will be included to ensure that no contamination occurs. Amplicons will be run on a 2% agarose gel to ensure adequate amplification. Amplicons displaying bands at ˜160 bp will purified using the illustra GFX PCR DNA Purification kit. Purified samples will be diluted 1:50 and quantified using PicoGreen (Invitrogen) in the TECAN M200 (excitation at 480 nm and emission at 520 nm). Pooled PCR amplicons will be diluted to 20 ng/ml and sequenced at the V3 hypervariable region using Hi-Seq 2000 bidirectional Illumina sequencing and Cluster Kit v4 (Macrogen Inc.). Library preparation will be done using TruSeq DNA Sample Prep v2 Kit (Illumina) with 100 ng of DNA sample and QC library by Bioanalyzer DNA 1000 Chip (Agilent).

To further study the role of ABCF in the immune response, mice (ABCF1+/− and WT littermates) will be pretreated with escitalopram or psilocybin for 4 weeks prior to challenging them with LPS. Peripheral blood cytokine profiles and immune cell diversity and changes to the gut microbiota will be assessed. We expect that pretreating the mice with escitalopram or psilocybin will result in increased ABCF1 production, allowing the ABCF1+/− mice to transition into the endotoxin-tolerant (ET) anti-inflammatory phase. We will also rechallenge mice with a non-lethal dose of LPS only (0.1-10 mg/kg) to test for changes over a longer period of time (1 week versus 2 days). Challenge with other pathogen-associated molecular patterns (PAMPSs, i.e. synthetic dsRNA analog poly riboinosinic-poly ribocytidylic acid (Poly I:C); unmethylated CpG oligodeoxynucleotide DNA⁵⁵) will be done using starting amounts that have previously shown to induce cytokine production in mice, and with titrations.

To measure cellular immune responses peripheral blood monocytic cells (PBMCs) will be obtained at time 0, and at 6 hr, 24 hr and 48 hr post-treatment with PAMPS. We will harvest PBMCs to compare lymphocyte populations as described above⁵⁶. Inflammatory cytokines profiling will be performed using ELISA kits (MBL Intl). Quantitative reverse transcriptase polymerase chain reaction (qRT-PCR) will also be used to verify cytokine production from isolated cells and tissues.

Data obtained by flow cytometry and ELISA will be expressed as mean±standard error of the mean. In multiple group comparisons, data will be analyzed by one-way ANOVA with Tukey posthoc test; P-values<0.05 considered significant. Microbiome analysis will be performed in R-studio (R-Studio, Boston, MA) using available Bioconductor packages. Sequences will be preprocessed, de-noised, and quality filtered by size using Mothur. Representative sequences will be clustered into operational taxonomic units using CrunchClust and classified against the Greengenes Database according to 97% similarity. Any taxonomic units present less than five times among all samples will removed from the analysis. Fecal microbial diversity, principal components analysis and the relative abundance of bacterial taxa will be assessed using Phyloseq.

To determine if ABCF1+/− mice exhibit increased depression-like symptoms and whether escitalopram and psilocybin can reverse depression in these mouse models a chronic social defeat stress paradigm will be used to induce depressive-like behaviour in ABCF1+/− and WT mice, which will be treated with either escitalopram/psilocybin (to boost ABCF1 expression)³⁷ or MCC950 (as a positive control). Chronic social defeat stress is a very well-established paradigm used to induce depression in rodents. In our study, chronic social defeat stress will be induced by introducing 20 male, 8-12-week-old WT or ABCF1+/− mice into the cage of a 4-month old male CD-1 aggressor mouse, where they will be subjected to a chronic social defeat stress for 10 minutes daily for 10 consecutive days. After 10 minutes, the mice will be separated by a perforated Plexiglas divider and maintained in sensory contact overnight. Every day for 10 days, the mice will be transferred to a different cage to experience physical defeat by a new resident mouse, and similarly maintained in sensory contact overnight. Control mice will be separated by a divider and rotated daily without experiencing physical defeat. Following the chronic social defeat and control protocols, animals will be singly housed during behavioural testing, which will be performed every other day. For testing female mice in this paradigm we will use 20 female WT or ABCF1 mice at 8-12 weeks and follow the protocol as described in Takahashi, A. et al. Establishment of a repeated social defeat stress model in female mice. Sci Rep 7, 12838 (2017). https://doi.org:10.1038/s41598-017-12811-8.

Parameters of depression will be assessed using the following rodent behavioural tests^{66,67}. 10 male and 10 female 8-12-wk-old WT or ABCF1+/− mice treated with different drugs will be used to test each behaviour paradigm unless specified otherwise. Vehicle-treated age and sex-matched WT or ABCF1+/− will be used as controls.

Forced swim test: Exposure to an inescapable stressor for which no active coping responses are available leads to depressive behaviour. In an open-space forced swim procedure, the mice are placed into tubs of water from which they cannot escape. Increased periods of immobility (e.g. floating) as the trial proceeds are indicative of learnt helplessness and a proxy of depressive-like behaviour.

Tail suspension test: During a 5-min trial, mice will be suspended by taping their tail to a ledge. Immobility time over the 5 min will be used as a measure of depressive behaviour.

Sucrose preference test: Mice have an innate preference for sucrose, a highly rewarding stimulus, over water. Mice will be singly-housed for a period of 16 hours. During this time, they will have access to one bottle containing water and one containing a 1% sucrose solution. After the testing period, the bottles will be weighed and sucrose preference will be calculated as a ratio of the sucrose solution to water consumed. A low sucrose preference is indicative of anhedonia-like behaviour (the inability to experience pleasure from rewarding or enjoyable activities) and will be used as a proxy of depression.

Social interaction test: This test will take place under infrared illumination in an enclosure, which will either be empty (‘no target’) or contain a novel CD1 male mouse (‘target’). Using a Raspberry Pi single-board computer and its Picam module, we will film two 150 sec sessions in which the test mouse (<12 weeks old) will be first introduced to the arena for the no-target trial, and then the target trial. MATLAB software will be used to monitor the social interaction ratio, defined as the ratio of the time spent in the interaction zone for the ‘target’ and ‘no-target’ trials. Low social interaction scores will be used as an indication of depressive-like behaviour.

The antidepressant escitalopram and the psychedelic psilocybin to dampen inflammation and reduce parameters of depression will be tested. WT and ABCF1+/− and mice will be dosed with escitalopram or psilocybin (in the water) at a concentration currently approved for use in humans⁷³ (3.2-6.4 ug/mouse for escitalopram, 1.6-3.2 ug/mouse for psilocybin), with MCC950 (40 mg/kg) 74 or vehicle control (DMSO/PBS). Depressive-like behaviour, cytokine production, immune profiles and inflammation will be analyzed as above.

Behavioural data will be expressed as mean±standard error of the mean. In multiple group comparisons, data will be analyzed by one-way ANOVA with Tukey posthoc test; P-values<0.05 considered significant.

### REFERENCES

1. Fröhlich, F. Network neuroscience. (Elsevier/Academic Press, 2016).
2. Gotlib I H, H. C. Handbook of Depression. (The Guildford Press, 2009).
3. World_Health_Organization. Depression Fact Sheet, <https://www.who.int/news-room/factsheets/detail/depression> (2020).
4. Malhi, G. S. & Mann, J. J. Lancet 392, 2299-2312, doi:10.1016/S0140-6736 (18) 31948-2 (2018).
5. Herrman, H. et al., Lancet, doi:10.1016/S0140-6736 (18) 32408-5 (2018).
6. Cepeda, M. S., et al; Journal of Clinical Psychiatry 77, 1666-1671, doi:10.4088/JCP.15m10267 (2016).
7. Zunszain, et al. in Behavioral Neurobiology of Depression and Its Treatment. Current Topics in Behavioral Neurosciences Vol. 14 Current Topics in Behavioral Neurosciences (eds P. Cowen, T. Sharp, & J. Lau) 135-151 (Springer, 2012).
8. Hasler, G. World Psychiatry: Official Journal of the World Psychiatry Association 9, 155-161 (2013).
9. Dean, J. & Keshavan, M. Asian J Psychiatr 27, 101-111, doi:10.1016/j.ajp.2017.01.025 (2017).
10. Lederberg, J. & McCray, A. T. Scientist 15, 8-8 (2001).
11. Maes, M., et al. Neuroendocrinol Lett 29, 117-124 (2008).
12. Maes, M., et al. J Affect Disorders 141, 55-62, doi:10.1016/j.jad.2012.02.023 (2012).
13. Maes, M. et al. Acta Psychiat Scand 127, 344-354, doi:10.1111/j.1600-0447.2012.01908.x (2013).
14. Clarke, G. et al. Mol Psychiatry 18, 666-673, doi:10.1038/mp.2012.77 (2013).
15. Cheung, S. G. et al. Front Psychiatry 10, 34, doi:10.3389/fpsyt.2019.00034 (2019).
16. Valles-Colomer, M. et al. Nat Microbiol 4, 623-632, doi:10.1038/s41564-018-0337-x (2019).
17. Ellul, P., et al, D. Front Neurol 9, 167, doi:10.3389/fneur.2018.00167 (2018).
18. Chen, Y. et al. Psychiatry Res 188, 224-230, doi:10.1016/j.psychres.2010.10.029 (2011).
19. Saito, S., Nakashima, A., Shima, T. & Ito, M. Am J Reprod Immunol 63, 601-610, doi:10.1111/j.1600-0897.2010.00852.x (2010).
20. Meltzer-Brody, S. Dialogues Clin Neurosci 13, 89-100 (2011).
21. Mastorakos, G. & llias, I. Ann N Y Acad Sci 997, 136-149, doi:10.1196/annals.1290.016 (2003).
22. Zhang, L., Zhang, J. & You, Z. Front Cell Neurosci 12, 306, doi:10.3389/fncel.2018.00306 (2018).
23. Rosenblat, J. D., et al Prog Neuropsychopharmacol Biol Psychiatry 53, 23-34, doi:10.1016/j.pnpbp.2014.01.013 (2014).
24. Wohleb, E. S. et al; Psychoneuroendocrinology 37, 1491-1505, doi:10.1016/j.psyneuen.2012.02.003 (2012).
25. Wohleb, E. S. et al. J Neurosci 31, 6277-6288, doi:10.1523/JNEUROSCI.0450-11.2011 (2011).
26 Czeh, M., Gressens, P. & Kaindl, A. M. Dev Neurosci 33, 199-209, doi:10.1159/000328989 (2011).
27 Venneti, S., Lopresti, B. J. & Wiley, C. A. Glia 61, 10-23, doi:10.1002/glia.22357 (2013).
28 Yirmiya, R. et al. Adv Exp Med Biol 461, 283-316, doi:10.1007/978-0-585-37970-8_16 (1999).
29 Rock, R. B. et al. Clin Microbiol Rev 17, 942-964, (2004).
30 Vollmer-Conna, U. et al. Psychol Med 34, 1289-1297, doi:10.1017/s0033291704001953 (2004).
31 Dowlati, Y. et al. Biol Psychiatry 67, 446-457, doi:10.1016/j.biopsych.2009.09.033 (2010).
32 Haapakoski, R., et al. Brain Behav Immun 49, 206-215, doi:10.1016/j.bbi.2015.06.001 (2015).
33. Reichenberg, A. et al. Arch Gen Psychiatry 58, 445-452, doi:10.1001/archpsyc.58.5.445 (2001).
34. Grigoleit, J. S. et al. PLOS One 6, e28330, doi:10.1371/journal.pone.0028330 (2011).
35. Harrison, N. A. et al., Biol Psychiatry 66, 407-414, (2009).
36. Yirmiya, R. Brain Res 711, 163-174, (1996).
37. Yirmiya, R. et al. Neuropsychopharmacol 24, 531-544, doi:10.1016/S0893-133X (00) 00226-8 (2001).
38. Henry, C. J. et al. J Neuroinflammation 5, 15, doi:10.1186/1742-2094-5-15 (2008).
39. O'Connor, J. C. et al. Mol Psychiatry 14, 511-522,doi:10.1038/sj.mp.4002148 (2009).
40. O'Connor, J. C. et al. J Neurosci 29, 4200-4209, doi:10.1523/JNEUROSCI.5032-08.2009 (2009).
41. Dobos, N. et al., J Alzheimers Dis 28, 905-915, doi:10.3233/JAD-2011-111097 (2012).
42. Fenn, A. M. et al. Biol Psychiatry 76, 575-584, doi:10.1016/j.biopsych.2013.10.014 (2014).
43. Corona, A. W. et al. Brain Behav Immun 31, 134-142, doi:10.1016/j.bbi.2012.08.008 (2013).
44. Li, Z. Q., et al., Biochem Biophys Res Commun 501, 933-940, doi:10.1016/j.bbrc.2018.05.085 (2018).
45. Garcia-Barrio, M., Dong, J., Ufano, S. & Hinnebusch, A. G. EMBO J 19, 1887-1899, doi:10.1093/emboj/19.8.1887 (2000).
46. Marton, M. J., et al. Mol Cell Biol 17, 4474-4489 (1997).
47. Paytubi, S., et al. Biochem J 409, 223-231, doi: BJ20070811 [pii] 10.1042/BJ20070811 (2008).
48. Campbell, S. G., et al. J Cell Biol 170, 925-934, doi:10.1083/jcb.200503162 (2005).
49. Pestova, T. V. & Hellen, C. U. Nat Struct Mol Biol 13, 98-99, doi:10.1038/nsmb0206-98 (2006).
50. Richard, M., Drouin, R. & Beaulieu, A. D. Genomics 53, 137-145, doi: S0888-7543 (98) 95480-5 [pii] 10.1006/geno. 1998.5480 (1998).
51. Ota, M. et al. Immunogenetics 59, 45-52, doi:10.1007/s00251-006-0178-2 (2007).
52. Zhu, H. et al. PLOS One 11, e0167212, doi:10.1371/journal.pone.0167212 (2016).
53. Arora, H. et al. Immunity 50, 418-431 e416, doi:10.1016/j.immuni.2019.01.014 (2019).
54. Brunton, L., et al The Pharmacological Basis of Therapeutics. 12 edn, (McGraw Hill Professional, 2010).
55. Horowitz, M. A. et al. Int J Neuropsychopharmacol 18, doi:10.1093/ijnp/pyu076 (2014).
56. Abdel-Salam, O. M., et al Pharmacol Res 48, 157-165 (2003).
57. Roumestan, C. et al. Respir Res 8, 35, doi:10.1186/1465-9921-8-35 (2007).
58. Tynan, R. J. et al. Brain Behav Immun 26, 469-479, doi:10.1016/j.bbi.2011.12.011 (2012).
59. Al-Amin, M. M., et al. Inflammopharmacology 21, 421-425, doi:10.1007/s10787-013-0181-9 (2013).
60. Cattaneo, A. et al. Neuropsychopharmacology 38, 377-385, doi:10.1038/npp.2012.191 (2013).
61. Kenis, G. & Maes, M. Int J Neuropsychopharmacol 5, 401-412, doi:10.1017/S1461145702003164 (2002).
62. Kubera, M. et al. J Clin Psychopharmacol 21, 199-206 (2001).
63. Powell, T. R. et al. Eur Neuropsychopharmacol 23, 1105-1114, doi:10.1016/j.euroneuro.2012.09.009 (2013).
64. Uher, R. et al., Am J Psychiatry 167, 555-564, doi:10.1176/appi.ajp.2009.09070932 (2010).
65. Malki, K. et al., Pharmacogenet Genomics 22, 765-776, doi:10.1097/FPC.0b013e328356fa90 (2012).
66. Powell, T. R. et al. J Psychopharmacol 27, 609-615, doi:10.1177/0269881113490329 (2013).
67. Wilcox, S. M. et al. Plos One 12, e0175918, doi:10.1371/journal.pone.0175918 (2017).
68. Kimura-Ohba, S. et al. J Cereb Blood Flow Metab 36, 1731-1743, doi:10.1177/0271678X16630556 (2016).
69. Albert, P. R. J Psychiatry Neurosci 40, 219-221 (2015).
70. Yurist-Doutsch, S. et al., Plos One 11, e0159676, doi:10.1371/journal.pone.0159676 (2016).
71. Bartram, A. K et al. Appl Environ Microbiol 77, 3846-3852, doi:10.1128/AEM.02772-10 (2011).
72. Schloss, P. D. et al. Appl Environ Microbiol 75, 7537-7541, doi:10.1128/AEM.01541-09 (2009).
73. Hartmann, M. et al. ISME J 6, 2199-2218, doi:10.1038/ismej.2012.84 (2012).
74. DeSantis, T. Z. et al. Appl Environ Microbiol 72, 5069-5072, doi:10.1128/AEM.03006-05 (2006).
75. McMurdie, P. J. & Holmes, S. Plos One 8, e61217, doi:10.1371/journal.pone.0061217 (2013).
76. Christoffel, D. J. et al. J Neurosci 31, 314-321, doi:10.1523/JNEUROSCI.4763-10.2011 (2011).
77. Coll, R. C. et al. Nat Med 21, 248-255, doi:10.1038/nm.3806 (2015).
78. Golden, S. A., et al Nat Protoc 6, 1183-1191, doi:10.1038/nprot.2011.361 (2011).
79 McGirr, A., et al. Brain 140, 2210-2225, doi:10.1093/brain/awx142 (2017).
80 Brady, A. M. & Floresco, S. B. Vis Exp, e52387, doi:10.3791/52387 (2015).
81 Weiss, J. M., et al J Comp Physiol Psychol 72, 153-160 (1970).
82 Wagner, H. R., 2nd, Hall, T. L. & Cote, I. L. J Gen Psychol 96, 313-318, doi:10.1080/00221309.1977.9920828 (1977).
83 Porsolt, R. D., et al., Arch Int Pharmacodyn Ther 229, 327-336 (1977).
84 Steru, L., et al., Psychopharmacology (Berl) 85, 367-370 (1985).
85 Liu, M. Y. et al. Nat Protoc 13, 1686-1698, doi:10.1038/s41596-018-0011-z (2018).
86 Sunyer, B., et al. Barnes maze, a useful task to assess spatial reference memory in the mice (2007).

1. A method of upregulating ABCF1 expression in a patient in need thereof, comprising administering one or more agonists of ABCF1.

2. The method of claim 1, wherein said one or more agonists of ABCF1 comprises a cannabinoid.

3. The method of claim 2, wherein said cannabinoid is selected from the group consisting of Cannabigerol, Cannabichromene, Cannabidiol, Tetrahydrocannabinol, Cannabinol, Cannabielsoin, iso-Tetrahydrocannabinol, Cannabicyclol and derivatives thereof.

4. The method of claim 2, wherein said cannabinoid is cannabigerol.

5. The method of claim 1, wherein said one or more agonists ABCF1 is a natural product.

6. The method of claim 5, wherein said natural product is from Ascomycetes fungus.

7. The method of claim 4, wherein the natural product is from Cordyceps sinensis.

8. The method of claim 5, wherein said natural product is from a mushroom.

9. The method of claim 5, wherein the natural product is fresh, dried or an extract.

10. The method of claim 1, wherein said agonist of ABCF1 is a Psilocybin analog.

11. The method of claim 10, wherein said Psilocybin analog is selected from the group consisting of 4-Acetoxy-N, N-dimthyltryptamine; O-Acetyl Psilocin Fumerate; 4-acetoxyindole; 4-Acetoxy-N-isopropyl-N-methyltryptamine; 4-Acetoxy-N-ethyl-N-methyltryptamine; Acetoxy-N,N-diethyltryptamine; 4-AcO-DET Fumarate and. 4-Acetoxy-N-ethyl-N-methyltryptamine Fumarate.

12. The method of claim 1, wherein upregulating ABCF1 expression inhibits neuroinflammation.

13. The method of claim 12, wherein inhibiting neuroinflammation treats or alleviates one or more symptoms of depression in said patient.

14. The method of claim 1, wherein upregulating ABCF1 expression treats Major Depressive Disorder.

15. The method of claim 13, wherein inhibiting neuroinflammation treats or alleviates one or more symptoms of Major Depressive Disorder (MDD), postpartum depression, schizophrenia, anxiety, bipolar disorder, obsessive-compulsive disorder (OCD), posttraumatic stress disorder (PTSD), and autism spectrum disorder.

16. The method of claim 12, wherein inhibiting neuroinflammation treats an autoimmune disease and comorbid neuropsychiatric disorders.

17. The method of claim 1, wherein said one or more agonists are administered as one or more micro doses.

18. The method of claim 1, wherein said one or more agonists are administered in combination with one or more other therapeutics.

19. The method of claim 15, wherein said one or more symptoms are selected from the group consisting of trouble concentrating, remembering details, and making decisions; fatigue; feelings of guilt, worthlessness, and helplessness; pessimism and hopelessness; insomnia, early-morning wakefulness, or sleeping too much; irritability; restlessness; loss of interest in things once pleasurable, including sex; overeating, or appetite loss; aches, pains, headaches, or cramps that won't go away; digestive problems that don't get better, even with treatment; persistent sad, anxious, or “empty” feelings; and suicidal thoughts or attempts.
